(19) United States
(12) Patent Application Publication
(10) Pub. No.: US 2025/0333530 A1
(43) Pub. Date: Oct. 30, 2025

(54) COMBINATION TREATMENT OF GLOFITAMAB AND CHEMOTHERAPY

(71) Applicants: Hoffmann-La Roche Inc., Little Falls, NJ (US); Hoffmann-La Roche Limited, Mississauga (CA); Genentech, Inc., South San Francisco, CA (US)

(72) Inventors: Mark Francis DIXON, Welwyn Garden City (GB); Stephen James Simko, III, South San Francisco, CA (US); Victor Manuel Orellana-Noia, South San Francisco, CA (US); Martine Joanna Kallemeijn, Basel (CH); Linda Maria Lundberg, Basel (CH); Yuying Xie, Mississauga (CA); Marina Bacac, Zuerich (CH); Johannes Sam, Schlieren (CH); James Christopher Relf, Welwyn Garden City (GB)

(21) Appl. No.: 19/175,703

(22) Filed: Apr. 10, 2025

Related U.S. Application Data
(60) Provisional application No. 63/633,653, filed on Apr. 12, 2024.
(60) Provisional application No. 63/654,678, filed on May 31, 2024.
(60) Provisional application No. 63/689,263, filed on Aug. 30, 2024.

(51) Int. Cl. C07K 16/28; A61K 31/555; A61K 31/573; A61K 31/7068; A61K 39/00; A61P 35/00

(52) U.S. Cl. CPC C07K 16/2887; A61K 31/555; A61K 31/573; A61K 31/7068; A61P 35/00; C07K 16/2809; A61K 2039/505; A61K 2039/545

(57) ABSTRACT

The present invention relates to methods of treating B-cell proliferative disorders, e.g., primary refractory or relapsed diffuse large B-cell lymphoma (DLBCL), by administering glofitamab in combination with gemcitabine and oxaliplatin. Further the invention related to an optimized corticosteroid prophylaxis for glofitamab resulting in lower incidence of cytokine release syndrome (CRS).

### SEQUENCE LISTING

The instant application contains a Sequence Listing which has been submitted electronically in XML format and is hereby incorporated by reference in its entirety. Said XML copy, created on Mar. 20, 2025, is named 51177-055004_Sequence_Listing_3_20_25.xml and is 55,367 bytes in size.

### FIELD OF THE INVENTION

The present invention relates to methods of treating B-cell proliferative disorders, e.g., refractory or relapsed diffuse large B-cell lymphoma (DLBCL), by administering glofitamab and in combination with gemcitabine plus oxaliplatin (GemOx).

### BACKGROUND OF THE INVENTION

Non-Hodgkin lymphoma (NHL) is the most common hematologic malignancy in the world and the thirteenth most common cancer overall (Bray et al. CA Cancer J Clin. 68 (6): 394-424, 2018). It is estimated that 509,590 new cases of NHL were diagnosed worldwide in 2018 (2.8% of the total new cancer cases) and 248,724 people died of the disease (2.6% of total cancer-related deaths). The age-standardized risks of newly diagnosed NHL across Northern, Southern, Eastern, and Western Europe ranged from 280.1 to 363.5 per 100,000 person-years for males and from 216.5 to 292.1 per 100,000 person-years for females. The age-standardized risks of mortality from NHL across the same European regions were 118.4-171.0 and 76.2-92.0 per 100,000 person-years, respectively. In the United States, it is estimated that 74,680 people were diagnosed with NHL in 2018 (incidence, 19.4 per 100,000) and 19,910 patients died from the disease (National Cancer Institute [NCI] 2018).

NHL comprises a heterogeneous group of lymphoproliferative disorders but most commonly presents as a defect in B lymphocytes. Diffuse large B-cell lymphoma (DLBCL) is the most common subtype of NHL of B-cell origin (30%-40% of the total NHL cases) (Al-Hamadani et al. Am J Hematol. 90 (9): 790-795, 2015).

Originating from mature B cells, DLBCL is an aggressive NHL with a median survival of <1 year in untreated patients (Rovira et al. Ann Hematol. 94 (5): 803-812, 2015). Despite its aggressive disease course, approximately 50%-70% of patients may be cured with the current standard-of-care treatment that consists of rituximab in combination with cyclophosphamide, doxorubicin, vincristine, and prednisone (R-CHOP) chemotherapy (Flowers et al. CA Cancer J Clin. 60 (6): 393-408, 2010). Nevertheless, R-CHOP is found to be inadequate in 30%-50% of patients because of either primary refractoriness or relapse after achieving a complete response (CR). Elderly patients remain a particularly difficult subset to treat given their reduced tolerance to cytotoxic chemotherapy. Improved therapies having improved efficacy and/or improved safety profiles for treating DLBCL are needed.

### SUMMARY OF THE INVENTION

The present invention features methods of treating patients with relapsed or refractory diffuse large B-cell lymphoma (DLBCL). These patients may have relapsed after or were refractory after one prior line of therapy and are not candidates for transplant; or these patients may have relapsed after or were refractory to two or more prior lines of therapy. Patients having relapsed or refractory DLBCL may exhibit improved response rates or more durable responses after treatment with the presently claimed methods, e.g., including a combination of glofitamab with gemcitabine and oxaliplatin (GemOx), e.g., in comparison with a control treatment, e.g., comprising rituximab, gemcitabine, and oxaliplatin (R-GemOx). Patients treated with the presently claimed methods may also exhibit improved response and/or receptibility to autologous stem cell transplant (ASCT) or chimeric antigen receptor T cell therapy, e.g., in comparison to post-control treatment, e.g., comprising R-GemOx.

In one aspect, the invention features a method of treating relapsed or refractory DLBCL in a human patient in need thereof, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
in the absence of glofitamab.

In one embodiment, the PFS or the reference PFS is measured starting from the time from randomization to the time of a first occurrence of disease progression or death from any cause.

In one embodiment, the PFS or the reference PFS is the median PFS of the plurality of human patients receiving the corresponding treatment.

In one embodiment, the improvement in PFS is statistically significant.

In one embodiment, the improvement of the median PFS is an increase in the PFS compared to the reference PFS of between 1 and 18 months.

In one embodiment, the improvement of the median PFS is an increase in the PFS compared to the reference PFS of about 9 months. In one embodiment, the improvement of the median PFS is an increase in the PFS compared to the reference PFS of about 10 months.

In one embodiment, administering such treatment to a plurality of human patients results in a statistically significant improvement in the PFS as compared to the control treatment with a hazard ratio of about 0.42. In one embodiment, administering such treatment to a plurality of human patients results in a statistically significant improvement in the PFS as compared to the control treatment with a hazard ratio of about 0.40.

In one embodiment, administering such treatment to a plurality of human patients results in a statistically significant improvement in the PFS as compared to the control treatment with a hazard ratio of about 0.42 (95% confidence interval: 0.29, 0.61). In one embodiment, administering such treatment to a plurality of human patients results in a statistically significant improvement in the PFS as compared to the control treatment with a hazard ratio of about 0.40 (95% confidence interval: 0.28, 0.57). In one embodiment, administering such treatment to a plurality of human patients results in a statistically significant improvement in the PFS as compared to the control treatment with a hazard ratio of about 0.41 (95% confidence interval: 0.29, 0.58).

In one embodiment, the hazard ratio is a stratified hazard ratio.

In one embodiment, administering such treatment to a plurality of human patients results in an increase in the rate of PFS compared to the rate of reference PFS at 6 months of between 5% and 45%.

In one embodiment, administering such treatment to a plurality of human patients results in an increase in the rate of PFS compared to the rate of reference PFS at 6 months of about 25%.

In one embodiment, administering such treatment to a plurality of human patients results in an increase in the rate of PFS compared to the rate of reference PFS at 12 months of between 5% and 45%.

In one embodiment, administering such treatment to a plurality of human patients results in an increase in the rate of PFS compared to the rate of reference PFS at 12 months of about 25%.

In one embodiment, administering such treatment to a plurality of human patients results in an improvement of the complete response rate (CR rate), objective response rate (ORR), duration of objective response, and/or duration of CR (DOCR) as compared to the control treatment.

In one embodiment, the CR rate is the proportion of patients whose best overall response is a CR on PET/computed tomography (CT).

In one embodiment, the improvement of the CR rate is an increase of between 15% and 50%.

In one embodiment, the improvement of the CR rate is an increase of about 30%.

In one embodiment, the ORR is the proportion of patients whose best overall response is a partial response (PR) or a CR.

In one embodiment, the duration of objective response is measured as the time from the first occurrence of a documented objective response (CR or PR) to disease progression, or death from any cause, whichever occurs first.

In one embodiment, the DOCR is measured as the time from the first occurrence of a documented CR to disease progression, or death from any cause, whichever occurs first.

In one aspect, a method for treating relapsed or refractory diffuse large B-cell lymphoma (DLBCL) in a human patient in need thereof is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab.

In one embodiment, the OS or the reference OS is measured starting from the time from randomization to death from any cause.

In one embodiment, the OS or the reference OS is the median OS of the plurality of human patients receiving the corresponding treatment.

In one embodiment, the improvement in OS is statistically significant.

In one embodiment, the improvement of the median OS is an increase in the OS compared to the reference OS of between 1 and 30 months.

In one embodiment, the improvement of the median OS is an increase in the OS compared to the reference OS of about 13 months.

In one embodiment, administering such treatment to a plurality of human patients results in a statistically significant improvement in the OS as compared to the control treatment with a hazard ratio of about 0.62.

In one embodiment, administering such treatment to a plurality of human patients results in a statistically significant improvement in the OS as compared to the control treatment with a hazard ratio of about 0.62 (95% confidence interval: 0.43, 0.88). In some embodiments, administering such treatment to a plurality of human patients results in a statistically significant improvement in the OS as compared to the control treatment with a hazard ratio of about 0.60 (95% confidence interval: 0.42, 0.85).

In one embodiment, the hazard ratio is a stratified hazard ratio.

In one embodiment, administering such treatment to a plurality of human patients results in an increase in the rate of OS compared to the rate of reference OS at 12 months of between 5% and 30%.

In one embodiment, administering such treatment to a plurality of human patients results in an increase in the rate of OS compared to the rate of reference OS at 12 months of about 10%.

In one embodiment, administering such treatment to a plurality of human patients results in an increase in the rate of OS compared to the rate of reference OS at 18 months of between 5% and 35%.

In one embodiment, administering such treatment to a plurality of human patients results in an increase in the rate of OS compared to the rate of reference OS at 18 months of about 20%.

In one embodiment, administering such treatment to a plurality of human patients results in an increase in the rate of OS compared to the rate of reference OS at 24 months of between 5% and 40%.

In one embodiment, administering such treatment to a plurality of human patients results in an increase in the rate of OS compared to the rate of reference OS at 24 months of about 20%.

In one embodiment, the stratified hazard ratio is stratified by: (a) the number of previous lines of systemic therapy for DLBCL (1 vs. ≥2)); and/or (b) the outcome of last systemic therapy (relapsed vs. refractory).

In one aspect, the invention features a method for treating relapsed or refractory diffuse large B-cell lymphoma (DLBCL) in a human patient in need thereof, comprising administering to the human patient an effective amount of: (a) glofitamab, (b) gemcitabine, and (c) oxaliplatin, wherein administering such treatment to a plurality of human patients results in a median duration of complete remission in the plurality of human patients of at least 27 months.

In one aspect, the invention features a method for treating relapsed or refractory diffuse large B-cell lymphoma (DLBCL) in a human patient in need thereof, comprising administering to the human patient an effective amount of: (a) glofitamab, (b) gemcitabine, and (c) oxaliplatin, wherein administering such treatment to the patient results in a complete remission in the patient, and wherein a plurality of human patients having received such treatment and exhibiting complete remission after end of treatment exhibits a rate of overall survival at 12 months of about 89%.

In one aspect, the invention features a method for treating relapsed or refractory diffuse large B-cell lymphoma (DLBCL) in a human patient in need thereof, comprising administering to the human patient an effective amount of: (a) glofitamab, (b) gemcitabine, and (c) oxaliplatin, wherein administering such treatment to the patient results in a complete remission in the patient, and wherein a plurality of human patients having received such treatment and exhibiting complete remission after end of treatment exhibits a rate of progression-free survival at 12 months of about 82%.

In one embodiment, the method comprises a first and a second dosing cycle, wherein:
the first dosing cycle comprises a first dose (C1D1) of about 2.5 mg of the glofitamab and a second dose (C1D2) of about 10 mg of the glofitamab, and
the second dosing cycle comprises a single dose (C2D1) of about 30 mg of the glofitamab.

In one embodiment, the C1D1 and the C1D2 of the glofitamab are administered to the patient on Days 8 and 15, respectively, of the first dosing cycle.

In one embodiment, the C2D1 of the glofitamab is administered to the patient on Day 1 of the second dosing cycle.

In one embodiment, the first dosing cycle comprises a dose of about 1000 mg of the obinutuzumab. In one embodiment, obinutuzumab is administered as a single dose of 1000 mg.

In one embodiment, the obinutuzumab is administered about 7 days before the first glofitamab dose.

In one embodiment, the obinutuzumab is administered on Day 1 of the first dosing cycle.

In one embodiment, the first and the second dosing cycles each comprises a dose of about 1000 mg/m² of the gemcitabine and a dose of about 100 mg/m² of the oxaliplatin.

In one embodiment, the gemcitabine and the oxaliplatin are administered on Day 2 of the first dosing cycle.

In one embodiment, the gemcitabine and the oxaliplatin are administered on Day 1 or 2 of the second dosing cycle.

In one embodiment, the gemcitabine is administered before the oxaliplatin on the same day.

In one embodiment, the first and second dosing cycles are each 21-day dosing cycles.

In one embodiment the method comprises twelve dosing cycles.

In one embodiment, the dosing cycles are each 21-day dosing cycles.

In one embodiment the method comprises:
dosing cycle 1, wherein the patient is administered an effective dose of the obinutuzumab, the glofitamab, the gemcitabine, and the oxaliplatin,
dosing cycles 2 to 8, wherein the patient is administered an effective dose of the glofitamab, the gemcitabine, and the oxaliplatin; and
dosing cycles 9 to 12, wherein the patient is administered an effective dose of the glofitamab

In one embodiment, the obinutuzumab is administered at a dose of about 1000 mg on Day 1 of dosing cycle 1; the glofitamab is administered at a dose of about 0.5 mg on Day 8 and about 10 mg on Day 15 of dosing cycle 1; and at a dose of about 30 mg on Day 1 of dosing cycles 2 to 12; and the gemcitabine is administered at a dose of about 1000 mg/m² and the oxaliplatin is administered at a dose of about 100 mg/m² on Day 2 of dosing cycle 1 and on Day 1 or 2 of dosing cycles 2 to 8.

In one embodiment, the gemcitabine is administered before the oxaliplatin on the same day.

In one aspect, glofitamab is provided for use in a method of treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
in the absence of glofitamab

In one aspect, the invention relates to glofitamab for use in a method of treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
in the absence of glofitamab.

In one aspect, the invention relates to use of glofitamab in the manufacture of a medicament for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
in the absence of glofitamab.

In one aspect, the invention relates to use of glofitamab in the manufacture of a medicament for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
in the absence of glofitamab.

In one aspect, the invention features a method for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein the patient has relapsed after or is refractory to one prior line of therapy and is not a candidate for HSCT, and wherein the patient:
(a) has a left ventricular ejection fraction ≤40%,
(b) has creatinine clearance or glomerular filtration rate ≤45 mL/min,
(c) has Eastern Cooperative Oncology Group (ECOG) Performance Status of ≥2,
(d) is aged 70 years or more,
(e) refused high-dose chemotherapy and/or transplant, and/or
(f) had insufficient response to pre-transplant chemotherapy to be able to proceed to transplant.

In one embodiment, the human patient has a relapsed or refractory DLBCL not otherwise specified (DLBCL NOS). In one embodiment, the human patient is not a candidate for hematopoietic stem cell transplantation (HSCT) (e.g., ineligible for HSCT). In one embodiment, the human patient has a relapsed or refractory DLBCL NOS and is not a candidate for HSCT (e.g., ineligible for HSCT).

In one aspect, the invention features a method for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein the DLBCL is a DLBCL not otherwise specified (DLBCL NOS), and wherein the patient is not a candidate for hematopoietic stem cell transplantation (HSCT).

In one aspect, the invention features a method for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein the DLBCL is a DLBCL not otherwise specified (DLBCL NOS), and wherein the patient is not a candidate for hematopoietic stem cell transplantation (HSCT).

In one embodiment, the patient receives corticosteroid prophylaxis prior to and after administration of glofitamab.

In one embodiment, the corticosteroid prophylaxis comprises prednisolone and methylprednisolone, and/or dexamethasone.

In one embodiment, the corticosteroid prophylaxis comprises dexamethasone. In one embodiment, the corticosteroid prophylaxis comprises 20 mg dexamethasone. In one embodiment, the corticosteroid prophylaxis is administered one day prior to administration of glofitamab.

In one embodiment, the corticosteroid prophylaxis is administered about 24 hours prior to administration of glofitamab. In one embodiment, the corticosteroid prophylaxis is administered on the same day as administration of glofitamab. In one embodiment, the corticosteroid prophylaxis is administered about 30-90 or about 60 minutes prior to the glofitamab administration.

In one embodiment, the corticosteroid prophylaxis is administered one day after administration of glofitamab.

In one embodiment, the corticosteroid prophylaxis is administered about 24 hours after administration of glofitamab.

In one embodiment, the corticosteroid prophylaxis is administered before the first dose (C1D1) of glofitamab.

In one embodiment, the corticosteroid prophylaxis is administered before the second dose (C1D2) of glofitamab.

In one embodiment, the corticosteroid prophylaxis is administered one day prior to the glofitamab administration, prior to the glofitamab administration on the same day, and one day after the glofitamab administration.

In one embodiment, the corticosteroid prophylaxis is administered about 24 hours prior to the glofitamab administration, about 30-90 or about 60 minutes prior to the glofitamab administration, and about 24 hours after the glofitamab administration.

In one embodiment, the corticosteroid prophylaxis is dexamethasone.

In one embodiment, dexamethasone is administered at a dose of 20 mg.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the corticosteroid prophylaxis is administered before the third dose (C2D1) of glofitamab.

In one embodiment, the corticosteroid prophylaxis is administered before any subsequent dose of glofitamab if the patient has experienced CRS with any previous 30 mg dose of glofitamab.

In one embodiment, the corticosteroid prophylaxis comprises 20 mg dexamethasone.

In one embodiment, the incidence of CRS in a plurality of patients is reduced compared to treatment wherein corticosteroid prophylaxis consists of one dose of a corticosteroid on the same day as glofitamab administration.

In one embodiment, the patient does not have a Grade 3 CRS event.

In one embodiment, the patient does not need to be hospitalized after treatment with glofitamab.

In one aspect, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
(1) the first dosing cycle comprises a first dose (C1D1) of 2.5 mg of glofitamab and a second dose (C1D2) of 10 mg of glofitamab; and
(2) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one aspect, the invention features a method of reducing the likelihood of a CRS event in a CD20-positive B cell proliferative disorder patient treated with glofitamab, comprising administering to the patient glofitamab and dexamethasone in a dosing regimen comprising at least a first dosing cycle and a second dosing cycle, wherein:
(1) the first dosing cycle comprises a first dose (C1D1) of 2.5 mg of glofitamab and a second dose (C1D2) of 10 mg of glofitamab; and
(2) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, dexamethasone is administered for the third dose (C2D1) of glofitamab if the patient has experienced CRS with first and or second dose of glofitamab.

In one embodiment, dexamethasone is administered before any subsequent dose of glofitamab if the patient has experienced CRS with any previous 30 mg dose of glofitamab.

In one embodiment, dexamethasone is administered for the first and second dose (C2D1) of glofitamab and not for any subsequent dose.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one embodiment, the method further comprises administering gemcitabine and oxaliplatin.

In one aspect, glofitamab is provided for use in a method of treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
in the absence of glofitamab,
wherein the patient receives corticosteroid prophylaxis prior to and after administration of glofitamab.

In one aspect, the invention relates to glofitamab for use in a method of treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
in the absence of glofitamab, wherein the patient receives corticosteroid prophylaxis prior to and after administration of glofitamab.

In one aspect, the invention relates to dexamethasone for use in a method of reducing the incidence of CRS events in a CD20-positive B cell proliferative disorder patient population treated with glofitamab, wherein the patients in the patient population are to be administered glofitamab and dexamethasone in a dosing regimen comprising at least a first dosing cycle and a second dosing cycle, wherein:
(1) the first dosing cycle comprises a first dose (C1D1) of 2.5 mg of glofitamab and a second dose (C1D2) of 10 mg of glofitamab; and
(2) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one aspect, the invention relates to dexamethasone for use in a method of reducing the likelihood of a CRS event in a CD20-positive B cell proliferative disorder patient treated with glofitamab, wherein patient is to be administered glofitamab and dexamethasone in a dosing regimen comprising at least a first dosing cycle and a second dosing cycle, wherein:
(1) the first dosing cycle comprises a first dose (C1D1) of 2.5 mg of glofitamab and a second dose (C1D2) of 10 mg of glofitamab; and
(2) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

Each and every embodiment can be combined unless the context clearly suggests otherwise. Each and every embodiment can be applied to each and every aspect of the invention unless the context clearly suggests otherwise.

Specific embodiments of the present invention will become evident from the following more detailed description of certain preferred embodiments and the claims.

### BRIEF DESCRIPTION OF THE DRAWINGS

FIG. 1 shows the regimen in the Study Treatment Group: Glofitamab in Combination with Gemcitabine plus Oxaliplatin (Glofit-GemOx Arm)

FIG. 2 shows the regimen in the Control Group: Rituximab in Combination with Gemcitabine plus Oxaliplatin (R-GemOx Arm) FIG. 3 is a Kaplan-Meier plot illustrating the overall survival (OS) of intent-to-treat patients administered rituximab, gemcitabine, and oxaliplatin (R-GemOx; solid line) or glofitamab, gemcitabine, and oxaliplatin (Glofit-GemOx; dashed line). Patients administered Glofit-GemOx exhibited higher OS (hazard ratio=0.59; p-value=0.0107).

FIG. 4 is a Kaplan-Meier plot illustrating the progression-free survival of intent-to-treat patients administered rituximab, gemcitabine, and oxaliplatin (R-GemOx; solid line) or glofitamab, gemcitabine, and oxaliplatin (Glofit-GemOx; dashed line). Patients administered Glofit-GemOx exhibited higher PFS (hazard ratio=0.34; p-value <0.00001).

FIG. 5 is a Kaplan-Meier plot illustrating the overall survival (OS) of R/R DLBCL patients administered rituximab, gemcitabine, and oxaliplatin (R-GemOx; solid line) or glofitamab, gemcitabine, and oxaliplatin (Glofit-GemOx; dashed line) at follow-up analysis with cut-off date of Feb. 16, 2024. Patients administered Glofit-GemOx exhibited higher OS (hazard ratio=0.62; 95% CI: 0.43-0.88).

FIG. 6 illustrates the in vivo study design to evaluate the effect of dexamethasone scheduling on CRS and efficacy of Glofitamab.

FIG. 7 shows tumor growth inhibition in mice treated with glofitamab and dexamethasone.

FIGS. 8A and 8B shows the cytokine measurement in mice after the first and second glofitamab injection, at 4 (FIG. 8A) and 28 hours (FIG. 8B) after treatment. A: Vehicle, B: glofitamab; C: glofitamab+dexamethasone (1 h prior); D: glofitamab+dexamethasone (24 h & 1 h prior+24 h after) 4 mg/kg; E: glofitamab+dexamethasone (24 h & 1 h prior) 2 mg/kg; F: glofitamab+dexamethasone (24 h & 1 h prior+24 h after) 2/4 mg/kg; G: glofitamab+dexamethasone (24 h & 1 h prior) 6/2 mg/kg.

FIG. 9 illustrates the in vivo study design to evaluate the effect of dexamethasone scheduling on CRS and efficacy of Glofitamab in combination with Gemcitabine and Oxaliplatin.

FIG. 10A to FIG. 10C illustrates the tumor growth kinetics as the mean tumor volume (FIG. 10A), individual tumor growth trajectories for each mouse within the various treatment groups (FIG. 10B), and the tumor volumes from groups D-H at study termination for statistical analysis (FIG. 10C) of the preclinical study evaluating the effect of dexamethasone scheduling on CRS and efficacy of Glofitamab in combination with Gemcitabine and Oxaliplatin. CD20-TCB=Glofitamab, Stats: One-way Anova. *: p<0.05; **: p<0.01; ***: p<0.001; ****: p<0.0001.

FIG. 11A to FIG. 11C show the cytokine measurement (FIG. 11A: IL6 and IL2; FIG. 11B: MCP-1 and IFN-γ; FIG. 11C: IL-8 and IL-10) in serum 6 hrs after last Dexamethasone administration at first cycle (day 10) of the preclinical study evaluating the effect of dexamethasone scheduling on CRS and efficacy of Glofitamab in combination with Gemcitabine and Oxaliplatin. CD20-TCB=Glofitamab, Stats: One-way Anova. *: p<0.05; **: p<0.01; ***: p<0.001; ****: p<0.0001.

FIG. 12 illustrates the study schema of study GO45434. Dexamethasone on days 7, 9, 14 and 16 administered orally. * Dexamethasone administered 60 min by intravenous infusion prior to glofitamab administration; ** Dexamethasone optional based on investigator's assessment for participants that have tolerated at least one 30 mg dose of glofitamab without experiencing CRS. Note: For Cycles 1-8, gemcitabine should be administered before oxaliplatin. For Cycles 2-8, glofitamab should be given before gemcitabine and oxaliplatin.

FIG. 13 is a Kaplan-Meier plot illustrating the overall survival (OS) of patients administered rituximab, gemcitabine, and oxaliplatin (R-GemOx; solid line) or glofitamab, gemcitabine, and oxaliplatin (Glofit-GemOx; dashed line) in the updated analysis for the STARGLO trial. Patients administered Glofit-GemOx exhibited higher OS (hazard ratio=0.62).

FIG. 14 is a Kaplan-Meier plot illustrating the progression-free survival (PFS) of patients administered rituximab, gemcitabine, and oxaliplatin (R-GemOx; solid line) or glofitamab, gemcitabine, and oxaliplatin (Glofit-GemOx; dashed line) in the updated analysis for the STARGLO trial. Patients administered Glofit-GemOx exhibited higher PFS (hazard ratio=0.40).

### DETAILED DESCRIPTION OF THE INVENTION

The invention provides methods for treating a patient having a CD20-positive cell proliferative disorder (e.g., a B cell proliferative disorder (e.g., non-Hodgkin's lymphoma (NHL) (e.g., a relapsed and/or refractory NHL, a diffuse-large B cell lymphoma (DLBCL) (e.g., a relapsed and/or refractory DLBCL), a follicular lymphoma (FL) (e.g., a relapsed and/or refractory FL or a transformed FL), or a mantle cell lymphoma (MCL) (e.g., a relapsed or refractory MCL)), or a central nervous system lymphoma (CNSL))) that includes administering to the patient glofitamab in combination with gemcitabine and oxaliplatin (Glofit-GemOx).

### (i) General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

### (ii) Definitions

Terms are used herein as generally used in the art, unless otherwise defined in the following.

It is to be understood that aspects and embodiments of the invention described herein include “comprising,” “consisting,” and “consisting essentially of” aspects and embodiments.

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. In some embodiments, reference to “about” a value or parameter herein refers to the value or parameter±10%.

The term “cluster of differentiation 20” or “CD20” as used herein, refers to any native CD20 from any vertebrate source, including mammals such as primates (e.g., humans) and rodents (e.g., mice and rats), unless otherwise indicated. CD20 (also known as B-lymphocyte antigen CD20, B-lymphocyte surface antigen B1, Leu-16, Bp35, BM5, and LF5; the human protein is characterized in UniProt database entry P11836) is a hydrophobic transmembrane protein with a molecular weight of approximately 35 kD expressed on pre-B and mature B lymphocytes (Valentine, M. A. et al., J. Biol. Chem. 264 (1989) 11282-11287; Tedder, T. F., et al., Proc. Natl. Acad. Sci. U.S.A. 85 (1988) 208-212; Stamenkovic, I., et al., J. Exp. Med. 167 (1988) 1975-1980; Einfeld, D. A., et al., EMBO J. 7 (1988) 711-717; Tedder, T. F., et al., J. Immunol. 142 (1989) 2560-2568). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. The term encompasses “full-length,” unprocessed CD20 as well as any form of CD20 that results from processing in the cell. The term also encompasses naturally occurring variants of CD20, e.g., splice variants or allelic variants. Alternative splicing of this gene results in two transcript variants which encode the same protein. In one embodiment, CD20 is human CD20.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD20 antibody binds to an epitope of CD20 that is conserved among CD20 from different species.

By “Type II anti-CD20 antibody” is meant an anti-CD20 antibody having binding properties and biological activities of Type II anti-CD20 antibodies as described in Cragg et al., Blood 103 (2004) 2738-2743; Cragg et al., Blood 101 (2003) 1045-1052, Klein et al., mAbs 5 (2013), 22-33, and summarized in Table 1 below.

TABLE 1
Properties of type I and type II anti-CD20 antibodies

| type I anti-CD20 antibodies | type Il anti-CD20 antibodies |
|---|---|
| Bind class I CD20 epitope | Bind class II CD20 epitope |
| Localize CD20 to lipid rafts | Do not localize CD20 to lipid rafts |
| High CDC * | Low CDC * |
| ADCC activity * | ADCC activity * |
| Full binding capacity to B cells | Approx. half binding capacity to B cells |
| Weak homotypic aggregation | Homotypic aggregation |
| Low cell death induction | Strong cell death induction |

* if IgG₁ isotype

Examples of type II anti-CD20 antibodies include, e.g., obinutuzumab (GA101), tositumumab (B1), humanized B-Ly1 antibody IgG₁ (a chimeric humanized IgG1 antibody as disclosed in WO 2005/044859), 11 B8 IgG₁ (as disclosed in WO 2004/035607) and AT80 IgG₁.

Examples of type I anti-CD20 antibodies include, e.g., rituximab, ofatumumab, veltuzumab, ocaratuzumab, ocrelizumab, PRO131921, ublituximab, HI47 IgG₃ (ECACC, hybridoma), 2C6 IgG₁ (as disclosed in WO 2005/103081), 2F2 IgG1 (as disclosed in WO 2004/035607 and WO 2005/103081) and 2H7 IgG₁ (as disclosed in WO 2004/056312).

“CD3” refers to any native CD3 from any vertebrate source, including mammals such as primates (e.g., humans), non-human primates (e.g., cynomolgus monkeys) and rodents (e.g., mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon subunit of human CD3 (CD3E). The amino acid sequence of human CD3ε is shown in UniProt (uniprot.org) accession no. P07766 (version 144), or NCBI (ncbi.nlm.nih.gov) RefSeq NP_000724.1. The amino acid sequence of cynomolgus monkey [Macaca fascicularis] CD3ε is shown in NCBI GENBANK® no. BAB71849.1.

Glofitamab is an anti-CD20/anti-CD3 bispecific antibody (WHO Drug Information (International Nonproprietary Names for Pharmaceutical Substances), Recommended INN: List 83, 2020, vol. 34, no. 1, p. 39; Proposed INN: List 121 WHO Drug Information, Vol. 33, No. 2, 2019, page 276, also known as CD20-TCB, RO7082859, or RG6026; CAS #: 2229047-91-8). Glofitamab is a novel T-cell-engaging bispecific (TCB) full-length antibody with a 2:1 molecular configuration for bivalent binding to CD20 on B cells and monovalent binding to CD3, particularly the CD3 epsilon chain (CD3ε), on T cells. Its CD3-binding region is fused to one of the CD20-binding regions in a head-to-tail fashion via a flexible linker. This structure endows glofitamab with superior in vitro potency versus other CD20-CD3 bispecific antibodies with a 1:1 configuration and leads to profound antitumor efficacy in preclinical DLBCL models. CD20 bivalency preserves this potency in the presence of competing anti-CD20 antibodies, providing the opportunity for pre- or co-treatment with these agents. Glofitamab comprises an engineered, heterodimeric Fc region with completely abolished binding to FcgRs and C1q. By simultaneously binding to human CD20-expressing tumor cells and to the CD3ε of the T-cell receptor (TCR) complex on T-cells, it induces tumor cell lysis, in addition to T-cell activation, proliferation and cytokine release. Lysis of B-cells mediated by glofitamab is CD20-specific and does not occur in the absence of CD20 expression or in the absence of simultaneous binding (cross-linking) of T-cells to CD20-expressing cells. In addition to killing, T-cells undergo activation due to CD3 cross-linking, as detected by an increase in T-cell activation markers (CD25 and CD69), cytokine release (IFNγ, TNFα, IL-2, IL-6, and IL-10), cytotoxic granule release (Granzyme B) and T-cell proliferation. The sequences of glofitamab are summarized in Table 2.

TABLE 2
Sequence IDs for glofitamab
Sequence IDs for glofitamab

| SEQ ID NO: | Description | SEQ ID NO: | Description |
|---|---|---|---|
| CD3 Heavy Chain | | CD3 Light Chain | |
| 9 | HVR-H1 (Kabat) | 12 | HVR-L1 (Kabat) |
| 10 | HVR-H2 (Kabat) | 13 | HVR-L2 (Kabat) |
| 11 | HVR-H3 (Kabat) | 14 | HVR-L3 (Kabat) |
| 15 | VH | 16 | VL |
| CD20 Heavy Chain | | CD20 Light Chain | |
| 1 | HVR-H1 (Kabat) | 4 | HVR-L1 (Kabat) |
| 2 | HVR-H2 (Kabat) | 5 | HVR-L2 (Kabat) |
| 3 | HVR-H3 (Kabat) | 6 | HVR-L3 (Kabat) |
| 7 | VH | 8 | VL |

Full-length antibody

| | | | |
|---|---|---|---|
| 17 | HC-knob | 18 | HC-hole |
| 19 | LC-CD3 | 20 | LC-CD20 |

As used herein, the term “release of cytokines” or “cytokine release” is synonymous with “cytokine storm” or “cytokine release syndrome” (abbreviated as “CRS”), and refers to an increase in the levels of cytokines, particularly tumor necrosis factor alpha (TNF-α), interferon gamma (IFN-γ), interleukin-6 (IL-6), interleukin-10 (IL-10), interleukin-2 (IL-2) and/or interleukin-8 (IL-8), in the blood of a patient during or shortly after (e.g., within 1 day of) administration of a therapeutic agent, resulting in adverse symptoms. Cytokine release is defined as a supraphysiologic response following administration of any immune therapy that results in activation or engagement of endogenous or infused T cells and/or other immune effector cells. Symptoms can be progressive, always include fever at the onset, and may include hypotension, capillary leak (hypoxia), and end-organ dysfunction (Lee et al. 2019). In some instances, e.g., after the administration of CAR-T cells, CRS can also occur several days after administration upon expansion of the CAR-T cells. The incidence and severity typically decrease with subsequent infusions. Symptoms may range from symptomatic discomfort to fatal events, and may include fever, chills, dizziness, hypertension, hypotension, dyspnea, restlessness, sweating, flushing, skin rash, tachycardia, tachypnea, headache, tumor pain, nausea, vomiting and/or organ failure.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor. Amino acid sequence deletions and insertions include amino- and/or carboxy-terminal deletions and insertions of amino acids. Particular amino acid mutations are amino acid substitutions. For the purpose of altering, e.g., the binding characteristics of an Fc region, non-conservative amino acid substitutions, i.e., replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g., 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc region to glycine can be indicated as 329G, G329, G₃₂₉, P329G, or Pro329Gly.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., receptor and a ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art. A particular method for measuring affinity is Surface Plasmon Resonance (SPR).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, the term “antigen binding moiety” refers to a polypeptide molecule that specifically binds to an antigenic determinant. In one embodiment, an antigen binding moiety is able to direct the entity to which it is attached (e.g., a cytokine or a second antigen binding moiety) to a target site, for example to a specific type of tumor cell or tumor stroma bearing the antigenic determinant. Antigen binding moieties include antibodies and fragments thereof as further defined herein. Preferred antigen binding moieties include an antigen binding domain of an antibody, comprising an antibody heavy chain variable region and an antibody light chain variable region. In certain embodiments, the antigen binding moieties may include antibody constant regions as further defined herein and known in the art. Useful heavy chain constant regions include any of the five isotypes: α, δ, ε, γ, or μ. Useful light chain constant regions include any of the two isotypes: κ and λ.

By “binds,” “specifically binds,” or is “specific for” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g., surface plasmon resonance technique (analyzed on a BIAcore instrument) (Liljeblad et al., Glyco J. 17, 323-329 (2000)), and traditional binding assays (Heeley, Endocr Res. 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

“Reduced binding,” for example reduced binding to an Fc receptor, refers to a decrease in affinity for the respective interaction, as measured for example by SPR. For clarity the term includes also reduction of the affinity to zero (or below the detection limit of the analytic method), i.e., complete abolishment of the interaction. Conversely, “increased binding” refers to an increase in binding affinity for the respective interaction.

As used herein, the term “antigen binding molecule” refers in its broadest sense to a molecule that specifically binds an antigenic determinant. Examples of antigen binding molecules are immunoglobulins and derivatives, e.g., fragments, thereof.

As used herein, the term “antigenic determinant” is synonymous with “antigen” and “epitope,” and refers to a site (e.g., a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, free in blood serum, and/or in the extracellular matrix (ECM). The proteins referred to as antigens herein (e.g., CD3) can be any native form the proteins from any vertebrate source, including mammals such as primates (e.g., humans) and rodents (e.g., mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g., splice variants or allelic variants. An exemplary human protein useful as antigen is CD3, particularly the epsilon subunit of CD3 (see UniProt no. P07766 (version 130), NCBI RefSeq no. NP_000724.1, for the human sequence; or UniProt no. Q95LI5 (version 49), NCBI GENBANK® no. BAB71849.1, for the cynomolgus [Macaca fascicularis] sequence). In certain embodiments a T cell activating bispecific antigen binding molecule described herein binds to an epitope of CD3 or a target cell antigen that is conserved among the CD3 or target cell antigen from different species.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN® (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX® operating system, including digital UNIX® V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen binding activity.

The terms “full length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv), and multispecific antibodies formed from antibody fragments. The term “antibody fragment” as used herein also encompasses single-domain antibodies. The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), ε (IgE), γ (IgG), or μ (IgM), some of which may be further divided into subclasses, e.g., γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen binding specificity.

Glutamine or glutamate residues at the N-terminus of antibody heavy or light chains may be converted to pyro-glutamate spontaneously (see e.g. Liu et al., Journal of Pharmaceutical Sciences. 97, 2426-2447 (2008), Rehder et al., Journal of Chromatography A. 1102, 164-175 (2006), Chelius et al., Anal Chem. 78, 2370-2376 (2006)). Hence, variable domains disclosed herein which comprise either a glutamine (Q) or a glutamate (E) amino acid residue at the N-terminus of an antibody heavy or light chain may comprise an N-terminal pyro-glutamate (pyroE) residue instead of the N-terminal Q or E residue. Likewise, antibody heavy chains or light chains disclosed herein which comprise either a glutamine (Q) or a glutamate (E) amino acid residue at the N-terminus, may comprise an N-terminal pyro-glutamate (pyroE) residue instead of the N-terminal Q or E residue. Accordingly, for each antibody heavy chain, light chain, or variable domain sequence disclosed herein that contains an N-terminal Q or E residue, the corresponding sequence with an N-terminal pyroE residue is also encompassed.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of

Proteins of Immunological Interest, Fifth Edition, NIH Publication 91-3242, Bethesda MD (1991), vols. 1-3.

In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term IgG “isotype” or “subclass” as used herein is meant any of the subclasses of immunoglobulins defined by the chemical and antigenic characteristics of their constant regions.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from cysteine 226 (C226), according to Kabat EU numbering, or from proline 230 (P230), according to Kabat EU numbering, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case in particular where the final two C-terminal amino acids of the heavy chain (e.g., monovalent heavy chain) are glycine (G448, Kabat EU numbering) and lysine (K449, Kabat EU numbering). Therefore, the C-terminal lysine (K449), or the C-terminal glycine (G448) and lysine (K449), of the Fc region may or may not be present. Alternatively, the final two C-terminal amino acids of the heavy chain (e.g., bivalent heavy chain) are glycine (G673, Kabat EU numbering) and lysine (K674, Kabat EU numbering). In this alternative, the C-terminal lysine (K674), or the C-terminal glycine (G673) and lysine (K674), of the Fc region may or may not be present. Amino acid sequences of heavy chains including an Fc region are denoted herein without C-terminal glycine-lysine dipeptide if not indicated otherwise. The corresponding sequence including a C-terminal glycine residue, or including a C-terminal glycine-lysine dipeptide, is also encompassed, however. Accordingly, in one aspect, a heavy chain (e.g., monovalent heavy chain) including an Fc region as specified herein comprises an additional C-terminal glycine-lysine dipeptide (G448 and K449, Kabat EU numbering), e.g., SEQ ID NO: 59. In one aspect, a heavy chain including an Fc region as specified herein comprises an additional C-terminal glycine residue (G448, Kabat EU numbering). In one aspect, a heavy chain (e.g., bivalent heavy chain) including an Fc region as specified herein comprises an additional C-terminal glycine-lysine dipeptide (G673 and K674, Kabat EU numbering), e.g., SEQ ID NO: 58. In one aspect, a heavy chain including an Fc region as specified herein comprises an additional C-terminal glycine residue (G673, Kabat EU numbering). Unless otherwise specified herein, numbering of amino acid residues in the Fc region or heavy chain constant region is according to the EU numbering system, also called the EU index, as described in Kabat 1991.

A “subunit” of an Fc domain as used herein refers to one of the two polypeptides forming the dimeric Fc domain, i.e., a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e., the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g., antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89).

The term “effector functions” when used in reference to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, the term “effector cells” refers to a population of lymphocytes that display effector moiety receptors, e.g., cytokine receptors, and/or Fc receptors on their surface through which they bind an effector moiety, e.g., a cytokine, and/or an Fc region of an antibody and contribute to the destruction of target cells, e.g., tumor cells. Effector cells may for example mediate cytotoxic or phagocytic effects. Effector cells include, but are not limited to, effector T cells such as CD8+ cytotoxic T cells, CD4+ helper T cells, γδ T cells, NK cells, lymphokine-activated killer (LAK) cells and macrophages/monocytes.

As used herein, the terms “engineer,” “engineered,” and “engineering,” are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. “Engineering”, particularly with the prefix “glyco-”, as well as the term “glycosylation engineering,” includes metabolic engineering of the glycosylation machinery of a cell, including genetic manipulations of the oligosaccharide synthesis pathways to achieve altered glycosylation of glycoproteins expressed in cells. Furthermore, glycosylation engineering includes the effects of mutations and cell environment on glycosylation. In one embodiment, the glycosylation engineering is an alteration in glycosyltransferase activity. In a particular embodiment, the engineering results in altered glucosaminyltransferase activity and/or fucosyltransferase activity. Glycosylation engineering can be used to obtain a “host cell having increased GnTIII activity” (e.g., a host cell that has been manipulated to express increased levels of one or more polypeptides having β(1,4)-N-acetylglucosaminyltransferase III (GnTIII) activity), a “host cell having increased ManII activity” (e.g., a host cell that has been manipulated to express increased levels of one or more polypeptides having α-mannosidase II (ManII) activity), or a “host cell having decreased α(1,6) fucosyltransferase activity” (e.g., a host cell that has been manipulated to express decreased levels of α(1,6) fucosyltransferase).

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate proteins used for the present invention. In one embodiment, the host cell is engineered to allow the production of an antibody with modified oligosaccharides. In certain embodiments, the host cells have been manipulated to express increased levels of one or more polypeptides having β(1,4)-N-acetylglucosaminyltransferase III (GnTIII) activity. In certain embodiments the host cells have been further manipulated to express increased levels of one or more polypeptides having α-mannosidase II (ManII) activity. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

As used herein, the term “polypeptide having GnTIII activity” refers to a polypeptide that is able to catalyze the addition of a N-acetylglucosamine (GlcNAc) residue in β-1,4 linkage to the β-linked mannoside of the trimannosyl core of N-linked oligosaccharides. This includes fusion polypeptides exhibiting enzymatic activity similar to, but not necessarily identical to, an activity of β(1,4)-N-acetylglucosaminyltransferase III, also known as β-1,4-mannosyl-glycoprotein 4-beta-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependency in a given activity as compared to the GnTIII (i.e., the candidate polypeptide will exhibit greater activity or not more than about 25-fold less and, preferably, not more than about ten-fold less activity, and most preferably, not more than about three-fold less activity relative to the GnTIII). In certain embodiments the polypeptide having GnTIII activity is a fusion polypeptide comprising the catalytic domain of GnTIII and the Golgi localization domain of a heterologous Golgi resident polypeptide. Particularly, the Golgi localization domain is the localization domain of mannosidase II or GnTI, most particularly the localization domain of mannosidase II. Alternatively, the Golgi localization domain is selected from the group consisting of: the localization domain of mannosidase I, the localization domain of GnTII, and the localization domain of α1,6 core fucosyltransferase. Methods for generating such fusion polypeptides and using them to produce antibodies with increased effector functions are disclosed in WO2004/065540, U.S. Provisional Pat. Appl. No. 60/495,142 and U.S. Pat. Appl. Publ. No. 2004/0241817, the entire contents of which are expressly incorporated herein by reference.

As used herein, the term “Golgi localization domain” refers to the amino acid sequence of a Golgi resident polypeptide which is responsible for anchoring the polypeptide to a location within the Golgi complex. Generally, localization domains comprise amino terminal “tails” of an enzyme.

As used herein, the term “polypeptide having ManII activity” refers to polypeptides that are able to catalyze the hydrolysis of the terminal 1,3- and 1,6-linked α-D-mannose residues in the branched GlcNAcMan₅GlcNAc₂ mannose intermediate of N-linked oligosaccharides. This includes polypeptides exhibiting enzymatic activity similar to, but not necessarily identical to, an activity of Golgi α-mannosidase II, also known as mannosyl oligosaccharide 1,3-1,6-α-mannosidase II (EC 3.2.1.114), according to the Nomenclature Committee of the International Union of Biochemistry and Molecular Biology (NC-IUBMB).

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

By “antibody having increased/reduced antibody dependent cell-mediated cytotoxicity (ADCC)” is meant an antibody having increased/reduced ADCC as determined by any suitable method known to those of ordinary skill in the art. One accepted in vitro ADCC assay is as follows:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/mL in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/mL;
   iii) 100 microliters of the final target cell suspension above are transferred to each well of a 96-well microtiter plate;
   iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (V/V) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector: target cell ratio of 25:1 and the plates are placed in an incubator under 5% CO₂ atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point v above), and SR is the average radioactivity quantified (see point ix above) for the SR controls (see point vi above);
4) “increased/reduced ADCC” is defined as either an increase/reduction in the maximum percentage of specific lysis observed within the antibody concentration range tested above, and/or a reduction/increase in the concentration of antibody required to achieve one half of the maximum percentage of specific lysis observed within the antibody concentration range tested above. The increase/reduction in ADCC is relative to the ADCC, measured with the above assay, mediated by the same antibody, produced by the same type of host cells, using the same standard production, purification, formulation and storage methods, which are known to those skilled in the art, but that has not been engineered.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

As used herein, the terms “first,” “second,” “third,” etc. with respect to antigen binding moieties or domains, are used for convenience of distinguishing when there is more than one of each type of moiety or domain. Use of these terms is not intended to confer a specific order or orientation unless explicitly so stated.

The terms “multispecific” and “bispecific” mean that the antigen binding molecule is able to specifically bind to at least two distinct antigenic determinants. Typically, a bispecific antigen binding molecule comprises two antigen binding sites, each of which is specific for a different antigenic determinant. In certain embodiments, a bispecific antigen binding molecule is capable of simultaneously binding two antigenic determinants, particularly two antigenic determinants expressed on two distinct cells.

The term “valent” or “valency” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e., one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

An “activating T cell antigen” as used herein refers to an antigenic determinant expressed by a T lymphocyte, particularly a cytotoxic T lymphocyte, which is capable of inducing or enhancing T cell activation upon interaction with an antigen binding molecule. Specifically, interaction of an antigen binding molecule with an activating T cell antigen may induce T cell activation by triggering the signaling cascade of the T cell receptor complex. An exemplary activating T cell antigen is CD3. In a particular embodiment the activating T cell antigen is CD3, particularly the epsilon subunit of CD3 (see UniProt no. P07766 (version 130), NCBI RefSeq no. NP_000724.1, for the human sequence; or UniProt no. Q95LI5 (version 49), NCBI GENBANK® no. BAB71849.1, for the cynomolgus [Macaca fascicularis] sequence).

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating therapeutic agents used in the present invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma. In a particular embodiment, the target cell antigen is CD20, particularly human CD20 (see UniProt no. P11836).

A “B-cell antigen” as used herein refers to an antigenic determinant presented on the surface of a B lymphocyte, particularly a malignant B lymphocyte (in that case the antigen also being referred to as “malignant B-cell antigen”).

A “T-cell antigen” as used herein refers to an antigenic determinant presented on the surface of a T lymphocyte, particularly a cytotoxic T lymphocyte.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g., a Fab molecule and an Fc domain subunit) are linked by peptide bonds, either directly or via one or more peptide linkers.

An “effective amount” of an agent refers to the amount that is necessary to result in a physiological change in the cell or tissue to which it is administered.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

By “therapeutic agent” is meant an active ingredient, e.g., of a pharmaceutical composition, that is administered to a patient in an attempt to alter the natural course of a disease in the patient being treated, and can be performed either for prophylaxis or during the course of clinical pathology. An “immunotherapeutic agent” refers to a therapeutic agent that is administered to a patient in an attempt to restore or enhance the patient's immune response, e.g., to a tumor.

The term “pharmaceutical composition” refers to a preparation which is in such form as to permit the biological activity of an active ingredient contained therein to be effective, and which contains no additional components which are unacceptably toxic to a patient to which the composition would be administered.

A “pharmaceutically acceptable carrier” refers to an ingredient in a pharmaceutical composition, other than an active ingredient, which is nontoxic to a patient. A pharmaceutically acceptable carrier includes, but is not limited to, a buffer, excipient, stabilizer, or preservative.

The term “package insert” or “instructions for use” is used to refer to instructions customarily included in commercial packages of therapeutic products that contain information about the indications, usage, dosage, administration, combination therapy, contraindications and/or warnings concerning the use of such therapeutic products.

The term “combination treatment” noted herein encompasses combined administration (where two or more therapeutic agents are included in the same or separate formulations), and separate administration, in which case, administration of an antibody as reported herein can occur prior to, simultaneously, and/or following, administration of the additional therapeutic agent or agents, preferably an antibody or antibodies.

By a “crossover” Fab molecule (also termed “Crossfab”) is meant a Fab molecule wherein the variable domains or the constant domains of the Fab heavy and light chain are exchanged (i.e., replaced by each other), i.e., the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e., comprising a heavy chain composed of the heavy chain variable and constant domains (VH-CH1, in N- to C-terminal direction), and a light chain composed of the light chain variable and constant domains (VL-CL, in N- to C-terminal direction).

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term “nucleic acid molecule” refers to any one or more nucleic acid segments, e.g., DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98%, or 99% identical to a nucleotide sequence of the present invention can be determined conventionally using known computer programs, such as the ones discussed above for polypeptides (e.g., ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” Is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The term “about” as used herein refers to the usual error range for the respective value readily known to the skilled person in this technical field. Reference to “about” a value or parameter herein includes (and describes) embodiments that are directed to that value or parameter per se.

By “B cell proliferative disorder” is meant a disease wherein the number of B cells in a patient is increased as compared to the number of B cells in a healthy patient, and particularly wherein the increase in the number of B cells is the cause or hallmark of the disease. A “CD20-positive B cell proliferative disorder” is a B cell proliferative disorder wherein B-cells, particularly malignant B-cells (in addition to normal B-cells), express CD20.

Exemplary B cell proliferation disorders include Non-Hodgkin lymphoma (NHL), diffuse large B-cell lymphoma (DLBCL; e.g., relapsed or refractory DLBCL not otherwise specified (NOS), high grade B cell lymphoma (HGBCL; e.g., HGBCL NOS, double-hit HGBCL, and triple-hit HGBCL), primary mediastinal large B-cell lymphoma (PMBCL), and DLBCL arising from FL (transformed FL; trFL)); follicular lymphoma (FL), including Grades 1-3b FL; mantle-cell lymphoma (MCL); and marginal zone lymphoma (MZL), including splenic, nodal or extra-nodal MZL. In one embodiment, the CD20-positive B cell proliferative disorder is a Burkitt lymphoma (BL); a Burkitt leukemia (BAL; mature B-cell leukemia FAB L3); DLBCL, or PMBCL. In one embodiment the CD20-positive B cell proliferative disorder is a relapsed or refractory NHL (e.g., a relapsed or refractory DLBCL, a relapsed or refractory FL, or a relapsed or refractory MCL). In one embodiment, the BL, BAL, DLBCL, or PMBCL is relapsed and/or refractory. In one embodiment, the BL, BAL, DLBCL, or PMBCL has relapsed after or is refractory to a first-line standard-of-care chemoimmunotherapy.

“Refractory disease” is defined as failure to achieve complete remission to first-line therapy, including defined as failure to achieve complete remission to first-line therapy, including:
Progressive Disease (PD) as best response to first-line therapy.
Stable disease (SD) as best response after at least 4 cycles of first-line therapy (e.g., 4 cycles of R-CHOP).
Partial Response (PR) as best response after at least 6 cycles and with either biopsy-proven residual disease or subsequent disease progression.

“Relapsed disease” is defined as disease relapse or recurrence after complete remission to first-line therapy. In one embodiment disease relapse is proven by biopsy. In one embodiment, patients have relapsed after or failed to respond to at least two prior systemic treatment regimens (including at least one prior regimen containing anthracycline, and at least one containing an anti CD20-directed therapy).

An “individual,” “patient,” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the individual, patient, or subject is a human. In one instance, each patient in a population of patients is human. In one instance, each patient in a reference population of patients is human. In one embodiment, a patient is considered to be a pediatric patient if the pediatric patient is younger than 18 years old (i.e., aged 17 years or less). In one embodiment, the pediatric patient is aged between 6 months and 17 years. In one embodiment, a patient is considered to be a young adult patient if the young adult patient is aged between 18 years and 30 years. In one embodiment, a patient is considered to be an adult patient if the patient is aged 18 years old or more.

A “transplant eligible” patient or a patient “eligible for autologous stem cell transplantation (SCT)” is a patient who meets eligibility for, who is recommended for or who can receive, autologous SCT. In one embodiment, “transplant eligible” is defined as being medically eligible for intensive platinum-based salvage therapy followed by autologous stem cell transplantation (ASCT). In one embodiment the transplant eligible patient achieves an objective response as well as mobilization of the target dose of at least 2,000,000 CD34+ hematopoietic stem cells/kg. In one embodiment, “transplant eligible” is defined as being medically eligible for two to three cycles of salvage therapy with R-ICE and glofitamab to achieve CR followed by allogeneic or autologous hematopoietic stem cell transplantation (HSCT).

A “CAR-T cell therapy eligible” patient or a patient “eligible for CAR-T cell therapy” is a patient who meets eligibility for, who is recommended for or who can receive, chimeric antigen receptor (CAR) T-cell therapy.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods of the invention are used to delay development of a disease or to slow the progression of a disease. In one embodiment, the disease being treated is a CD20-positive B cell proliferative disorder, e.g., a Burkitt lymphoma (BL); a Burkitt leukemia (BAL; mature B-cell leukemia FAB L3); DLBCL, or PMBCL.

As used herein, “delaying progression” of a disorder or disease means to defer, hinder, slow, retard, stabilize, and/or postpone development of the disease or disorder (e.g., a CD20-positive B cell proliferative disorder, e.g., NHL, e.g., DLBCL, e.g., Burkitt lymphoma (BL); e.g., Burkitt leukemia (BAL; mature B-cell leukemia FAB L3), or e.g., PMBCL). This delay can be of varying length of time, depending on the history of the disease and/or individual being treated. As is evident to one skilled in the art, a sufficient or significant delay can, in effect, encompass prevention, in that the individual does not develop the disease. For example, in a late-stage cancer, development of central nervous system (CNS) metastasis, may be delayed.

By “reduce” or “inhibit” is meant the ability to cause an overall decrease, for example, of 20%, 30%, 40%, 50%, 60%, 70%, 75%, 80%, 85%, 90%, 95%, or greater. For clarity the term includes also reduction to zero (or below the detection limit of the analytical method), i.e., complete abolishment or elimination. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with glofitamab using the step-up dosing regimen of the invention relative to unchanging, preset dosing with the target dose of the bispecific antibody. In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments reduce or inhibit can refer to the symptoms of the CD20-positive B cell proliferative disorder being treated (e.g., an NHL (e.g., a DLBCL), an FL (e.g., a relapsed and/or refractor FL or a transformed FL), an MCL, a high-grade B cell lymphoma, or a PMLBCL), the presence or size of metastases, or the size of the primary tumor. In one embodiment, the CD20-positive B cell proliferative disorder being treated is a Burkitt lymphoma (BL); a Burkitt leukemia (BAL; mature B-cell leukemia FAB L3); DLBCL, or PMBCL.

As used herein, “administering” is meant a method of giving a dosage of a compound (e.g., glofitamab) or a composition (e.g., a pharmaceutical composition, e.g., a pharmaceutical composition including glofitamab) to a patient. The compounds and/or compositions utilized in the methods described herein can be administered intravenously (e.g., by intravenous infusion).

A “fixed” or “flat” dose of a therapeutic agent (e.g., glofitamab) herein refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

A “target dose” herein refers to the dose of glofitamab that achieves therapeutic effect, i.e., achieves the desired clinical efficacy. It was found that for glofitamab a possible target dose is 16 mg or 30 mg.

An “unchanging or preset dosing with target dose” and a “treatment regimen without a step-up dosing regimen” refers to a dosing schedule that uses the same dosage in the first and second cycle and optionally also any subsequent treatment cycle, as opposed to step-up dosing, which uses lower dosages in the first few treatment cycles and only reaches the target dose in the second or in a later treatment cycle.

The terms “treatment cycle” or “cycle” (abbreviated: “C”) as used herein mean a course of one or more doses of glofitamab that is repeated on a regular schedule, optionally with periods of rest (no treatment) in between. In one aspect of the invention, the first treatment cycle comprises a first and a second dose of glofitamab, followed by a period of rest. In one such embodiment, the first treatment cycle comprises a first dose of glofitamab on Day 8 of the first cycle, and a second dose of glofitamab on Day 15 of the first cycle, followed by 6 days of rest. In one embodiment the second and any subsequent cycles comprise one dose of glofitamab given at Day 8 of that cycle, followed by 13 days of rest. In one embodiment, one treatment cycle comprises 21 days. In another embodiment, one treatment cycle comprises 14 days. The treatment schedule according to the invention may comprise 2 or more treatment cycles, in particular 3 treatment cycles. In some embodiments, a treatment cycle is referred to as a “dosing cycle.”

“Individual response” or “response” can be assessed using any endpoint indicating a benefit to the patient, including, without limitation, (1) inhibition, to some extent, of disease progression (e.g., progression of a CD20-positive B cell proliferative disorder, e. g., a non-Hodgkin's lymphoma (NHL)); including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the CD20-positive B cell proliferative disorder, e.g., a B cell proliferative disorder; (6) increase or extend in the length of survival, including overall survival and progression-free survival; and/or (7) decreased mortality at a given point of time following treatment.

As used herein, “complete response,” “CR,” or “complete remission” refers to disappearance of all target lesions. In one embodiment standard NHL response criteria are assessed for determining CR. (Lugano Classification, Cheson et al. J Clin Oncol. 2014 Sep. 20; 32 (27): 3059-3067.). In one embodiment the CR rate is defined as the proportion of patients whose overall response is a CR on positron emission tomography/computed tomography (PET/CT), as determined by the investigator according to Lugano criteria. In one embodiment CR is defined as complete metabolic response as determined by PET/CT of the lymph nodes and extra-lymphatic sites, with a score of 1, 2, or 3 with or without a residual mass on 5PS, wherein PET 5PS: 1=no uptake above background; 2=uptake >mediastinum; 3=uptake>mediastinum but ≤liver; 4=uptake moderately >liver; 5=uptake markedly higher than liver and/or new lesions; X=new areas of uptake unlikely to be related to lymphoma. In one embodiment, CR is defined as complete radiologic response as determined by CT of the lymph nodes and extra-lymphatic sites, wherein the target nodes/nodal masses must regress to ≤1.5 cm in LDi (longest transverse diameter of a lesion) and no extralymphatic sites of disease remain.

As used herein, “partial response” or “PR” refers to Partial metabolic response as determined by PET/CT of the lymph nodes and extra-lymphatic sites and/or Partial remission as determined by CT of the lymph nodes and extra-lymphatic sites. In one embodiment, Partial metabolic response is defined by a score 4 or 5 b with reduced uptake compared with baseline and residual mass(es) of any size as determined by PET/CT of the lymph nodes and extra-lymphatic sites, wherein PET 5PS: 1=no uptake above background; 2=uptake >mediastinum; 3=uptake>mediastinum but ≤liver; 4=uptake moderately >liver; 5=uptake markedly higher than liver and/or new lesions; X=new areas of uptake unlikely to be related to lymphoma. In one embodiment partial remission is defined as at least a 50% decrease in the product of the diameters (SPD) of up to 6 target measurable nodes and extranodal sites, taking as reference the baseline SPD. For pediatric patients (<18 years old), PR is assessed using the International Pediatric NHL Response Criteria (Sandlund J T, Guillerman R P, Perkins S L, et al.

International pediatric non-Hodgkin lymphoma response criteria. J. Clin. Oncol. 33:2106-2111, 2015). An “effective response” of a patient or a patient's “responsiveness” to treatment with a medicament and similar wording refers to the clinical or therapeutic benefit imparted to a patient as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

“Duration of complete response” (DOCR) is defined as the time from the first occurrence of a documented complete response to disease progression or death from any cause (whichever occurs first), as determined by the investigator according to Lugano criteria (Cheson et al. J Clin Oncol. 2014 Sep. 20; 32 (27): 3059-3067.). CR is assessed by the investigator, using the International Pediatric NHL Response Criteria for pediatric patients <18 years old (Sandlund J T, Guillerman R P, Perkins S L, et al. International pediatric non-Hodgkin lymphoma response criteria. J. Clin. Oncol. 33:2106-2111, 2015).

“Duration of objective response” (DOR), defined as the time from the first occurrence of a documented objective response (CR or PR) to disease progression or death from any cause (whichever occurs first), as determined by the investigator according to Lugano criteria (Cheson et al. J Clin Oncol. 2014 Sep. 20; 32 (27): 3059-3067.). CR and/or PR is assessed by the investigator, using the International Pediatric NHL Response Criteria for pediatric patients <18 years old (Sandlund J T, Guillerman R P, Perkins S L, et al. International pediatric non-Hodgkin lymphoma response criteria. J. Clin. Oncol. 33:2106-2111, 2015).

“Progression-free survival” (PFS) is defined as the time from the first treatment with glofitamab to the first occurrence of disease progression or death from any cause, whichever occurs first. In one embodiment, PFS is assessed based on the Lugano Classification (Cheson et al. J Clin Oncol. 2014 Sep. 20; 32 (27): 3059-3067.). PFS is assessed by the investigator, using the International Pediatric NHL Response Criteria for pediatric patients <18 years old (Sandlund J T, Guillerman R P, Perkins S L, et al. International pediatric non-Hodgkin lymphoma response criteria. J. Clin. Oncol. 33:2106-2111, 2015).

“Overall survival” (OS) is defined as time from the first treatment with glofitamab to the date of death from any cause.

As used herein “Event-free survival” (EFS) is defined as the time from the first treatment with the glofitamab the first occurrence of disease progression as determined by the investigator according to Lugano criteria, initiation of new anti-lymphoma therapy (not including planned ASCT), or death from any cause (whichever occurs first

As used herein, “objective response rate” (ORR) is defined as the sum of partial response (PR) rate and complete response (CR) rate. In one embodiment, ORR is evaluated based on the Lugano Classification (Cheson et al. J Clin Oncol. 2014 Sep. 20; 32 (27): 3059-3067). In one embodiment, the ORR is defined as the proportion of participants that achieves a CR or PR within three cycles of glofitamab+GemOx treatment regimen described therein.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD, or at least a 50% increase in the SPD of target legions, taking as reference the smallest SPD, recorded since the treatment started or the presence of one or more new lesions.

As used herein, an “infusion-related reaction,” “IRR,” or infusion-related adverse event” is an adverse event that occurs in a patient or subject during or within 24 hours after administration of a drug (e.g., glofitamab; or an anti-CD20 antibody, e.g., obinutuzumab or rituximab). IRRs may be graded as Grades 1-5 according to, e.g., NCI CTCAE v.4.

“Mobilization-adjusted response rate (MARR)” is defined as the percentage of patients who achieve an objective response as well as mobilization of the target dose of 2,000,000 CD34+ hematopoietic stem cells/kg typically required as a minimum for ASCT.

As used herein, the term “GemOx” refers to gemcitabine plus oxaliplatin.

The term “corticosteroid prophylaxis” as used herein refers to an optimized, increased regimen of steroids for the treatment of patients with glofitamab to optimize the CRS profile of this treatment (e.g., to reduce the incidence of CRS events of in a patient population treated with glofitamab, to reduce the likelihood of a CRS event in a patient treated with glofitamab, or to reduce the seriousness (e.g., the Grade) of a CRS event or CRS events), wherein the corticosteroids are administered prior to and after administration of glofitamab. In one embodiment, the corticosteroids are administered for the first two step up doses (2.5 mg and 10 mg dose) of glofitamab in cycle 1. In one embodiment, the corticosteroids are administered about one day prior to, on the same day and one day after administration of glofitamab. In one embodiment, the corticosteroid prophylaxis is administered about 30 hours, about 29 hours, about 28 hours, about 27 hours, about 26 hours, about 25 hours, about 24 hours, about 23 hours, about 22 hours, about 21 hours, about 20 hours, about 19 hours, about 18 hours prior to administration of glofitamab. In one embodiment the corticosteroid prophylaxis is administered about 30-90 minutes or about 60 minutes prior to administration of glofitamab. In one embodiment the corticosteroid prophylaxis is administered about 25, about 30, about 40, about 50, about 60, about 70, about 80, about 90, or about 100 minutes prior to administration of glofitamab. In one embodiment, the corticosteroid prophylaxis is administered about 24 hours after administration of glofitamab. In one embodiment, the corticosteroid prophylaxis is administered about 30 hours, about 29 hours, about 28 hours, about 27 hours, about 26 hours, about 25 hours, about 24 hours, about 23 hours, about 22 hours, about 21 hours, about 20 hours, about 19 hours, about 18 hours after administration of glofitamab.

(iii) Combination Treatment of Glofitamab and GemOx

The invention provides methods for treating a patient having a CD20-positive cell proliferative disorder (e.g., a B cell proliferative disorder (e.g., non-Hodgkin's lymphoma (NHL) (e.g., a relapsed and/or refractory NHL, a diffuse-large B cell lymphoma (DLBCL) (e.g., a relapsed and/or refractory DLBCL), a follicular lymphoma (FL) (e.g., a relapsed and/or refractory FL or a transformed FL), or a mantle cell lymphoma (MCL) (e.g., a relapsed or refractory MCL)), or a central nervous system lymphoma (CNSL))) that includes administering to the patient glofitamab in combination with GemOx.

In some instances, the present methods are used for treating a patient having relapsed and/or refractory NHL (e.g., an aggressive NHL (e.g., a relapsed and/or refractory DLBCL, a relapsed and/or refractory FL, or a relapsed and/or refractory MCL)). In some instances, the patient has relapsed following one or more (e.g., one, two, three, four, five, or more) prior therapies (e.g., one or more prior systemic therapies, e.g., one or more prior systemic chemotherapies (e.g., one or more prior systemic therapies involving administration of anthracycline), one or more prior stem cell therapies, or one or more prior CAR-T cell therapies) after having a documented history of response (e.g., a complete response or a partial response) of at least 6 months in duration from completion of the therapy. In some instances, the patient is refractory to any prior therapy (e.g., has had no response to the prior therapy, or progression within 6 months of completion of the last dose of therapy). Thus, in some embodiments, the present dosing regimen is a second-line therapy. In some embodiments, the present dosing regimen is a third-line therapy. In some embodiments, the patient has a transformed FL, and/or is refractory to standard therapies for transformed FL. In some embodiments, the FL is a Graded FL (e.g., a Grade 1, 2, 3a, or 3b FL). In some embodiments, the DLBCL is DLBCL NOS.

In some embodiments, the patient is not a candidate for stem cell transplantation (e.g., ineligible for stem cell transplantation). In some embodiments, the patient is ineligible for hematopoietic stem cell transplantation (HSCT) (e.g., ineligible for HSCT). In some embodiments, the patient is ineligible for autologous stem cell transplantation (ASCT) (e.g., ineligible for ASCT).

In some embodiments, the patient has relapsed after or is refractory to one prior line of therapy and is not a candidate for HSCT, and the patient: (a) has a left ventricular ejection fraction ≤40%, (b) has creatinine clearance or glomerular filtration rate ≤45 mL/min, (c) has Eastern Cooperative Oncology Group (ECOG) Performance Status of ≥2, (d) is aged 70 years or more, (e) refused high-dose chemotherapy and/or transplant, and/or (f) had insufficient response to pre-transplant chemotherapy to be able to proceed to transplant

In one aspect, the invention features a method of treating relapsed or refractory diffuse large B-cell lymphoma (DLBCL) in a human patient in need thereof, comprising administering to the human patient an effective amount of:
(a) glofitamab
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
in the absence of glofitamab.

In one aspect, the invention features a method for treating relapsed or refractory diffuse large B-cell lymphoma (DLBCL) in a human patient in need thereof, comprising administering to the human patient an effective amount of:
(a) glofitamab
(b) gemcitabine, and
(c) oxaliplatin,
   wherein the patient has relapsed after or is refractory to one prior line of therapy and is not a candidate for HSCT, and wherein the patient:
(a) has a left ventricular ejection fraction ≤40%,
(b) has creatinine clearance or glomerular filtration rate ≤45 mL/min,
(c) has Eastern Cooperative Oncology Group (ECOG) Performance Status of ≥2,
(d) is aged 70 years or more,
(e) refused high-dose chemotherapy and/or transplant, and/or
(f) had insufficient response to pre-transplant chemotherapy to be able to proceed to transplant.

In one aspect, the invention features a method for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein the DLBCL is a DLBCL not otherwise specified (DLBCL NOS), and wherein the patient is not a candidate for hematopoietic stem cell transplantation (HSCT).

In one aspect, the invention features a method for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein the DLBCL is a DLBCL not otherwise specified (DLBCL NOS), and wherein the patient is not a candidate for hematopoietic stem cell transplantation (HSCT).

In one aspect, the invention features a method for treating relapsed or refractory diffuse large B-cell lymphoma not otherwise specified (DLBCL NOS) in a human patient in need thereof, wherein the patient is not a candidate for hematopoietic stem cell transplantation (HSCT), and wherein the method comprises administering an effective amount of glofitamab, gemcitabine, and oxaliplatin.

In one aspect, the invention features a method for treating relapsed or refractory diffuse large B-cell lymphoma not otherwise specified (DLBCL NOS) in a human patient in need thereof, wherein the patient is not a candidate for hematopoietic stem cell transplantation (HSCT), wherein the patient is aged 18 years or more, and wherein the method comprises administering an effective amount of glofitamab, gemcitabine, and oxaliplatin.

In one aspect, the invention features a method for treating relapsed or refractory diffuse large B-cell lymphoma not otherwise specified (DLBCL NOS) in an adult human patient in need thereof, wherein the patient is not a candidate for hematopoietic stem cell transplantation (HSCT), and wherein the method comprises administering an effective amount of glofitamab, gemcitabine, and oxaliplatin. In one embodiment, an adult human patient is aged 18 years or more.

In one embodiment, the PFS or the reference PFS is measured starting from the time from randomization to the time of a first occurrence of disease progression or death from any cause.

In one embodiment, the PFS or the reference PFS is the median PFS of the plurality of human patients receiving the corresponding treatment.

In one embodiment, the improvement in PFS is statistically significant.

In one embodiment, the improvement of the median PFS is an increase in the PFS compared to the reference PFS of at least 1 month (e.g., at least 2, 3, 4, 5, 6, 7, 8, 9, 10, 11, 12, 13, 14, 15, 20, 25, 30, or more months).

In one embodiment, the improvement of the median PFS is an increase in the PFS compared to the reference PFS of between 1 and 18 months (e.g., between 1 and 8 months, between 8 and 18 months, between 5 and 15 months, or between 6 and 10 months; e.g., about 1, 2, 3, 4, 5, 6, 7, 8, 9, 10, 11, 12, 13, 14, 15, 16, 17, or 18 months).

In one embodiment, the improvement of the median PFS is an increase in the PFS compared to the reference PFS of about 9 months. In one embodiment, the improvement of the median PFS is an increase in the PFS compared to the reference PFS of about 10 months.

In one embodiment, administering such treatment to a plurality of human patients results in a statistically significant improvement in the PFS as compared to the control treatment with: a hazard ratio of no more than 0.2, 0.3, 0.4, 0.5, 0.6, or 0.7.

In one embodiment, administering such treatment to a plurality of human patients results in a statistically significant improvement in the PFS as compared to the control treatment with: a hazard ratio of about 0.1, 0.2, 0.3, 0.4, 0.5, 0.6, or 0.7. In one embodiment, the hazard ratio is about 0.42. In one embodiment, the hazard ratio is about 0.42 (95% confidence interval: 0.29, 0.61). In one embodiment, the hazard ratio is about 0.40. In one embodiment, the hazard ratio is about 0.40 (95% confidence interval: 0.28, 0.57). In one embodiment, administering such treatment to a plurality of human patients results in a statistically significant improvement in the PFS as compared to the control treatment with a hazard ratio of about 0.41 (95% confidence interval: 0.29, 0.58).

In one embodiment, the hazard ratio is a stratified hazard ratio.

In one embodiment, administering such treatment to a plurality of human patients results in an increase in the rate of PFS compared to the rate of reference PFS at 6 months of at least 5% (e.g., at least 10%, 15%, 20%, 25%, 30%, 35%, 40%, 45%, 50%, 55%, 60%, or more).

In one embodiment, administering such treatment to a plurality of human patients results in an increase in the rate of PFS compared to the rate of reference PFS at 6 months of between 5% and 45% (e.g., between 5% and 25%, between 25% and 45%, between 10% and 40%, or e.g., between 20% and 30%; e.g., about 5%, 10%, 15%, 20%, 25%, 30%, 35%, 40%, or 45%).

In one embodiment, administering such treatment to a plurality of human patients results in an increase in the rate of PFS compared to the rate of reference PFS at 6 months of about 25%.

In one embodiment, administering such treatment to a plurality of human patients results in an increase in the rate of PFS compared to the rate of reference PFS at 12 months of at least 5% (e.g., at least 10%, 15%, 20%, 25%, 30%, 35%, 40%, 45%, 50%, 55%, 60%, or more).

In one embodiment, administering such treatment to a plurality of human patients results in an increase in the rate of PFS compared to the rate of reference PFS at 12 months of between 5% and 45% (e.g., between 5% and 25%, between 25% and 45%, between 10% and 40%, or e.g., between 20% and 30%; e.g., about 5%, 10%, 15%, 20%, 25%, 30%, 35%, 40%, or 45%).

In one embodiment, administering such treatment to a plurality of human patients results in an increase in the rate of PFS compared to the rate of reference PFS at 12 months of about 25%.

In one embodiment, administering such treatment to a plurality of human patients results in an improvement of the complete response rate (CR rate), objective response rate (ORR), duration of objective response, and/or duration of CR (DOCR) as compared to the control treatment.

In one embodiment, the CR rate is the proportion of patients whose best overall response is a CR on PET/computed tomography (CT).

In one embodiment, the improvement of the CR rate is an increase of at least 15% (e.g., at least 20%, 25%, 30%, 35%, 40%, 45%, 50%, 55%, 60%, or more).

In one embodiment, the improvement of the CR rate is an increase of between 15% and 50% (e.g., between 20% and 30%, between 30% and 45%, between 25% and 40%, e.g., between 25% and 35%, or between 20% and 45%; e.g., about 20%, 25%, 30%, 35%, 40%, or 45%).

In one embodiment, the improvement of the CR rate is an increase of about 30%. In one embodiment, the improvement of the CR rate is an increase of about 33%.

In one embodiment, the ORR is the proportion of patients whose best overall response is a partial response (PR) or a CR.

In one embodiment, the DOCR is measured as the time from the first occurrence of a documented objective response (CR or PR) to disease progression, or death from any cause, whichever occurs first.

In one embodiment, the improvement in the DOCR is an increase of at least 2 months (e.g., at least 3, 4, 5, 6, 7, 8, 9, 10, 11, 12, 13, 14, 15, 20, 25, 30, or more months).

In one embodiment, the improvement in the DOCR is an increase of between 2 and 20 months (e.g., between 2 and 8 months, between 8 and 20 months, between 5 and 15 months, or between 6 and 10 months; e.g., about 2, 3, 4, 5, 6, 7, 8, 9, 10, 11, 12, 13, 14, 15, 16, 17, 18, 19, or 20 months).

In one embodiment, the improvement in the DOCR is an increase of about 8 months.

In one embodiment, the duration of CR is measured as the time from the first occurrence of a documented CR to disease progression, or death from any cause, whichever occurs first.

In one aspect, a method for treating relapsed or refractory diffuse large B-cell lymphoma (DLBCL) in a human patient in need thereof is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS,
   wherein the reference OS is the OS of a plurality of human patients who have received a control treatment comprising:
(a) rituximab,
(b) gemcitabine, and
(c) oxaliplatin,
in the absence of glofitamab.

In one embodiment, the OS or the reference OS is measured starting from the time from randomization to death from any cause.

In one embodiment, the OS or the reference OS is the median OS of the plurality of human patients receiving the corresponding treatment.

In one embodiment, the improvement in OS is statistically significant.

In one embodiment, the improvement of the median OS is an increase in the OS compared to the reference OS of at least 1 month (e.g., at least 2, 3, 4, 5, 6, 7, 8, 9, 10, 11, 12, 13, 14, 15, 20, 25, 30, 35, 40, 45, 50, or more months).

In one embodiment, the improvement of the median OS is an increase in the OS compared to the reference OS of between 1 and 30 months (e.g., between 1 and 9 months, between 9 and 30 months, between 5 and 15 months, or between 5 and 25 months; e.g., about 1, 2, 3, 4, 5, 6, 7, 8, 9, 10, 11, 12, 13, 14, 15, 16, 17, 18, 19, 20, 25, or 30 months).

In one embodiment, the improvement of the median OS is an increase in the OS compared to the reference OS of about 13 months.

In one embodiment, administering such treatment to a plurality of human patients results in a statistically significant improvement in the OS as compared to the control treatment with a hazard ratio of no more than 0.3, 0.4, 0.5, 0.6, 0.7, or 0.8.

In one embodiment, administering such treatment to a plurality of human patients results in a statistically significant improvement in the OS as compared to the control treatment with a hazard ratio of about 0.3, 0.4, 0.5, 0.6, 0.7, or 0.8. In one embodiment, the hazard ratio is about 0.62. In one embodiment, the hazard ratio is about 0.62 (95% confidence interval: 0.43, 0.88). In some embodiments, administering such treatment to a plurality of human patients results in a statistically significant improvement in the OS as compared to the control treatment with a hazard ratio of about 0.60 (95% confidence interval: 0.42, 0.85).

In one embodiment, the hazard ratio is a stratified hazard ratio.

In one embodiment, administering such treatment to a plurality of human patients results in an increase in the rate of OS compared to the rate of reference OS at 12 months of at least 5% (e.g., at least 10%, 15%, 20%, 25%, 30%, 35%, 40%, 45%, 50%, 55%, or more).

In one embodiment, administering such treatment to a plurality of human patients results in an increase in the rate of OS compared to the rate of reference OS at 12 months of between 5% and 30% (e.g., between 5% and 10%, between 10% and 30%, between 10% and 20%, or between 5% and 15%; e.g., about 5%, 10%, 15%, 20%, 25%, or 30%).

In one embodiment, administering such treatment to a plurality of human patients results in an increase in the rate of OS compared to the rate of reference OS at 12 months of about 10%.

In one embodiment, administering such treatment to a plurality of human patients results in an increase in the rate of OS compared to the rate of reference OS at 18 months of at least 5% (e.g., at least 10%, 15%, 20%, 25%, 30%, 35%, 40%, 45%, 50%, 55%, or more).

In one embodiment, administering such treatment to a plurality of human patients results in an increase in the rate of OS compared to the rate of reference OS at 18 months of between 5% and 35% (e.g., between 5% and 20%, between 20% and 35%, between 10% and 30%, or between 15% and 25%; e.g., about 5%, 10%, 15%, 20%, 25%, 30%, or 35%).

In one embodiment, administering such treatment to a plurality of human patients results in an increase in the rate of OS compared to the rate of reference OS at 18 months of about 20%.

In one embodiment, administering such treatment to a plurality of human patients results in an increase in the rate of OS compared to the rate of reference OS at 24 months of at least 5% (e.g., at least 10%, 15%, 20%, 25%, 30%, 35%, 40%, 45%, 50%, 55%, or more).

In one embodiment, administering such treatment to a plurality of human patients results in an increase in the rate of OS compared to the rate of reference OS at 24 months of between 5% and 40% (e.g., between 5% and 20%, between 20% and 40%, between 10% and 30%, or between 15% and 25%; e.g., about 5%, 10%, 15%, 20%, 25%, 30%, 35%, or 40%).

In one embodiment, administering such treatment to a plurality of human patients results in an increase in the rate of OS compared to the rate of reference OS at 24 months of about 20%.

In one embodiment, the stratified hazard ratio is stratified by: (a) the number of previous lines of systemic therapy for DLBCL (1 vs. ≥2); and/or (b) the outcome of last systemic therapy (relapsed vs. refractory).

In one aspect, the invention features a method for treating relapsed or refractory diffuse large B-cell lymphoma (DLBCL) in a human patient in need thereof, comprising administering to the human patient an effective amount of: (a) glofitamab, (b) gemcitabine, and (c) oxaliplatin, wherein administering such treatment to a plurality of human patients results in a median duration of complete remission in the plurality of human patients of at least 27 months.

In one aspect, the invention features a method for treating relapsed or refractory diffuse large B-cell lymphoma (DLBCL) in a human patient in need thereof, comprising administering to the human patient an effective amount of: (a) glofitamab, (b) gemcitabine, and (c) oxaliplatin, wherein administering such treatment to the patient results in a complete remission in the patient, and wherein a plurality of human patients having received such treatment and exhibiting complete remission after end of treatment exhibits a rate of overall survival at 12 months of about 89%.

In one aspect, the invention features a method for treating relapsed or refractory diffuse large B-cell lymphoma (DLBCL) in a human patient in need thereof, comprising administering to the human patient an effective amount of: (a) glofitamab, (b) gemcitabine, and (c) oxaliplatin, wherein administering such treatment to the patient results in a complete remission in the patient, and wherein a plurality of human patients having received such treatment and exhibiting complete remission after end of treatment exhibits a rate of progression-free survival at 12 months of about 82%.

In one embodiment, the method comprises a first and a second dosing cycle, wherein:
the first dosing cycle comprises a first dose (C1D1) of about 2.5 mg of the glofitamab and a second dose (C1D2) of about 10 mg the glofitamab, and
the second dosing cycle comprises a single dose (C2D1) of about 30 mg of the glofitamab.

In one embodiment, the C1D1 and the C1D2 of the glofitamab are administered to the patient on Days 8 and 15, respectively, of the first dosing cycle.

In one embodiment, the C2D1 of the glofitamab is administered to the patient on Day 1 of the second dosing cycle.

In one embodiment, the first dosing cycle comprises a dose of 1000 mg obinutuzumab.

Ine one embodiment, the obinutuzumab is administered about 7 days before the first glofitamab dose.

In one embodiment, the obinutuzumab is administered on Day 1 of the first dosing cycle.

In one embodiment, the first and the second dosing cycle comprise a dose of 1000 mg/m² gemcitabine and a dose of 100 mg/m² oxaliplatin.

In one embodiment, gemcitabine and oxaliplatin are administered on Day 2 of the first dosing cycle.

In one embodiment, gemcitabine and oxaliplatin are administered on Day 1 or 2 of the second dosing cycle.

In one embodiment, gemcitabine is administered before oxaliplatin on the same day.

In one embodiment, the first and second dosing cycles are each 21-day dosing cycles.

In one embodiment the method comprises twelve dosing cycles.

In one embodiment, the dosing cycles are each 21-day dosing cycles.

In one embodiment the method comprises:
dosing cycle 1, wherein the patient is administered an effective dose of obinutuzumab, glofitamab, gemcitabine, and oxaliplatin,
dosing cycles 2 to 8, wherein the patient is administered an effective dose of glofitamab, gemcitabine, and oxaliplatin; and
dosing cycles 9 to 12, wherein the patient is administered an effective dose of glofitamab

In one embodiment, obinutuzumab is administered at a dose of 1000 mg on Day 1 of dosing cycle 1; glofitamab is administered at a dose of 0.5 mg on Day 8 and 10 mg on Day 15 of dosing cycle 1; and at a dose of 30 mg on Day 1 of dosing cycles 2 to 12; and gemcitabine is administered at a dose of 1000 mg/m² and oxaliplatin is administered at a dose of 100 mg/m² on Day 2 of dosing cycle 1 and on Day 1 or 2 of dosing cycles 2 to 8.

In one embodiment, gemcitabine is administered before oxaliplatin on the same day.

In one embodiment, glofitamab is provided for use in a method of treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
in the absence of glofitamab

In one embodiment, the invention relates to Glofitamab for use in a method of treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS,
   wherein the reference OS is the OS of a plurality of human patients who have received a control treatment comprising:
(a) rituximab,
(b) gemcitabine, and
(c) oxaliplatin,
in the absence of glofitamab.

In one embodiment, the invention relates to use of glofitamab in the manufacture of a medicament for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
in the absence of glofitamab.

In one embodiment, the invention relates to use of glofitamab in the manufacture of a medicament for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
in the absence of glofitamab.

In one aspect, the invention features a method of treating a patient having a CD20-positive cell proliferative disorder, e.g., a B cell proliferative disorder (e.g., an NHL (e.g., a relapsed and/or refractory NHL, a DLBCL (e.g., a relapsed and/or refractory DLBCL), a FL (e.g., a relapsed and/or refractory FL or a transformed FL), or an MCL (e.g., a relapsed or refractory MCL)), or a CNSL), comprising administering to the patient an effective amount of:
(a) glofitamab;
(b) gemcitabine; and
(c) oxaliplatin
   in a dosing regimen comprising at least a first dosing cycle and a second dosing cycle.

In one embodiment, the human patient has a relapsed or refractory DLBCL not otherwise specified (DLBCL NOS). In one embodiment, the human patient is not a candidate for hematopoietic stem cell transplantation (HSCT) (e.g., ineligible for HSCT). In one embodiment, the human patient has a relapsed or refractory DLBCL NOS and is not a candidate for HSCT (e.g., ineligible for HSCT).

In one embodiment, the first dosing cycle comprises a first dose (C1D1) of the glofitamab and a second dose (C1D2) of glofitamab, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of glofitamab, wherein the C2D1 of glofitamab is about 10 mg (e.g., 10 mg±0.05 mg, ±0.1 mg, ±0.2 mg, ±0.3 mg, ±0.5 mg, ±0.75 mg, or ±1 mg), about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg), or about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In particular embodiments, the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg) and the C1D2 of the glofitamab is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 is about 10 mg (e.g., 10 mg±0.05 mg, ±0.1 mg, ±0.2 mg, ±0.3 mg, ±0.5 mg, ±0.75 mg, or ±1 mg). In particular embodiments, the C2D1 of the glofitamab is about 16 mg (e.g., 16 mg±0.05 mg, ±0.1 mg, ±0.2 mg, ±0.3 mg, ±0.5 mg, ±0.75 mg, ±1 mg, ±1.5 mg, or ±1.6 mg). In particular embodiments, the C2D1 of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dosing cycle comprises a first dose (C1D1) of glofitamab and a second dose (C1D2) of glofitamab, wherein the C1D1 of glofitamab is about 2.5 mg (e.g., 2.5 mg±0.01 mg, ±0.02 mg, ±0.03 mg, ±0.05 mg, ±0.1 mg, ±0.2 mg, or ±0.25 mg), and the C1D2 of glofitamab is about 10 mg (e.g., 10 mg±0.05 mg, ±0.1 mg, ±0.2 mg, ±0.3 mg, ±0.5 mg, ±0.75 mg, or ±1 mg); and the second dosing cycle comprises a single dose (C2D1) of glofitamab, wherein the C2D1 of the glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In one embodiment, the first dose (C1D1) of glofitamab and the second dose (C1D2) of the glofitamab are administered to the patient on or about Days 8 (±1 day) and 15 (±1 day), respectively, of the first dosing cycle.

In one embodiment, the first dose (C1D1) of glofitamab and the second dose (C1D2) of the glofitamab are administered to the patient on Days 8 and 15, respectively, of the first dosing cycle. In some embodiments, the C2D1 of glofitamab is administered to the patient on or about Day 8 (±1 day) of the second dosing cycle. In some embodiments, the C2D1 of glofitamab is administered to the patient on or about Day 8 of the second dosing cycle.

In one embodiment, a single dose C1D1 of obinutuzumab is administered on Day 1 of the first cycle. In one embodiment, the single dose is about 1000 mg (e.g., 1000 mg±5 mg, ±10 mg, ±20 mg, ±30 mg, ±50 mg, ±75 mg, or ±100 mg).

In some embodiments, the methods featured by the invention further comprises administering to the patient one or more additional therapeutic agents. In some embodiments, the one or more additional therapeutic agents is tocilizumab. In one embodiment, the weight of the patient is greater than or equal to about 30 kg, and tocilizumab is administered at a dose of about 8 mg/kg (e.g., 8 mg/kg±0.05 mg/kg, ±0.1 mg/kg, ±0.25 mg/kg, ±0.5 mg/kg, or ±0.8 mg/kg). In one embodiment, the weight of the patient is less than 30 kg, and tocilizumab is administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg). In some embodiments, the maximum dose of tocilizumab is about 800 mg (e.g., 800 mg±10 mg, ±25 mg, ±50 mg, or ±80 mg).

In some embodiments, the one or more additional therapeutic agents is a corticosteroid. In some embodiments, the corticosteroid comprises prednisone, prednisolone, methylprednisolone, or dexamethasone. In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of glofitamab. In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab. In one embodiment, wherein methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of glofitamab. In one embodiment, methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of obinutuzumab. In one embodiment, prednisone is administered orally at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the glofitamab. In one embodiment, prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of glofitamab.

In some embodiments, one or more additional therapeutic agents is an antihistamine. In some embodiments, the antihistamine is diphenhydramine. In one embodiment, diphenhydramine is administered orally or intravenously at a dose of about 50 mg (e.g., 50 mg±0.5 mg, ±1 mg, ±1.5 mg, ±2 mg, ±3 mg, ±4 mg, or ±5 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of glofitamab.

In some embodiments, the one or more additional therapeutic agents comprises allopurinol and rasburicase.

In some embodiments, the one or more additional therapeutic agents is an antipyretic. In one embodiment, the antipyretic is acetaminophen or paracetamol. In one embodiment, acetaminophen or paracetamol is administered orally at a dose of between about 500 mg to about 1000 mg (e.g., 1000 mg±5 mg, ±10 mg, ±20 mg, ±30 mg, ±50 mg, ±75 mg, or ±100 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of glofitamab. In one embodiment, acetaminophen or paracetamol is administered orally at a dose of between about 500 mg to about 1000 mg (e.g., 1000 mg±5 mg, ±10 mg, ±20 mg, ±30 mg, ±50 mg, ±75 mg, or ±100 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of obinutuzumab.

In some embodiments, the one or more additional therapeutic agents comprises granulocyte colony-stimulating factor (G-CSF). In one embodiment, G-CSF is administered between about one day and about two days (e.g., 24, 26, 28, 30, 32, 36, 38, 40, 42, 44, 46, or 48 hours) after administration of any dose of rituximab, ifosfamide, carboplatin, and/or etoposide.

In some embodiments, the one or more additional therapeutic agents is mesna. In one embodiment, mesna is administered at a dose of about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), or about 166.6 mg/m² (e.g., 1666 mg/m²±25 mg/m², ±50 mg/m², ±100 mg/m², or ±166.6 mg/m²) intravenously.

Glofitamab is an anti-CD20/anti-CD3 bispecific antibody (WHO Drug Information (International Nonproprietary Names for Pharmaceutical Substances), Recommended INN: List 83, 2020, vol. 34, no. 1, p. 39 (incorporated herein by reference in its entirety); Proposed INN: List 121 WHO Drug Information, Vol. 33, No. 2, 2019, page 276 (incorporated herein by reference in its entirety), also known as CD20-TCB, RO7082859, or RG6026; CAS #: 2229047-91-8).

In one embodiment, glofitamab comprises two Fab molecules which specifically bind to CD20 comprising the following six hypervariable regions (HVRs):
(i) an HVR-H1 comprising the amino acid sequence of YSWIN (SEQ ID NO: 1);
(ii) an HVR-H2 comprising the amino acid sequence of RIFPGDGDTDYNGKFKG (SEQ ID NO: 2);
(iii) an HVR-H3 comprising the amino acid sequence of NVFDGYWLVY (SEQ ID NO:3);
(iv) an HVR-L1 comprising the amino acid sequence of RSSKSLLHSNGITYLY (SEQ ID NO: 4);
(v) an HVR-L2 comprising the amino acid sequence of QMSNLVS (SEQ ID NO: 5); and
(vi) an HVR-L3 comprising the amino acid sequence of AQNLELPYT (SEQ ID NO: 6).

In one embodiment, glofitamab comprises two Fab molecules which specifically bind to CD20 comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b).

In one embodiment, the Fab molecule which specifically binds to CD20 comprises (a) a VH domain comprising an amino acid sequence of SEQ ID NO: 7 and (b) a VL domain comprising an amino acid sequence of SEQ ID NO: 8.

In one embodiment, glofitamab comprises one Fab molecule which specifically binds to CD3 comprising the following six HVRs:
(i) an HVR-H1 comprising the amino acid sequence of TYAMN (SEQ ID NO: 9);
(ii) an HVR-H2 comprising the amino acid sequence of RIRSKYNNYATYYADSVKG (SEQ ID NO: 10);
(iii) an HVR-H3 comprising the amino acid sequence of HGNFGNSYVSWFAY (SEQ ID NO: 11);
(iv) an HVR-L1 comprising the amino acid sequence of GSSTGAVTTSNYAN (SEQ ID NO: 12);
(v) an HVR-L2 comprising the amino acid sequence of GTNKRAP (SEQ ID NO: 13); and
(vi) an HVR-L3 comprising the amino acid sequence of ALWYSNLWV (SEQ ID NO: 14).

In one embodiment, Glofitamab comprises one Fab molecule which specifically binds to CD3 comprising (a) a heavy chain variable (VH) domain comprising an amino acid sequence having at least 95% sequence identity to the amino acid sequence of SEQ ID NO: 15; (b) a light chain variable (VL) domain comprising an amino acid sequence having at least 95% sequence identity to the amino acid sequence of SEQ ID NO: 16; or (c) a VH domain as in (a) and a VL domain as in (b).

In one embodiment, the Fab molecule which specifically binds to CD3 comprises (a) a VH domain comprising an amino acid sequence of SEQ ID NO: 15 and (b) a VL domain comprising an amino acid sequence of SEQ ID NO: 16.

In a particular embodiment, glofitamab comprises a polypeptide that is at least 95%, 96%, 97%, 98%, or 99% identical to the sequence of SEQ ID NO: 17, a polypeptide that is at least 95%, 96%, 97%, 98%, or 99% identical to the sequence of SEQ ID NO: 18, a polypeptide that is at least 95%, 96%, 97%, 98%, or 99% identical to the sequence of SEQ ID NO: 19, and a polypeptide that is at least 95%, 96%, 97%, 98%, or 99% identical to the sequence of SEQ ID NO: 20. In a further particular embodiment, glofitamab comprises a polypeptide sequence of SEQ ID NO: 17, a polypeptide sequence of SEQ ID NO: 18, a polypeptide sequence of SEQ ID NO: 19 and a polypeptide sequence of SEQ ID NO: 20. In a further particular embodiment, glofitamab comprises one polypeptide chain comprising the amino acid sequence of SEQ ID NO: 17, one polypeptide chain comprising the amino acid sequence of SEQ ID NO: 18, one polypeptide chain comprising the amino acid sequence of SEQ ID NO: 19, and two polypeptide chains each comprising the amino acid sequence of SEQ ID NO: 20.

In a particular embodiment, glofitamab comprises a polypeptide that is at least 95%, 96%, 97%, 98%, or 99% identical to the sequence of SEQ ID NO: 58, a polypeptide that is at least 95%, 96%, 97%, 98%, or 99% identical to the sequence of SEQ ID NO: 59, a polypeptide that is at least 95%, 96%, 97%, 98%, or 99% identical to the sequence of SEQ ID NO: 19, and a polypeptide that is at least 95%, 96%, 97%, 98%, or 99% identical to the sequence of SEQ ID NO: 20. In a further particular embodiment, glofitamab comprises a polypeptide sequence of SEQ ID NO: 58, a polypeptide sequence of SEQ ID NO: 59, a polypeptide sequence of SEQ ID NO: 19 and a polypeptide sequence of SEQ ID NO: 20. In a further particular embodiment, glofitamab comprises one polypeptide chain comprising the amino acid sequence of SEQ ID NO: 58, one polypeptide chain comprising the amino acid sequence of SEQ ID NO: 59, one polypeptide chain comprising the amino acid sequence of SEQ ID NO: 19, and two polypeptide chains each comprising the amino acid sequence of SEQ ID NO: 20.

In some embodiments, the methods featured by the invention further comprises administering to the patient one or more additional therapeutic agents. In some embodiments, the one or more additional therapeutic agents is tocilizumab. In some embodiments, the one or more additional therapeutic agents is a corticosteroid. In some embodiments, the corticosteroid comprises prednisone, prednisolone, methylprednisolone, or dexamethasone. In some embodiments, one or more additional therapeutic agents is an antihistamine. In some embodiments, the antihistamine is diphenhydramine. In some embodiments, the one or more additional therapeutic agents comprises allopurinol and rasburicase. In some embodiments, the one or more additional therapeutic agents is an antipyretic. In some embodiments, the one or more additional therapeutic agents comprises granulocyte colony-stimulating factor (G-CSF). In some embodiments, the one or more additional therapeutic agents is mesna.

### (iv) CRS Risk Mitigation Strategies

The present invention relates to a new combination treatment of glofitamab and GemOx.

The methods and uses describe herein provide acceptable safety profiles for cytokine release syndrome in a population of patients having diffuse large B cell lymphoma (DLBCL), e.g., DLBCL not otherwise specified (DLBCL NOS) who are administered glofitamab, gemcitabine, and oxaliplatin (Glofit-GemOx). In some embodiments, the patients of the population are not candidates for hematopoietic stem cell transplantation (HSCT) or autologous stem cell transplantation (ASCT) (e.g., ineligible for HSCT or ASCT).

In some embodiments, population of patients exhibits a rate of cytokine release syndrome that is less than or equal to about 50% (e.g., less than or equal to about 40%, less than or equal to about 30%, less than or equal to about 20%, less than or equal to about 10%, less than or equal to about 5%, or less than or equal to about 1%; e.g., between about 0% to about 50%, between about 5% to about 40%, between about 5% to about 20%, between about 5% to about 10%, between about 20% to 20 about 50%, between about 30% to about 40%, between about 20% to about 40%, between about 15% to about 35%, between about 15% to about 25%, between about 35% to about 50%, or between about 25% to about 50%; e.g., about 50%, about 45%, about 40%, about 35%, about 30%, about 25%, about 20%, about 15%, about 10%, about 5%, about 1%, or about 0%).

In some embodiments, the rate of cytokine release syndrome of Grade ≥3 in the population of 25 patients is less than or equal to about 50% (e.g., less than or equal to about 40%, less than or equal to about 30%, less than or equal to about 20%, less than or equal to about 10%, less than or equal to about 5%, or less than or equal to about 1%; e.g., between about 0% to about 50%, between about 5% to about 40%, between about 5% to about 20%, between about 5% to about 10%, between about 20% to about 50%, between about 30% to about 40%, between about 20% to about 40%, between about 15% to about 30 35%, between about 15% to about 25%, between about 35% to about 50%, or between about 25% to about 50%; e.g., about 50%, about 45%, about 40%, about 35%, about 30%, about 25%, about 20%, about 15%, about 10%, about 5%, about 1%, or about 0%). In some embodiments, the rate of cytokine release syndrome of Grade ≥3 in the population of patients is less than or equal to about 20%. In particular embodiments, the rate of cytokine release syndrome of Grade ≥3 in the population of patients 35 is less than or equal to about 10%. In particular embodiments, the rate of cytokine release syndrome of Grade ≥3 in the population of patients is less than or equal to about 5%.

Glofitamab involved T-cell activation have been associated with cytokine release syndrome (CRS). CRS is a potentially life-threatening symptom complex caused by the excessive release of cytokines by immune effector or target cells during an exaggerated and sustained immune response. CRS can be triggered by a variety of factors, including infection with virulent pathogens, or by medications that activate or enhance the immune response, resulting in a pronounced and sustained immune response.

Regardless of the inciting agent, severe or life-threatening CRS is a medical emergency. If unsuccessfully managed, it can result in significant disability or fatal outcome. Current clinical management focuses on treating the individual signs and symptoms, providing supportive care, and attempting to dampen down the inflammatory response using high-dose corticosteroids. However, this approach is not always successful, especially in the case of late intervention. Moreover, steroids may negatively impact T-cell function, which may diminish the clinical benefit of immune modulating therapies in the treatment of cancer.

### A. CRS Symptoms and Grading

CRS is graded according to the Modified Cytokine Release Syndrome Grading System established by Lee et al., Blood, 124:188-195, 2014 or Lee et al., Biol Blood Marrow Transplant, 25 (4): 625-638, 2019, as described in Table 3. In addition to diagnostic criteria, recommendations on management of CRS based on its severity, including early intervention with corticosteroids and/or anti-cytokine therapy, are provided and referenced in Tables 3 and 4.

TABLE 3
Cytokine release syndrome grading systems

| | Modified Cytokine Release | ASTCT Consensus Grading |
|---|---|---|
| Grade | Syndrome Grading System | System |
| Grade 1 | Symptoms are not life threatening | Temperature ≥38° C. |
| | and require symptomatic treatment | No hypotension |
| | only (e.g., fever, nausea, fatigue, | No hypoxia |
| | headache, myalgia, malaise) | |
| Grade 2 | Symptoms require and respond to | Temperature ≥38° C.* with |
| | moderate intervention | hypotension not requiring |
| | Oxygen requirement <40%; or | vasopressors and/or^{†} hypoxia |
| | Hypotension responsive to fluids or | requiring low-flow nasal |
| | low dose^{a} of one vasopressor; or | cannula^{‡} or blow-by |
| | Grade 2 organ toxicity | |
| Grade 3 | Symptoms require and respond to | Temperature ≥38° C.* with |
| | aggressive intervention | hypotension requiring a |
| | Oxygen requirement ≥40%; or | vasopressor with or without |
| | Hypotension requiring high dose^{b} | vasopressin and/or^{†} hypoxia |
| | or multiple vasopressors; or | requiring high-flow nasal |
| | Grade 3 organ toxicity or Grade 4 | cannula^{‡}, facemask, |
| | transaminitis | nonrebreather mask, or |
| | | Venturi mask |
| Grade 4 | Life-threatening symptoms | Temperature ≥38° C.* with |
| | Requirement for ventilation support | hypotension requiring |
| | or | multiple vasopressors |
| | Grade 4 organ toxicity (excluding | (excluding vasopressin) |
| | transaminitis) | and/or^{†} hypoxia |
| | | requiring positive pressure |
| | | (e.g., CPAP, BiPAP, |
| | | intubation and mechanical |
| | | ventilation) |
| Grade 5^{c} | Death | Death |

Lee 2014 criteria: Lee et al., Blood, 124: 188-195, 2014.
ASTCT consensus grading: Lee et al., Biol Blood Marrow Transplant, 25(4): 625-638, 2019.
BiPAP = bilevel positive airway pressure;
C-PAP = continuous positive airway pressure;
CRS = cytokine release syndrome;
ASTCT = American Society for Transplantation and Cellular Therapy
NCI CTCAE v5.0 = National Cancer Institute Common Terminology Criteria for Adverse Events, Version 5.0.
Note:
Organ toxicities associated with CRS may be graded according to NCI CTCAE v5.0 but they do not influence CRS grading.
^{a}Low-dose vasopressor: single vasopressor at doses below that shown in Table 4.
^{b}High-dose vasopressor: as defined in Table 4.
^{c}Grade 5 CRS is defined as death due to CRS.
*Fever is defined as temperature ≥38° C. not attributable to any other cause. In patients who have CRS then receive antipyretic or anticytokine therapy such as tocilizumab or steroids, fever is no longer required to grade subsequent CRS severity. In this case, CRS grading is driven by hypotension and/or hypoxia.
^{†}CRS grade is determined by the more severe event: hypotension or hypoxia not attributable to any other cause. For example, a patient with temperature of 39.5° C., hypotension requiring one vasopressor, and hypoxia requiring low-flow nasal cannula is classified as grade 3 CRS.
^{‡}Low-flow nasal cannula is defined as oxygen delivered at ≤6L/minute. Low flow also includes blow-by oxygen delivery, sometimes used in pediatrics. High-flow nasal cannula is defined as oxygen delivered at >6 L/minute.

TABLE 4
High-dose vasopressors
High-Dose Vasopressors (duration ≥3 hours)

| Pressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine equivalent of ≥10 μg/min ^{a} |
| If on combination or vasopressors | Norepinephrine equivalent of ≥20 μg/min ^{a} |
| (not vasopressin) | |

min = minute; VASST = Vasopressin and Septic Shock Trial.
^{a} VASST vasopressor equivalent equation: norepinephrine equivalent dose = [norepinephrine (μg/min)] + [dopamine (μg/kg/min) ÷ 2] + [epinephrine (μg/min)] + [phenylephrine (μg/min) ÷ 10].

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS). Standard of care for severe or life-threatening CRS resulting from immune-based therapy has not been established; case reports and recommendations using anti-cytokine therapy such as tocilizumab have been published (Teachey et al., Blood, 121:5154-5157, 2013; Lee et al., Blood, 124:188-195, 2014; Maude et al., New Engl J Med, 371:1507-1517, 2014).

B. Optimized Regimen of Steroids for the Treatment of Patients with Glofitamab

Glofitamab can induce cytokine release syndrome (CRS), a significant side effect that requires management. Results from preclinical in vivo studies indicate that dexamethasone is a viable option for managing CRS in patients treated with glofitamab, enhancing the safety profile of the therapy without diminishing its antitumor potential. Provided herein is an optimized regimen of steroids for the treatment of patients with glofitamab to optimize the CRS profile of this treatment.

The invention provides methods for treating a patient having a CD20-positive cell proliferative disorder (e.g., a B cell proliferative disorder (e.g., non-Hodgkin's lymphoma (NHL) (e.g., a relapsed and/or refractory NHL, a diffuse-large B cell lymphoma (DLBCL) (e.g., a relapsed and/or refractory DLBCL), a follicular lymphoma (FL) (e.g., a relapsed and/or refractory FL or a transformed FL), or a mantle cell lymphoma (MCL) (e.g., a relapsed or refractory MCL)), or a central nervous system lymphoma (CNSL))) that includes administering to the patient glofitamab in combination with GemOx as described herein, wherein patient receives corticosteroid prophylaxis prior to and after administration of glofitamab.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein patient receives prophylaxis prior to and after administration of glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
in the absence of glofitamab, wherein patient receives corticosteroid prophylaxis prior to and after administration of glofitamab.

In one embodiment, the corticosteroid prophylaxis comprises prednisolone and methylprednisolone, and/or dexamethasone. In one embodiment, the corticosteroid prophylaxis comprises dexamethasone. In one embodiment, the corticosteroid prophylaxis comprises 20 mg dexamethasone.

In one embodiment, the corticosteroid prophylaxis is administered one day prior to administration of glofitamab. In one embodiment, the corticosteroid prophylaxis is administered about 24 hours prior to administration of glofitamab.

In one embodiment, the corticosteroid prophylaxis is administered about 30 hours, about 29 hours, about 28 hours, about 27 hours, about 26 hours, about 25 hours, about 24 hours, about 23 hours, about 22 hours, about 21 hours, about 20 hours, about 19 hours, about 18 hours prior to administration of glofitamab.

In one embodiment, the corticosteroid prophylaxis is administered on the same day as administration of glofitamab.

In one embodiment, the corticosteroid prophylaxis is administered about 30-90 or about 60 minutes prior to the glofitamab administration. In one embodiment the corticosteroid prophylaxis is administered about 25, about 30, about 40, about 50, about 60, about 70, about 80, about 90, or about 100 minutes prior to administration of glofitamab.

In one embodiment, the corticosteroid prophylaxis is administered one day after administration of glofitamab.

In one embodiment, the corticosteroid prophylaxis is administered about 24 hours after administration of glofitamab.

In one embodiment, the corticosteroid prophylaxis is administered about 30 hours, about 29 hours, about 28 hours, about 27 hours, about 26 hours, about 25 hours, about 24 hours, about 23 hours, about 22 hours, about 21 hours, about 20 hours, about 19 hours, about 18 hours after administration of glofitamab.

In one embodiment, the corticosteroid prophylaxis is administered before the first dose (C1D1) of glofitamab. In one embodiment, the corticosteroid prophylaxis is administered before the second dose (C1D2) of glofitamab. In one embodiment, the corticosteroid prophylaxis is administered one day prior to the glofitamab administration, prior to the glofitamab administration on the same day, and one day after the glofitamab administration.

In one embodiment, the corticosteroid prophylaxis is administered about 24 hours prior to the glofitamab administration, about 30-90 or about 60 minutes prior to the glofitamab administration and about 24 hours after the glofitamab administration.

In one embodiment, the corticosteroid prophylaxis is dexamethasone. In one embodiment, dexamethasone is administered at a dose of 20 mg.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, wherein the corticosteroid prophylaxis is administered before the third dose (C2D1) of glofitamab.

In one embodiment, the corticosteroid prophylaxis is administered before any subsequent dose of glofitamab if the patient has experienced CRS with any previous 30 mg dose of glofitamab.

In one embodiment, the corticosteroid prophylaxis comprises 20 mg dexamethasone.

In one embodiment, the incidence of CRS in a plurality of patients is reduced compared to treatment wherein corticosteroid prophylaxis consists of one dose of a corticosteroid on the same day as Glofitamab administration.

In one embodiment, the patient does not have a Grade 3 CRS event.

In one embodiment, the patient does not need to be hospitalized after treatment with glofitamab.

In one embodiment, the rate of the cytokine release syndrome of a Grade of 2 or greater (as defined by the American Society for Transplantation and Cellular Therapy, 2019; ASTCT) is less than about 10%, less than about 5%, or less than about 2%.

In one embodiment the cycles are 21 days in length.

In one embodiment, the premedication with a corticosteroid results in superior anti-tumor activity, compared to patients treated without the corticosteroid premedication.

In one embodiment, in the event of a late or prolonged infusion for glofitamab during dosing cycles 2-12 because of a late start in the day or slowed infusion rate due to an IRR or CRS, GemOx is administered on Day 2 of the respective cycle.

In one embodiment, there is an observation period between the end of infusion of glofitamab and before GemOx infusion. In one embodiment said observation period is about 90 minutes long. In one embodiment said observation period is 90 minutes±15 minutes long. At Cycle 3 and beyond, if the participant has not had CRS with prior cycles and has tolerated the preceding glofitamab infusion with no signs or symptoms of CRS, the window of observation is less than about 90 minutes. In one embodiment said observation period is 60 minutes±20 minutes, 60 minutes±15 minutes, or 60 minutes±10 minutes long.

In one embodiment, a first dose (C1D1) of 2.5 mg of glofitamab on Day 8 of the first dosing cycle is infused over 4 hours (±15 minutes), followed by 4 hours (±15 minutes), of monitoring prior to discharge, In one embodiment, the second dose (C1D2) of 10 mg glofitamab on Day 15 of the first dosing cycle is infused over 4 hours (±15 minutes), followed by 4 hours (±15 minutes), of monitoring prior to discharge. In one embodiment, the dose of 30 mg glofitamab on Day 1 of the second dosing cycle is infused over 4 hours (±15 minutes), followed by 90 minutes (±30 minutes), of monitoring prior to discharge. In one embodiment, the dose of 30 mg glofitamab on Day 1 of dosing cycles 2-12 is infused over 4 hours (±15 minutes), followed by 90 minutes (±30 minutes), of monitoring prior to discharge.

In one embodiment, the dose of 30 mg on Day 1 of dosing cycle 3-12 is infused over 2 hours (±15 minutes) and the window of observation is less than 90 minutes.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the patient receives dexamethasone one day prior to administration of glofitamab, on the same day as glofitamab, and one day after glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
   (2) the second dosing cycle comprises a single dose (C2D1) of 30 mg of glofitamab, and wherein the patient receives dexamethasone one day prior to administration of glofitamab, on the same day as glofitamab, and one day after glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
   (2) the second dosing cycle comprises a single dose (C2D1) of 30 mg of glofitamab, and wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin, and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab and one day after glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin, and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and
   wherein the patient receives dexamethasone one day prior to administration of glofitamab, on the same day as glofitamab and one day after glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and
   wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab, gemcitabine, oxaliplatin, and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle, and
   (2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one embodiment, a dose of 1000 mg/m² of the gemcitabine and a dose of 100 mg/m² of oxaliplatin is administered on Day 1 of subsequent dosing cycles.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle
   (2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

. In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one aspect, the invention features a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of the second dosing cycle and on Day 1 of dosing cycles 3 to 8; and
(3) dosing cycles 9 to 12, wherein the patient is administered a single dose of 30 mg of glofitamab on Day 1.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of the second dosing cycle and on Day 1 of dosing cycles 3 to 8; and
(3) dosing cycles 9 to 12, wherein the patient is administered a single dose of 30 mg of glofitamab on Day 1.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method
comprising administering to the human patient an effective amount of:
   (a) glofitamab,
   (b) gemcitabine, and
   (c) oxaliplatin,
wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
   (a) rituximab,
   (b) gemcitabine, and
   (c) oxaliplatin,
      in the absence of glofitamab, wherein patient receives corticosteroid prophylaxis prior to and after administration of glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
in the absence of glofitamab, wherein patient receives corticosteroid prophylaxis prior to and after administration of glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the patient receives dexamethasone one day prior to administration of glofitamab, on the same day as glofitamab and one day after glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
   (2) the second dosing cycle comprises a single dose (C2D1) of 30 mg of glofitamab, and wherein the patient receives dexamethasone one day prior to administration of glofitamab, on the same day as glofitamab and one day after glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
   (2) the second dosing cycle comprises a single dose (C2D1) of 30 mg of glofitamab, and wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin, and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab and one day after glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin, and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and wherein the patient receives dexamethasone one day prior to administration of glofitamab, on the same day as glofitamab and one day after glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and
   wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab, gemcitabine, oxaliplatin, and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle; and
   (2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab, gemcitabine, oxaliplatin, and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle; and
   (2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of the second dosing cycle and on Day 1 of dosing cycles 3 to 8; and
(3) dosing cycles 9 to 12, wherein the patient is administered a single dose of 30 mg of glofitamab on Day 1.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS,
   wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of the second dosing cycle and on Day 1 of dosing cycles 3 to 8; and
(3) dosing cycles 9 to 12, wherein the patient is administered a single dose of 30 mg of glofitamab on Day 1.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method
comprising administering to the human patient an effective amount of:
   (a) glofitamab,
   (b) gemcitabine, and
   (c) oxaliplatin, wherein patient receives prophylaxis prior to and after administration of glofitamab.

In one embodiment, the corticosteroid prophylaxis comprises prednisolone and methylprednisolone, and/or dexamethasone. In one embodiment, the corticosteroid prophylaxis comprises dexamethasone. In one embodiment, the corticosteroid prophylaxis comprises 20 mg dexamethasone.

In one embodiment, the corticosteroid prophylaxis is administered one day prior to administration of glofitamab. In one embodiment, the corticosteroid prophylaxis is administered about 24 hours prior to administration of glofitamab. In one embodiment, the corticosteroid prophylaxis is administered on the same day as administration of glofitamab.

In one embodiment, the corticosteroid prophylaxis is administered about 30-90 or about 60 minutes prior to the glofitamab administration. In one embodiment, the corticosteroid prophylaxis is administered one day after administration of glofitamab.

In one embodiment, the corticosteroid prophylaxis is administered about 24 hours after administration of glofitamab. In one embodiment, the corticosteroid prophylaxis is administered before the first dose (C1D1) of glofitamab. In one embodiment, the corticosteroid prophylaxis is administered before the second dose (C1D2) of glofitamab. In one embodiment, the corticosteroid prophylaxis is administered one day prior to the glofitamab administration, prior to the glofitamab administration on the same day, and one day after the glofitamab administration.

In one embodiment, the corticosteroid prophylaxis is administered about 24 hours prior to the glofitamab administration, about 30-90 or about 60 minutes prior to the glofitamab administration, and about 24 hours after the glofitamab administration.

In one embodiment, the corticosteroid prophylaxis is dexamethasone. In one embodiment, dexamethasone is administered at a dose of 20 mg.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, wherein the corticosteroid prophylaxis is administered before the third dose (C2D1) of glofitamab.

In one embodiment, the corticosteroid prophylaxis is administered before any subsequent dose of glofitamab if the patient has experienced CRS with any previous 30 mg dose of glofitamab.

In one embodiment, the corticosteroid prophylaxis comprises 20 mg dexamethasone.

In one embodiment, the incidence of CRS in a plurality of patients is reduced compared to treatment wherein corticosteroid prophylaxis consists of one dose of a corticosteroid on the same day as glofitamab administration.

In one embodiment, the patient does not have a Grade 3 CRS event.

In one embodiment, the patient does not need to be hospitalized after treatment with glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin, wherein the patient receives dexamethasone one day prior to administration of glofitamab, on the same day as glofitamab, and one day after glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
   (2) the second dosing cycle comprises a single dose (C2D1) of 30 mg of glofitamab, and wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin, and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and wherein the patient receives dexamethasone one day prior to administration of glofitamab, on the same day as glofitamab and one day after glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab, gemcitabine, oxaliplatin, and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab, gemcitabine, oxaliplatin, and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle; and
   (2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one embodiment, a dose of 1000 mg/m² of the gemcitabine and a dose of 100 mg/m² of oxaliplatin is administered on Day 1 of subsequent dosing cycles.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein the method comprises:
(1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:

(a) glofitamab,
(b) gemcitabine, and
(c) oxaliplatin,
   wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one aspect, the invention features a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of dosing cycles 2 to 8.
(3) dosing cycles 9 to 12, wherein the patient is administered a single dose of 30 mg of glofitamab on Day 1.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein patient receives corticosteroid prophylaxis prior to and after administration of glofitamab.

In one aspect, glofitamab for use in a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
in the absence of glofitamab, wherein patient receives corticosteroid prophylaxis prior to and after administration of glofitamab.

In one embodiment, the corticosteroid prophylaxis comprises prednisolone and methylprednisolone, and/or dexamethasone. In one embodiment, the corticosteroid prophylaxis comprises dexamethasone. In one embodiment, the corticosteroid prophylaxis comprises 20 mg dexamethasone.

In one embodiment, the corticosteroid prophylaxis is administered one day prior to administration of glofitamab. In one embodiment, the corticosteroid prophylaxis is administered about 24 hours prior to administration of glofitamab. In one embodiment, the corticosteroid prophylaxis is administered on the same day as administration of glofitamab.

In one embodiment, the corticosteroid prophylaxis is administered about 30-90 or about 60 minutes prior to the glofitamab administration. In one embodiment, the corticosteroid prophylaxis is administered one day after administration of glofitamab.

In one embodiment, the corticosteroid prophylaxis is administered about 24 hours after administration of glofitamab. In one embodiment, the corticosteroid prophylaxis is administered before the first dose (C1D1) of glofitamab. In one embodiment, the corticosteroid prophylaxis is administered before the second dose (C1D2) of glofitamab. In one embodiment, the corticosteroid prophylaxis is administered one day prior to the glofitamab administration, prior to the glofitamab administration on the same day, and one day after the glofitamab administration.

In one embodiment, the corticosteroid prophylaxis is administered about 24 hours prior to the glofitamab administration, about 30-90 or about 60 minutes prior to the glofitamab administration, and about 24 hours after the glofitamab administration.

In one embodiment, the corticosteroid prophylaxis is dexamethasone. In one embodiment, dexamethasone is administered at a dose of 20 mg.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the corticosteroid prophylaxis is administered before the third dose (C2D1) of glofitamab.

In one embodiment, the corticosteroid prophylaxis is administered before any subsequent dose of glofitamab if the patient has experienced CRS with any previous 30 mg dose of glofitamab.

In one embodiment, the corticosteroid prophylaxis comprises 20 mg dexamethasone.

In one embodiment, the incidence of CRS in a plurality of patients is reduced compared to treatment wherein corticosteroid prophylaxis consists of one dose of a corticosteroid on the same day as Glofitamab administration.

In one embodiment, the patient does not have a Grade 3 CRS event.

In one embodiment, the patient does not need to be hospitalized after treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the patient receives dexamethasone one day prior to administration of glofitamab, on the same day as glofitamab and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration, and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration, and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, glofitamab for use in a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the patient receives dexamethasone one day prior to administration of glofitamab, on the same day as glofitamab and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
   (2) the second dosing cycle comprises a single dose (C2D1) of 30 mg of glofitamab, and wherein the patient receives dexamethasone one day prior to administration of glofitamab, on the same day as glofitamab and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, glofitamab for use in a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
   (2) the second dosing cycle comprises a single dose (C2D1) of 30 mg of glofitamab, and wherein the patient receives dexamethasone one day prior to administration of glofitamab, on the same day as glofitamab and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of Glofitamab and a second dose (C1D2) of 10 mg of glofitamab and a dose of 1000 mg/m² of the gemcitabine and a dose of 100 mg/m² of the oxaliplatin; and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin, and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and
   wherein the patient receives dexamethasone one day prior to administration of glofitamab, on the same day as glofitamab and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS,
   wherein the reference OS is the OS of a plurality of human patients who have received a control treatment comprising:
(a) rituximab,
(b) gemcitabine, and
(c) oxaliplatin,
   in the absence of glofitamab, wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and wherein the patient receives dexamethasone one day prior to administration of glofitamab, on the same day as glofitamab and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab, gemcitabine, oxaliplatin, and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade of 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle; and
   (2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one embodiment, a dose of 1000 mg/m² of the gemcitabine and a dose of 100 mg/m² of oxaliplatin is administered on Day 1 of subsequent dosing cycles.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for

Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle, and
   (2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.
   In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, a dose of 1000 mg/m² of the gemcitabine and a dose of 100 mg/m² of oxaliplatin is administered on Day 1 of subsequent dosing cycles.

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(4) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(5) dosing cycles 2 to 8, wherein the patient is administered a single dose of 30 mg of glofitamab and an effective dose of gemcitabine, and oxaliplatin; and
(6) dosing cycles 9 to 12, wherein the patient is administered a single dose of 30 mg of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of dosing cycles 2 to 8; and
(3) dosing cycles 9 to 12, wherein the patient is administered a single dose of 30 mg of glofitamab on Day 1.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of dosing cycles 2 to 8.
(3) dosing cycles 9 to 12, wherein the patient is administered a single dose of 30 mg of glofitamab on Day 1.

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method
comprising administering to the human patient an effective amount of:
   (a) glofitamab,
   (b) gemcitabine, and
   (c) oxaliplatin,
wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
   (a) rituximab,
   (b) gemcitabine, and
   (c) oxaliplatin,
      in the absence of glofitamab, wherein patient receives corticosteroid prophylaxis prior to and after administration of glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
in the absence of glofitamab, wherein patient receives corticosteroid prophylaxis prior to and after administration of glofitamab.

In one embodiment, the corticosteroid prophylaxis comprises prednisolone and methylprednisolone, and/or dexamethasone. In one embodiment, the corticosteroid prophylaxis comprises dexamethasone. In one embodiment, the corticosteroid prophylaxis comprises 20 mg dexamethasone.

In one embodiment, the corticosteroid prophylaxis is administered one day prior to administration of glofitamab. In one embodiment, the corticosteroid prophylaxis is administered about 24 hours prior to administration of glofitamab. In one embodiment, the corticosteroid prophylaxis is administered on the same day as administration of glofitamab.

In one embodiment, the corticosteroid prophylaxis is administered about 30-90 or about 60 minutes prior to the glofitamab administration. In one embodiment, the corticosteroid prophylaxis is administered one day after administration of glofitamab.

In one embodiment, the corticosteroid prophylaxis is administered about 24 hours after administration of glofitamab. In one embodiment, the corticosteroid prophylaxis is administered before the first dose (C1D1) of glofitamab. In one embodiment, the corticosteroid prophylaxis is administered before the second dose (C1D2) of glofitamab. In one embodiment, the corticosteroid prophylaxis is administered one day prior to the glofitamab administration, prior to the glofitamab administration on the same day, and one day after the glofitamab administration.

In one embodiment, the corticosteroid prophylaxis is administered about 24 hours prior to the glofitamab administration, about 30-90 or about 60 minutes prior to the glofitamab administration, and about 24 hours after the glofitamab administration.

In one embodiment, the corticosteroid prophylaxis is dexamethasone. In one embodiment, dexamethasone is administered at a dose of 20 mg.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, wherein the corticosteroid prophylaxis is administered before the third dose (C2D1) of glofitamab.

In one embodiment, the corticosteroid prophylaxis is administered before any subsequent dose of glofitamab if the patient has experienced CRS with any previous 30 mg dose of glofitamab.

In one embodiment, the corticosteroid prophylaxis comprises 20 mg dexamethasone.

In one embodiment, the incidence of CRS in a plurality of patients is reduced compared to treatment wherein corticosteroid prophylaxis consists of one dose of a corticosteroid on the same day as Glofitamab administration.

In one embodiment, the patient does not have a Grade 3 CRS event.

In one embodiment, the patient does not need to be hospitalized after treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the patient receives dexamethasone one day prior to administration of glofitamab, on the same day as glofitamab and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
(2) the second dosing cycle comprises a single dose (C2D1) of 30 mg of glofitamab, and wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
   (2) the second dosing cycle comprises a single dose (C2D1) of 30 mg of glofitamab, and wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features a glofitamab for use in method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin, and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin, and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and
   wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab, gemcitabine, oxaliplatin, and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab
   wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle, and
   (2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one embodiment, a dose of 1000 mg/m² of the gemcitabine and a dose of 100 mg/m² of oxaliplatin is administered on Day 1 of subsequent dosing cycles.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab,
   wherein glofitamab, gemcitabine, oxaliplatin and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle, and
   (2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, a dose of 1000 mg/m² of the gemcitabine and a dose of 100 mg/m² of oxaliplatin is administered on Day 1 of subsequent dosing cycles.

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of dosing cycles 2 to 8.
(3) dosing cycles 9 to 12, wherein the patient is administered a single dose of 30 mg of glofitamab on Day 1.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, a method for treating relapsed or refractory diffuse large B-cell lymphoma (DLBCL) in a human patient in need thereof as described herein is provided, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
   in the absence of glofitamab, wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of dosing cycles 2 to 8.
(3) dosing cycles 9 to 12, wherein the patient is administered a single dose of 30 mg of glofitamab on Day 1.

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method
comprising administering to the human patient an effective amount of:
   (a) glofitamab,
   (b) gemcitabine, and
   (c) oxaliplatin, wherein patient receives corticosteroid prophylaxis prior to and after administration of glofitamab.

In one embodiment, the corticosteroid prophylaxis comprises prednisolone and methylprednisolone, and/or dexamethasone. In one embodiment, the corticosteroid prophylaxis comprises dexamethasone. In one embodiment, the corticosteroid prophylaxis comprises 20 mg dexamethasone.

In one embodiment, the corticosteroid prophylaxis is administered one day prior to administration of glofitamab. In one embodiment, the corticosteroid prophylaxis is administered about 24 hours prior to administration of glofitamab. In one embodiment, the corticosteroid prophylaxis is administered on the same day as administration of glofitamab.

In one embodiment, the corticosteroid prophylaxis is administered about 30-90 or about 60 minutes prior to the glofitamab administration. In one embodiment, the corticosteroid prophylaxis is administered one day after administration of glofitamab.

In one embodiment, the corticosteroid prophylaxis is administered about 24 hours after administration of glofitamab. In one embodiment, the corticosteroid prophylaxis is administered before the first dose (C1D1) of glofitamab. In one embodiment, the corticosteroid prophylaxis is administered before the second dose (C1D2) of glofitamab. In one embodiment, the corticosteroid prophylaxis is administered one day prior to the glofitamab administration, prior to the glofitamab administration on the same day, and one day after the glofitamab administration.

In one embodiment, the corticosteroid prophylaxis is administered about 24 hours prior to the glofitamab administration, about 30-90 or about 60 minutes prior to the glofitamab administration, and about 24 hours after the glofitamab administration.

In one embodiment, the corticosteroid prophylaxis is dexamethasone. In one embodiment, dexamethasone is administered at a dose of 20 mg.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, wherein the corticosteroid prophylaxis is administered before the third dose (C2D1) of glofitamab.

In one embodiment, the corticosteroid prophylaxis is administered before any subsequent dose of glofitamab if the patient has experienced CRS with any previous 30 mg dose of glofitamab.

In one embodiment, the corticosteroid prophylaxis comprises 20 mg dexamethasone.

In one embodiment, the incidence of CRS in a plurality of patients is reduced compared to treatment wherein corticosteroid prophylaxis consists of one dose of a corticosteroid on the same day as Glofitamab administration.

In one embodiment, the patient does not have a Grade 3 CRS event.

In one embodiment, the patient does not need to be hospitalized after treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin, wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab; and
   (2) the second dosing cycle comprises a single dose (C2D1) of 30 mg of glofitamab, and wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab and dexamethasone are administered in a dosing regimen comprising at least a first dosing cycle and a second dosing cycle, wherein:
   (1) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin, and
   (2) the second dosing cycle comprises a single dose (C2D1) of 30 mg of glofitamab and a dose of 1000 mg/m² of the gemcitabine and a dose of 100 mg/m² of the oxaliplatin,
   and wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and
   (2) a single dose (C2D1) of 30 mg of glofitamab is administered on Day 1 of the second dosing cycle, and
   wherein the patient receives dexamethasone one day prior to administration of glofitamab, on the same day as glofitamab, and one day after glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab, gemcitabine, oxaliplatin, and dexamethasone are administered in a dosing regimen comprising at least a first dosing cycle and a second dosing cycle, wherein:
   (1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and
   (2) a single dose (C2D1) of 30 mg of glofitamab is administered on Day 1 of the second dosing cycle and wherein the first dosing cycle and the second dosing cycle comprises a dose of 1000 mg/m² of the gemcitabine and a dose of 100 mg/m² of the oxaliplatin.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein glofitamab, gemcitabine, oxaliplatin and dexamethasone are administered in a dosing regimen comprising at least a first dosing cycle and a second dosing cycle, wherein:
(1) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle, and
(2) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one embodiment, a dose of 1000 mg/m² of the gemcitabine and a dose of 100 mg/m² of oxaliplatin is administered on Day 1 of subsequent dosing cycles.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for

Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein the method comprises:
(1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features glofitamab for use in a method of treating relapsed or refractory DLBCL in a human patient in need thereof as described herein, wherein the patient has relapsed after or is refractory to one prior line of therapy and is ineligible for hematopoietic stem cell transplantation, the method comprising administering to the human patient an effective amount of:
(a) glofitamab,
(b) gemcitabine, and
(c) oxaliplatin,
   wherein the method comprises:
(1) dosing cycle 1, wherein the patient is administered an effective dose of the obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; wherein gemcitabine and oxaliplatin are administered on Day 1 of dosing cycles 2 to 8.
(3) dosing cycles 9 to 12, wherein the patient is administered a single dose of 30 mg of glofitamab on Day 1.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

### C. Method of Reducing the Incidence of CRS Events

In one aspect, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and
(b) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one aspect, the invention features a method of reducing the likelihood of a CRS event in a CD20-positive B cell proliferative disorder patient treated with glofitamab, comprising administering to the patient glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and
(b) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, dexamethasone is administered for the third dose (C2D1) of glofitamab if the patient has experienced CRS with first and or second dose of glofitamab.

In one embodiment, dexamethasone is administered before any subsequent dose of glofitamab if the patient has experienced CRS with any previous 30 mg dose of glofitamab.

In one embodiment, dexamethasone is administered for the first and second dose (C2D1) of glofitamab and not for any subsequent dose.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one embodiment, the rate of the cytokine release syndrome of a Grade of 2 or greater (as defined by the American Society for Transplantation and Cellular Therapy, 2019; ASTCT) is less than about 10%, less than about 5%, or less than about 2%.

In one embodiment the cycles are 21 days in length.

In one embodiment, the premedication with a corticosteroid results in superior anti-tumor activity, compared to patients treated without the corticosteroid premedication.

In one embodiment, in the event of a late or prolonged infusion for glofitamab during dosing cycles 2-12 because of a late start in the day or slowed infusion rate due to an IRR or CRS, GemOx is administered on Day 2 of the respective cycle.

In one embodiment, there is an observation period between the end of infusion of glofitamab and before GemOx infusion. In one embodiment said observation period is about 90 minutes long. In one embodiment said observation period is 90 minutes±15 minutes long. At Cycle 3 and beyond, if the participant has not had CRS with prior cycles and has tolerated the preceding glofitamab infusion with no signs or symptoms of CRS, the window of observation is less than about 90 minutes. In one embodiment said observation period is 60 minutes±20 minutes, 60 minutes±15 minutes, or 60 minutes±10 minutes long.

In one embodiment, a first dose (C1D1) of 2.5 mg of glofitamab on Day 8 of the first dosing cycle is infused over 4 hours (±15 minutes), followed by 4 hours (±15 minutes), of monitoring prior to discharge, In one embodiment, the second dose (C1D2) of 10 mg glofitamab on Day 15 of the first dosing cycle is infused over 4 hours (±15 minutes), followed by 4 hours (±15 minutes), of monitoring prior to discharge. In one embodiment, the dose of 30 mg glofitamab on Day 1 of the second dosing cycle is infused over 4 hours (±15 minutes), followed by 90 minutes (±30 minutes), of monitoring prior to discharge. In one embodiment, the dose of 30 mg glofitamab on Day 1 of dosing cycles 2-12 is infused over 4 hours (±15 minutes), followed by 90 minutes (±30 minutes), of monitoring prior to discharge.

In one embodiment, the dose of 30 mg on Day 1 of dosing cycle 3-12 is infused over 2 hours (±15 minutes) and the window of observation is less than 90 minutes.

In one embodiment, the patient has relapsed after or is refractory to one prior line of therapy and is ineligible for hematopoietic stem cell transplantation.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
glofitamab and dexamethasone are administered in a dosing regimen comprising at least a first dosing cycle and a second dosing cycle, wherein:
(1) a first dose (C1D1) of 2.5 mg of glofitamab is administered on Day 8 and a second dose (C1D2) of 10 mg of glofitamab is administered on Day 15 of the first dosing cycle; and
(2) a single dose (C2D1) of 30 mg of glofitamab is administered on Day 1 of the second dosing cycle, and wherein the patient receives dexamethasone one day prior to administration of glofitamab, on the same day as glofitamab, and one day after glofitamab.

In one embodiment, the method further comprises administering gemcitabine and oxaliplatin.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and a first dose of gemcitabine and a first dose of oxaliplatin
(b) the second dosing cycle comprises a single dose (C2D1) of 30 mg of glofitamab and a second dose of gemcitabine and a second dose of oxaliplatin, and
wherein dexamethasone is administered one day prior to administration of glofitamab, on the day of the administration of glofitamab, and one day after administration of glofitamab.

In one embodiment, gemcitabine is administered at a dose of 1000 mg/m² oxaliplatin is administered at a dose of 100 mg/m².

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
glofitamab and dexamethasone are administered in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin,
(b) the second dosing cycle comprises a single dose (C2D1) of 30 mg of glofitamab and a dose of 1000 mg/m² of the gemcitabine and a dose of 100 mg/m² of the oxaliplatin,
and wherein the patient receives dexamethasone one day prior to administration of glofitamab, on the same day as glofitamab, and one day after glofitamab.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
glofitamab and dexamethasone are administered in a dosing regimen comprising at least a first dosing cycle and a second dosing cycle, wherein:
(a) a first dose (C1D1) of 2.5 mg of glofitamab is administered on Day 8 and a second dose (C1D2) of 10 mg of glofitamab is administered on Day 15 of the first dosing cycle and a dose of 1000 mg/m² of gemcitabine and a dose of 100 mg/m² of the oxaliplatin on Day 2 of the first dosing cycle; and
(b) a single dose (C2D1) of 30 mg of glofitamab is administered on Day 1 and a dose of 1000 mg/m² of gemcitabine and a dose of 100 mg/m² of the oxaliplatin on Day 1 of the second dosing cycle, and wherein the patient receives dexamethasone one day prior to administration of glofitamab, on the same day as glofitamab, and one day after glofitamab.

In one embodiment, a dose of 1000 mg/m² of the gemcitabine and a dose of 100 mg/m² of oxaliplatin is administered on Day 1 of subsequent dosing cycles.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
(a) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle, and
(b) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab; and
(2) A second dosing cycle, wherein the patient is administered a single dose of 30 mg of glofitamab and an effective dose of gemcitabine, and oxaliplatin.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(4) A first dosing cycle, wherein the patient is administered an effective dose of obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(5) A second dosing cycle, wherein the patient is administered a single dose of 30 mg of glofitamab and an effective dose of gemcitabine, and oxaliplatin.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered a first dose (C1D1) of 2.5 mg of glofitamab and a second dose (C1D2) of 10 mg of glofitamab, and dose of 1000 mg obinutuzumab, 1000 mg/m² gemcitabine, and 100 mg/m² oxaliplatin; and wherein dexamethasone is administered one day prior to the first and second dose of glofitamab, on the same day as the first and second dose of glofitamab and one day after the first and second dose of glofitamab; and
(2) A second dosing cycle, wherein the patient is administered a single dose of 30 mg of glofitamab and a dose of 1000 mg/m² of the gemcitabine and a dose of 100 mg/m² of oxaliplatin.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein dexamethasone is administered one day prior to the first and second dose of glofitamab, on the same day as the first and second dose of glofitamab and one day after the first and second dose of glofitamab; and
(2) A second dosing cycle, wherein the patient is administered a single dose of 30 mg of glofitamab on Day 1 and a dose of 1000 mg/m² of the gemcitabine and a dose of 100 mg/m² of oxaliplatin on Day 1.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered one day prior to the first and second dose of glofitamab, on the same day as the first and second dose of glofitamab and one day after the first and second dose of glofitamab; and
(2) A second dosing cycle, wherein the patient is administered a single dose of 30 mg of glofitamab on Day 1 and a dose of 1000 mg/m² of the gemcitabine and a dose of 100 mg/m² of oxaliplatin on Day 1.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered about 24 hours prior to the first and second dose of glofitamab, about 30-90 or about 60 minutes prior to the first and second dose of glofitamab and about 24 hours after the first and second dose of glofitamab;
(2) A second dosing cycle, wherein the patient is administered a single dose of 30 mg of glofitamab on Day 1 and a dose of 1000 mg/m² of the gemcitabine and a dose of 100 mg/m² of oxaliplatin on Day 1.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:

(1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab and an effective dose of gemcitabine, and oxaliplatin.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered an effective dose of obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab and an effective dose of gemcitabine, and oxaliplatin.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a first dose (C1D1) of 2.5 mg of glofitamab and a second dose (C1D2) of 10 mg of glofitamab, and dose of 1000 mg obinutuzumab, 1000 mg/m² gemcitabine, and 100 mg/m² oxaliplatin; and wherein dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab and a dose of 1000 mg/m² of the gemcitabine and a dose of 100 mg/m² of oxaliplatin.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered about 24 hours prior to the first and second dose of glofitamab, about 30-90 or about 60 minutes prior to the first and second dose of glofitamab and about 24 hours after the first and second dose of glofitamab;
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle,
wherein the method comprises:
   (1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab;
   (2) dosing cycles 2 to 8, wherein the patient is administered a single dose of 30 mg of glofitamab and an effective dose of gemcitabine, and oxaliplatin; and
   (3) dosing cycles 9 to 12, wherein the patient is administered a single dose of 30 mg of glofitamab.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered an effective dose of obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a first dose (C1D1) of 2.5 mg of glofitamab and a second dose (C1D2) of 10 mg of glofitamab, and dose of 1000 mg obinutuzumab, 1000 mg/m² gemcitabine, and 100 mg/m² oxaliplatin; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle; and
(3) dosing cycles 9 to 12, wherein the patient is administered a single dose of 30 mg of glofitamab.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle; and
(3) dosing cycles 9 to 12, wherein the patient is administered a single dose of 30 mg of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, the invention features a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered about 24 hours prior to the first and second dose of glofitamab, about 30-90 or about 60 minutes prior to the first and second dose of glofitamab and about 24 hours after the first and second dose of glofitamab;
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle; and
(3) dosing cycles 9 to 12, wherein the patient is administered a single dose of 30 mg of glofitamab.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one aspect, the invention features a method of reducing the incidence of CRS events in a DLBCL patient population treated with glofitamab, wherein the patients in the population have relapsed after or are refractory to one prior line of therapy and are ineligible for hematopoietic stem cell transplantation, the method, comprising administering to the patients in the patient population glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and
(b) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one aspect, the invention features a method of reducing the incidence of CRS events in a DLBCL patient population treated with glofitamab, wherein the patients in the population have relapsed after or are refractory to one prior line of therapy and are ineligible for hematopoietic stem cell transplantation, the method, comprising administering to the patients in the patient population glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and
(b) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, dexamethasone is administered for the third dose (C2D1) of glofitamab if the patient has experienced CRS with first and or second dose of glofitamab.

In one embodiment, dexamethasone is administered before any subsequent dose of glofitamab if the patient has experienced CRS with any previous 30 mg dose of glofitamab.

In one embodiment, dexamethasone is administered for the first and second dose (C2D1) of glofitamab and not for any subsequent dose.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one aspect, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and
(b) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one aspect, the invention features dexamethasone for use in a method of reducing the likelihood of a CRS event in a CD20-positive B cell proliferative disorder patient treated with glofitamab, comprising administering to the patient glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and
(b) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, dexamethasone is administered for the third dose (C2D1) of glofitamab if the patient has experienced CRS with first and or second dose of glofitamab.

In one embodiment, dexamethasone is administered before any subsequent dose of glofitamab if the patient has experienced CRS with any previous 30 mg dose of glofitamab.

In one embodiment, dexamethasone is administered for the first and second dose (C2D1) of glofitamab and not for any subsequent dose.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one embodiment, the patient has relapsed after or is refractory to one prior line of therapy and is ineligible for hematopoietic stem cell transplantation

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
glofitamab and dexamethasone are administered in a dosing regimen comprising at least a first dosing cycle and a second dosing cycle, wherein:
(1) a first dose (C1D1) of 2.5 mg of glofitamab is administered on Day 8 and a second dose (C1D2) of 10 mg of glofitamab is administered on Day 15 of the first dosing cycle; and
(2) a single dose (C2D1) of 30 mg of glofitamab is administered on Day 1 of the second dosing cycle, and
wherein the patient receives dexamethasone one day prior to administration of glofitamab, on the same day as glofitamab, and one day after glofitamab.

In one embodiment, the method further comprises administering gemcitabine and oxaliplatin.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and a first dose of gemcitabine and a first dose of oxaliplatin
(b) the second dosing cycle comprises a single dose (C2D1) of 30 mg of glofitamab and a second dose of gemcitabine and a second dose of oxaliplatin, and
wherein dexamethasone is administered one day prior to administration of glofitamab, on the day of the administration of glofitamab, and one day after administration of glofitamab.

In one embodiment, gemcitabine is administered at a dose of 1000 mg/m² oxaliplatin is administered at a dose of 100 mg/m².

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:

glofitamab and dexamethasone are administered in a dosing regimen comprising at least a first dosing cycle and a second dosing cycle, wherein:

(a) the first dosing cycle comprises a first dose (C1D1) of 2.5 mg of glofitamab and a second dose (C1D2) of 10 mg of glofitamab and a dose of 1000 mg/m² of the gemcitabine and a dose of 100 mg/m² of the oxaliplatin,
(b) the second dosing cycle comprises a single dose (C2D1) of 30 mg of glofitamab and a dose of 1000 mg/m² of the gemcitabine and a dose of 100 mg/m² of the oxaliplatin,
and wherein the patient receives dexamethasone one day prior to administration of glofitamab, on the same day as glofitamab, and one day after glofitamab.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
glofitamab and dexamethasone are administered in a dosing regimen comprising at least a first dosing cycle and a second dosing cycle, wherein:
(a) a first dose (C1D1) of 2.5 mg of glofitamab is administered on Day 8 and a second dose (C1D2) of 10 mg of glofitamab is administered on Day 15 of the first dosing cycle and a dose of 1000 mg/m² of gemcitabine and a dose of 100 mg/m² of the oxaliplatin on Day 2 of the first dosing cycle; and
(b) a single dose (C2D1) of 30 mg of glofitamab is administered on Day 1 and a dose of 1000 mg/m² of gemcitabine and a dose of 100 mg/m² of the oxaliplatin on Day 1 of the second dosing cycle, and wherein the patient receives dexamethasone one day prior to administration of glofitamab, on the same day as glofitamab, and one day after glofitamab.

In one embodiment, a dose of 1000 mg/m² of the gemcitabine and a dose of 100 mg/m² of oxaliplatin is administered on Day 1 of subsequent dosing cycles.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
(a) a first dose (C1D1) of 2.5 mg of glofitamab is administered on Day 8 and a second dose (C1D2) of 10 mg of glofitamab is administered on Day 15 of the first dosing cycle; and dexamethasone is administered one day prior to administration of the first and second dose of glofitamab, on the same day as the first and second dose of glofitamab and one day after the first and second dose of glofitamab; and a dose of 1000 mg/m² of the gemcitabine and a dose of 100 mg/m² of the oxaliplatin is administered on Day 2 of the first dosing cycle
(b) a single dose (C2D1) of 30 mg of glofitamab is administered on Day 1 of the second dosing cycle and wherein a dose of 1000 mg/m² of the gemcitabine and a dose of 100 mg/m² of oxaliplatin is administered on Day 1 of the second dosing cycle.

In one embodiment, dexamethasone is administered in the second treatment cycle. In one such embodiment, dexamethasone is administered one day prior to administration of the third dose (C2D1) of glofitamab, on the same day as the third dose (C2D1) of glofitamab and one day after the third dose of glofitamab (C2D1).

In one embodiment, dexamethasone is administered one day prior to administration glofitamab, on the same day as glofitamab and one day after glofitamab of any subsequent dose of glofitamab in dosing cycles 1 to 12 if the patient has experienced CRS with any previous 30 mg dose of glofitamab.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle,
wherein the method comprises:
(1) A first dosing cycle, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab; and
(2) A second dosing cycle, wherein the patient is administered a single dose of 30 mg of glofitamab and an effective dose of gemcitabine, and oxaliplatin.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered an effective dose of obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab; and
(2) A second dosing cycle, wherein the patient is administered a single dose of 30 mg of glofitamab and an effective dose of gemcitabine, and oxaliplatin.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered a first dose (C1D1) of 2.5 mg of glofitamab and a second dose (C1D2) of 10 mg of glofitamab, and dose of 1000 mg obinutuzumab, 1000 mg/m² gemcitabine, and 100 mg/m² oxaliplatin; and wherein dexamethasone is administered one day prior to the first and second dose of glofitamab, on the same day as the first and second dose of glofitamab and one day after the first and second dose of glofitamab; and
(2) A second dosing cycle, wherein the patient is administered a single dose of 30 mg of glofitamab and a dose of 1000 mg/m² of the gemcitabine and a dose of 100 mg/m² of oxaliplatin.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein dexamethasone is administered one day prior to the first and second dose of glofitamab, on the same day as the first and second dose of glofitamab and one day after the first and second dose of glofitamab; and
(2) A second dosing cycle, wherein the patient is administered a single dose of 30 mg of glofitamab on Day 1 and a dose of 1000 mg/m² of the gemcitabine and a dose of 100 mg/m² of oxaliplatin on Day 1.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered one day prior to the first and second dose of glofitamab, on the same day as the first and second dose of glofitamab and one day after the first and second dose of glofitamab; and
(2) A second dosing cycle, wherein the patient is administered a single dose of 30 mg of glofitamab on Day 1 and a dose of 1000 mg/m² of the gemcitabine and a dose of 100 mg/m² of oxaliplatin on Day 1.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) A first dosing cycle, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered about 24 hours prior to the first and second dose of glofitamab, about 30-90 or about 60 minutes prior to the first and second dose of glofitamab and about 24 hours after the first and second dose of glofitamab;
(2) A second dosing cycle, wherein the patient is administered a single dose of 30 mg of glofitamab on Day 1 and a dose of 1000 mg/m² of the gemcitabine and a dose of 100 mg/m² of oxaliplatin on Day 1.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab and an effective dose of gemcitabine, and oxaliplatin.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered an effective dose of obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab and an effective dose of gemcitabine, and oxaliplatin.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a first dose (C1D1) of 2.5 mg of glofitamab and a second dose (C1D2) of 10 mg of glofitamab, and dose of 1000 mg obinutuzumab, 1000 mg/m² gemcitabine, and 100 mg/m² oxaliplatin; and wherein dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab and a dose of 1000 mg/m² of the gemcitabine and a dose of 100 mg/m² of oxaliplatin.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered about 24 hours prior to the first and second dose of glofitamab, about 30-90 or about 60 minutes prior to the first and second dose of glofitamab and about 24 hours after the first and second dose of glofitamab;
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a first dose (C1D1) of 2.5 mg of glofitamab and a second dose (C1D2) of 10 mg of glofitamab, and an effective dose of obinutuzumab, gemcitabine, and oxaliplatin; and wherein dexamethasone is administered one day prior to the first and second dose of glofitamab, on the same day as the first and second dose of glofitamab and one day after the first and second dose of glofitamab; and
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered an effective dose of obinutuzumab, glofitamab, gemcitabine, and oxaliplatin, wherein a first dose (C1D1) of 2.5 mg of glofitamab is administered on Day 8 and a second dose (C1D2) of 10 mg of glofitamab is administered on Day 15 of the first dosing cycle; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and an effective dose of gemcitabine, and oxaliplatin; and
(3) dosing cycles 9 to 12, wherein the patient is administered a single dose of 30 mg of glofitamab.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a first dose (C1D1) of 2.5 mg of glofitamab and a second dose (C1D2) of 10 mg of glofitamab, and dose of 1000 mg obinutuzumab, 1000 mg/m² gemcitabine, and 100 mg/m² oxaliplatin; and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab and a dose of 1000 mg/m² of the gemcitabine and a dose of 100 mg/m² of oxaliplatin; and
(3) dosing cycles 9 to 12, wherein the patient is administered a single dose of 30 mg of glofitamab.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle; and
(3) dosing cycles 9 to 12, wherein the patient is administered a single dose of 30 mg of glofitamab.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered one day prior to the first and second dose of glofitamab, on the same day as the first and second dose of glofitamab and one day after the first and second dose of glofitamab;
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 2 of the respective cycle; and
(3) dosing cycles 9 to 12, wherein the patient is administered a single dose of 30 mg of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, the invention features dexamethasone for use in a method of reducing the incidence of CRS events in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein the method comprises:
(1) dosing cycle 1, wherein the patient is administered a dose of 1000 mg obinutuzumab on Day 1, 1000 mg/m² gemcitabine, and 100 mg/m² oxaliplatin on Day 2, a first dose (C1D1) of 2.5 mg of glofitamab on Day 8 and a second dose (C1D2) of 10 mg of glofitamab on Day 15, and wherein 20 mg dexamethasone is administered about 24 hours prior to the first and second dose of glofitamab, about 30-90 or about 60 minutes prior to the first and second dose of glofitamab and about 24 hours after the first and second dose of glofitamab;
(2) dosing cycles 2 to 8, wherein the patient is administered a single dose of 30 mg of glofitamab on Day 1 and a dose of 1000 mg/m² of the gemcitabine and a dose of 100 mg/m² of oxaliplatin on Day 1 of the respective cycle; and
(3) dosing cycles 9 to 12, wherein the patient is administered a single dose of 30 mg of glofitamab.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one aspect, the invention features dexamethasone for use in a method of reducing the incidence of CRS events in a DLBCL patient population treated with glofitamab, wherein the patients in the patient population have relapsed after or is refractory to one prior line of therapy and are ineligible for hematopoietic stem cell transplantation, the method, comprising administering to the patients in the patient population glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and
(b) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one aspect, the invention features dexamethasone for use in a method of reducing the likelihood of a CRS event in a DLBCL patient treated with glofitamab, wherein the patient has relapsed after or is refractory to one prior line of therapy and is ineligible for hematopoietic stem cell transplantation, the method, comprising administering to the patient glofitamab and dexamethasone in a dosing regimen comprising at least a first dosing cycle and a second dosing cycle, wherein:
(a) the first dosing cycle comprises a first dose (C1D1) of 2.5 mg of glofitamab and a second dose (C1D2) of 10 mg of glofitamab; and
(b) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

In one embodiment, dexamethasone is administered at a dose of 20 mg. In one embodiment, dexamethasone is administered about 24 hours prior to administration of glofitamab, about 30-90 or about 60 minutes prior to the glofitamab administration and about 24 hours after administration of glofitamab. In one embodiment, dexamethasone is administered for the first dose (C1D1) of glofitamab and the second dose (C1D2) of glofitamab.

In one embodiment, 20 mg of dexamethasone are administered orally one day prior to the glofitamab administration, 20 mg of dexamethasone are administered intravenously about 30-90 or about 60 minutes prior to the glofitamab administration and 20 mg of dexamethasone are administered orally one day after the glofitamab administration.

In one embodiment, 20 mg of dexamethasone are administered orally about 24 hours prior to the glofitamab administration, 20 mg of dexamethasone are administered intravenously about 30-90 or about 60 minutes prior to the glofitamab administration and 20 mg of dexamethasone are administered orally about 24 hours after the glofitamab administration.

In one embodiment, dexamethasone is administered for the third dose (C2D1) of glofitamab if the patient has experienced CRS with first and or second dose of glofitamab.

In one embodiment, dexamethasone is administered before any subsequent dose of glofitamab if the patient has experienced CRS with any previous 30 mg dose of glofitamab.

In one embodiment, dexamethasone is administered for the first and second dose (C2D1) of glofitamab and not for any subsequent dose.

In one embodiment, the treatment does not cause Grade 3 or higher CRS. In one embodiment the rate of the cytokine release syndrome of any Grade (as defined by the American Society for Transplantation and Cellular Therapy, 2019; ASTCT) is below 40%. In one embodiment, the rate of the cytokine release syndrome of Grade 3 or higher (as defined by the American Society for Transplantation and Cellular Therapy, 2019; ASTCT) is below 1%. In one embodiment, the patient does not need to be hospitalized for the first two cycles of treatment with glofitamab.

In one embodiment, the rate of the cytokine release syndrome of a Grade of 2 or greater (as defined by the American Society for Transplantation and Cellular Therapy, 2019; ASTCT) is less than about 10%, less than about 5%, or less than about 2%.

In one embodiment the cycles are 21 days in length.

In one embodiment, the premedication with a corticosteroid results in superior anti-tumor activity, compared to patients treated without the corticosteroid premedication.

In one embodiment, in the event of a late or prolonged infusion for glofitamab during dosing cycles 2-12 because of a late start in the day or slowed infusion rate due to an IRR or CRS, GemOx is administered on Day 2 of the respective cycle.

In one embodiment, there is an observation period between the end of infusion of glofitamab and before GemOx infusion. In one embodiment said observation period is about 90 minutes long. In one embodiment said observation period is 90 minutes±15 minutes long. At Cycle 3 and beyond, if the participant has not had CRS with prior cycles and has tolerated the preceding glofitamab infusion with no signs or symptoms of CRS, the window of observation is less than about 90 minutes. In one embodiment said observation period is 60 minutes±20 minutes, 60 minutes±15 minutes, or 60 minutes±10 minutes long.

In one embodiment, a first dose (C1D1) of 2.5 mg of glofitamab on Day 8 of the first dosing cycle is infused over 4 hours (±15 minutes), followed by 4 hours (±15 minutes), of monitoring prior to discharge, In one embodiment, the second dose (C1D2) of 10 mg glofitamab on Day 15 of the first dosing cycle is infused over 4 hours (±15 minutes), followed by 4 hours (±15 minutes), of monitoring prior to discharge. In one embodiment, the dose of 30 mg glofitamab on Day 1 of the second dosing cycle is infused over 4 hours (±15 minutes), followed by 90 minutes (±30 minutes), of monitoring prior to discharge. In one embodiment, the dose of 30 mg glofitamab on Day 1 of dosing cycles 2-12 is infused over 4 hours (±15 minutes), followed by 90 minutes (±30 minutes), of monitoring prior to discharge. In one embodiment, the dose of 30 mg on Day 1 of dosing cycle 3-12 is infused over 2 hours (±15 minutes) and the window of observation is less than 90 minutes.

D. Pretreatment or Management of CRS Related Symptoms with Tocilizumab

CRS is associated with high IL-6 levels (Panelli et al., J Transl Med, 2:17, 2004; Lee et al., Blood, 124:188-195, 2014; Doessegger and Banholzer, Clin Transl Immunology, 4: e39, 2015), and IL-6 correlates with the severity of CRS, with patients who experience severe or life-threatening CRS (NCI CTCAE Grades 4 or 5) having much higher IL-6 levels compared with their counterparts who do not experience CRS or experience milder CRS reactions (NCI CTCAE Grades 0-3) (Chen et al., J Immunol Methods, 434:1-8, 2016).

Tocilizumab (ACTEMRA®/ROACTEMRA®) is a recombinant, humanized, anti-human monoclonal antibody directed against soluble and membrane-bound IL-6R, which inhibits IL-6 mediated signaling (see, e.g., WO 1992/019579, which is incorporated herein by reference in its entirety). Tocilizumab has been approved by the U.S. Food and Drug Administration for the treatment of severe or life-threatening CAR-T cell-induced CRS in adults and in pediatric patients 2 years of age and older. Initial clinical data (Locke et al., Blood, 130:1547, 2017) suggests that tocilizumab prophylaxis may reduce the severity of CAR-T cell-induced CRS by blocking IL-6 receptors from signaling prior to cytokine release. Consequently, tocilizumab premedication may also reduce the frequency or lower the severity of CRS associated with glofitamab therapy. Other anti-IL-6R antibodies that could be used in combination with tocilizumab include sarilumab, vobarilizumab (ALX-0061), SA-237, and variants thereof.

In some aspects, an effective amount of tocilizumab is administered as a premedication, e.g., is administered to the patient prior to the administration of glofitamab. Administration of tocilizumab as a premedication may reduce the frequency or severity of CRS. In some aspects, tocilizumab is administered as a premedication in Cycle 1, e.g., is administered prior to a first dose (C1D1), a second dose (C1D2), and/or a third dose (C1D3) of glofitamab. In some aspects, tocilizumab is administered intravenously to the patient as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, tocilizumab is administered intravenously to the patient as a single dose of about 8 mg/kg. Other anti-IL-6R antibodies that could be used in combination with tocilizumab include sarilumab, vobarilizumab (ALX-0061), SA-237, and variants thereof.

For example, in one aspect, glofitamab is co-administered with tocilizumab (ACTEMRA®/ROACTEMRA®), wherein the patient is first administered with tocilizumab (ACTEMRA®/ROACTEMRA®) and then separately administered with glofitamab (e.g., the patient is pre-treated with tocilizumab (ACTEMRA®/ROACTEMRA®)).

In another aspect, tocilizumab is administered to treat or alleviate symptoms associated with CRS in patients treated with glofitamab. If the patient has a grade 2 or higher CRS event in the presence of extensive comorbidities following administration of glofitamab, the method may further include administering to the patient a first dose of an IL-6R antagonist (e.g., an anti-IL-6R antibody, e.g., tocilizumab (ACTEMRA®/ROACTEMRA®)) to manage the grade 2 or higher CRS event while suspending treatment with glofitamab. In some instances, the first dose of tocilizumab is administered intravenously to the patient at a dose of about 8 mg/kg. Other anti-IL-6R antibodies that could be used in combination with tocilizumab include sarilumab, vobarilizumab (ALX-0061), SA-237, and variants thereof. In some instances, if the grade 2 or higher CRS event resolves to a grade ≤1 CRS event within two weeks, the method further includes resuming treatment with glofitamab at a reduced dose. In some instances, the reduced dose is 50% of the initial infusion rate of the previous cycle if the event occurred during or within 24 hours of the infusion. If, on the other hand, the grade 2 or higher CRS event does not resolve or worsens to a grade ≥3 CRS event within 24 hours of treating the symptoms of the grade 2 or higher CRS event, the method may further include administering to the patient one or more (e.g., one, two, three, four, or five or more) additional doses of an IL-6R antagonist (e.g., an anti-IL-6R antibody, e.g., tocilizumab) to manage the grade 2 or grade ≥3 CRS event. In some particular instances, the grade 2 or higher CRS event does not resolve or worsens to a grade ≥3 CRS event within 24 hours of treating the symptoms of the grade 2 or higher CRS event, and the method may further include administering to the patient one or more additional doses of tocilizumab to manage the grade 2 or grade ≥3 CRS event. In some instances, the one or more additional doses of tocilizumab is administered intravenously to the patient at a dose of about 1 mg/kg to about 15 mg/kg, e.g., about 4 mg/kg to about 10 mg/kg, e.g., about 6 mg/kg to about 10 mg/kg, e.g., about 8 mg/kg.

### E. Other Pretreatments for CRS Risk Mitigation

In one embodiment, the treatment regimen provided herein further comprises administration of premedication prior to the administration of glofitamab. In one embodiment the premedication comprises paracetamol/acetaminophen, and/or an anti-histamine (such as, e.g., diphenhydramine). In one embodiment, the premedication is administered at least 60 minutes (e.g., at least 2, 3, 4, 5, 6, 7, 8, 9, 10, 11, 12, 18, 24, 36, 48 hours, or more) prior to the administration of glofitamab. In one embodiment, the treatment regimen further comprises administration of premedication prior to the administration of glofitamab. In embodiment the premedication comprises an anti-pyretic (such as, e.g., paracetamol/acetaminophen), and/or an anti-histamine (such as, e.g., diphenhydramine). In one embodiment, the premedication is administered at least 60 minutes (e.g., at least 2, 3, 4, 5, 6, 7, 8, 9, 10, 11, 12, 18, 24, 36, 48 hours, or more) prior to the administration of glofitamab. In one embodiment, the premedication is administered at least 60 minutes (e.g., at least 2, 3, 4, 5, 6, 7, 8, 9, 10, 11, 12, 18, 24, 36, 48 hours, or more) prior to each administration of glofitamab. In another embodiment, pre-medication is administered before the first dose (C1D1) and second dose (C1D2) of the first cycle, before the first dose of the second (C2D1) and third (C3D1) cycle and may be optional for subsequent cycles where the target dose has been reached and tolerated for two doses for patients with no CRS in previous cycles.

In one embodiment, the premedication is administered at least 60 minutes (e.g., at least 2, 3, 4, 5, 6, 7, 8, 9, 10, 11, 12, 18, 24, 36, 48 hours, or more) prior to the administration of the pretreatment with the anti-CD20 antibody, particularly obinutuzumab.

In one embodiment, corticosteroids are administered to manage any relevant adverse events arising after administration of glofitamab.

A further aspect of the present invention relates to the invention as described hereinbefore.

### EXAMPLES

The following are examples of methods and compositions of the invention. It is understood that various other embodiments may be practiced, given the general description provided above.

### Example 1. A Phase III, Open-Label, Multicenter, Randomized Study Evaluating the Efficacy and Safety of Glofitamab in Combination with Gemcitabine Plus Oxaliplatin Versus Rituximab in Combination with Gemcitabine and Oxaliplatin in Patients with Relapsed/Refractory Diffuse Large B-Cell Lymphoma

### Objectives

This study evaluated the efficacy and safety of glofitamab in combination with gemcitabine plus oxaliplatin (Glofit-GemOx) compared with rituximab in combination with gemcitabine plus oxaliplatin (R-GemOx) in patients with R/R DLBCL. Specific objectives and corresponding endpoints for the study are outlined below in Table 5.

TABLE 5
Objectives and Endpoints

| | Corresponding Endpoints |
|---|---|
| Objectives | |

Primary Efficacy Objective:

| | |
|---|---|
| To evaluate the preliminary efficacy of Glofit- | Overall survival (OS), defined as the time from |
| GemOx compared with R-GemOx | randomization to date of death from any cause |

Secondary Efficacy Objective:

| | |
|---|---|
| To evaluate the efficacy of Glofit-GemOx | Note: All response assessments were based on |
| compared with R-GemOx | the 2014 Lugano Response Criteria. |
| | Progression-free survival (PFS), defined |
| | as the time from randomization to the first |
| | occurrence of disease progression or |
| | death from any cause, whichever occurs |
| | first |
| | Complete response (CR) rate, defined as |
| | the proportion of patients whose best |
| | overall response is a CR on positron |
| | emission tomography/computed |
| | tomography (PET/CT) during the study |
| | Objective response rate (ORR), defined |
| | as the proportion of patients whose best |
| | overall response is a partial response |
| | (PR) or a CR during the study |
| | Duration of objective response, defined as |
| | the time from the first occurrence of a |
| | documented objective response (CR or |
| | PR) to disease progression, or death from |
| | any cause, whichever occurs first |
| | Duration of CR, defined as the time from |
| | the first occurrence of a documented CR |
| | to disease progression, or death from any |
| | cause, whichever occurs first |
| | Time to deterioration in physical |
| | functioning and fatigue, as measured by |
| | the European Organisation for Research |
| | and Treatment of Cancer Quality of |
| | Life-Core 30 Questionnaire (EORTC |
| | QLQ-C30) and in lymphoma symptoms, |
| | as measured by the Functional |
| | Assessment of Cancer |
| | Therapy-Lymphoma subscale (FACT- |
| | Lym LymS) |
| Exploratory Efficacy Objective | |
| To evaluate the efficacy of Glofit-GemOx | Descriptive summary statistics of patient- |
| compared with R-GemOx | reported outcomes and the change from |
| | baseline by treatment arm at each |
| | assessment for the following: |
| | All remaining scales of the EORTC QLQ-C30 |
| | FACT-Lym LymS subscale |
| | Characterization of patients who become |
| | hematopoietic stem cell transplant |
| | (HSCT) candidates after study therapy |
| | and are treated with autologous or |
| | allogeneic HSCT, including: |
| | Incidence of autologous and allogeneic HSCT |
| | after study therapy |
| | Survival post-HSCT, defined as the time from |
| | date of transplantation to date of death from any |
| | cause |
| | Characterization of patients who receive |
| | chimeric antigen receptor (CAR) T-cell |
| | therapy after study therapy and are |
| | treated with CAR T-cell therapy, including: |
| | Incidence of treatment with CAR T-cell therapy |
| | Survival post-CAR-T-cell therapy, defined as the |
| | time from date of CAR T-cell infusion to date of |
| | death from any cause |
| Safety Objective | |
| To evaluate the safety and tolerability of Glofit- | Incidence and severity of adverse events, |
| GemOx compared with R-GemOx | with severity determined according to the |
| | National Cancer Institute Common |
| | Toxicity Criteria for Adverse Events, |
| | Version 5.0 (NCI CTCAE v5.0), including |
| | CRS, with severity determined according |
| | to the American Society for |
| | Transplantation and Cellular Therapy |
| | (ASTCT) CRS grading criteria |
| | Change from baseline in targeted vital signs |
| | Change from baseline in targeted clinical |
| | laboratory test results |
| | Tolerability, as assessed by dose |
| | interruptions, dose reductions, and dose |
| | intensity, and study treatment |
| | discontinuation because of adverse |
| | events |
| Safety Exploratory Objectives | |
| To evaluate the post-transplant Day 100 | Day 100 non-relapse mortality, defined as |
| non-relapse mortality for patients who become | the incidence of death not related to disease |
| HSCT candidates after study therapy and are | progression within 100 days of transplantation |
| treated with autologous or allogeneic HSCT | Day 100 non-relapse mortality, defined as |
| To evaluate the post-CAR-T infusion Day | the incident of death not related to disease |
| 100 non-relapse mortality for patients who | progression within 100 days of CAR T-cell therapy |
| become candidates for and are treated with CAR | Incidence and severity of CRS |
| T-cell therapy after study therapy | Incidence and severity of immune |
| To evaluate the safety in patients who are | effector cell-associated neurotoxicity |
| treated with CAR T-cell therapy after study | syndrome (ICANS) |
| treatment | |

Abbreviations: see table 8 below.

### Study Design

Described herein is a Phase III, open-label, multicenter, randomized controlled trial in patients with R/R DLBCL. Eligible patients were randomized in a 2:1 ratio to receive either Glofit-GemOx or R-GemOx. Patients in this trial were stratified at the time of randomization for the following factors:
Number of previous lines of systemic therapy for DLBCL (1 vs. ≥2).
CAR T-cell plus bridging therapy was counted as one line of therapy.
Local therapies (e.g., radiotherapy) was not considered as a line of therapy.
Outcome of last systemic therapy (relapsed vs. refractory).
Relapsed disease in this study was defined as disease that has recurred following a response that lasted ≥6 months after completion of the last line of therapy.
Refractory disease was defined as disease that did not respond to or that progressed ≤6 months after completion of the last line of therapy.

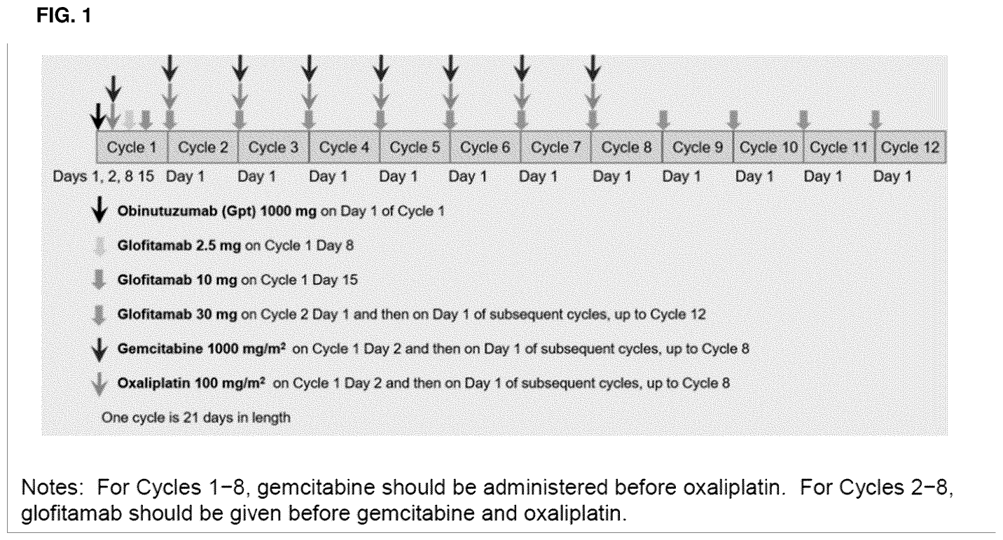

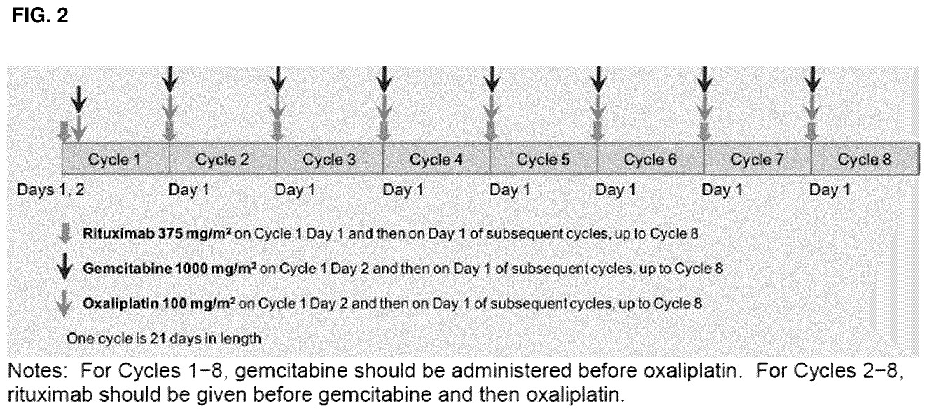

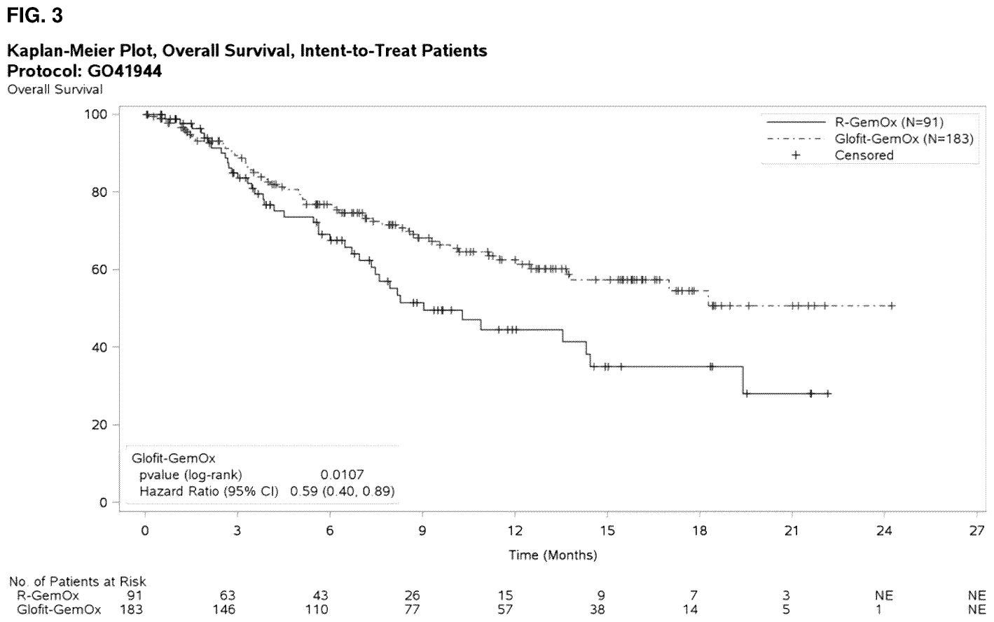

Patients in the Glofit-GemOx arm received a single intravenous (IV) dose of obinutuzumab pretreatment 7 days before the first dose of glofitamab, then up to 8 cycles of glofitamab in combination with gemcitabine plus oxaliplatin, followed by up to 4 cycles of glofitamab monotherapy to complete up to 12 cycles of glofitamab, with each cycle being 21 days (i.e., every 3 weeks). Patients in the R-GemOx arm received rituximab in combination with gemcitabine plus oxaliplatin for up to 8 cycles. See FIG. 1 and FIG. 2 for dosing regimens for the Glofit-GemOx and R-GemOx treatment arms, respectively.

Patients who discontinued last line of therapy before sufficient time for response assessment (for example, due to toxicity) were assessed for refractoriness based on the previous line of therapy. The study utilized a group sequential design based on conditional power observed at an interim analysis (70% of the events). After the interim analysis, a decision was made on the basis of the independent Data Monitoring Committee's (iDMC) recommendation to release the efficacy data and analyze for superior efficacy.

Safety was assessed by adverse events (with severity determined per the NCI CTCAE v5.0, except CRS severity which was determined per the ASTCT CRS grading criteria), clinical laboratory test results, and vital signs. Response assessments was according to the 2014 Lugano Response Criteria, as assessed on PET/CT scans.

### Target Population

### Inclusion Criteria

Patients must meet the following criteria for study entry:
Histologically confirmed diffuse large B-cell lymphoma, not otherwise specified
R/R disease, defined as follows:
   Relapsed: disease that has recurred following a response that lasted ≥6 months after completion of the last line of therapy
   Refractory: disease that did not respond to or that progressed ≤6 months after completion of the last line of therapy

Patients who discontinued last line of therapy before sufficient time for response assessment (for example, due to toxicity) were assessed for refractoriness based on the previous line of therapy.
At least one (≥1) line of prior systemic therapy
Patients may have undergone autologous HSCT prior to recruitment
CAR T-cell plus bridging therapy was counted as one line of therapy.
Local therapies (e.g., radiotherapy) were not considered as lines of therapy.
Transplant ineligibility
Patients who have failed only one prior line of therapy must not have been a candidate for high-dose chemotherapy followed by autologous stem cell transplant by meeting at least one of the following criteria:
   Left ventricular ejection fraction ≤40%
   Creatinine clearance or glomerular filtration rate ≤45 mL/min
   Eastern Cooperative Oncology Group (ECOG) Performance Status of ≥2
   Age ≥70 years
   Patient refused high-dose chemotherapy and/or transplant
   Patient had insufficient response to pre-transplant chemotherapy to be able to proceed to transplant
   Other comorbidities or criteria that preclude use of transplant.
At least one bi-dimensionally measurable (≥1.5 cm) nodal lesion, or one bi-dimensionally measurable (≥1 cm) extranodal lesion, as measured on CT scan
ECOG Performance Status of 0, 1, or 2
Adequate hematologic function (unless attributable to the underlying disease, as established by extensive bone marrow involvement or associated with hypersplenism secondary to the involvement of the spleen by DLBCL per the investigator), defined as follows:

$$\begin{matrix}Hemoglobin \geq 9. g / dL ( \geq 90 g / L ) \\ ANC \geq 1. \times {10}_{9} / L ( \geq 1 \; 000 / μL ) \\ Platelet count \geq 75 \times {10}_{9} / L ( \geq 75 , 000 / μL )\end{matrix}$$

Adequate renal function, defined as an estimated creatinine clearance ≥30 mL/min

### Exclusion Criteria

Patients who meet any of the following criteria were excluded from study entry:
Patient has failed only one prior line of therapy and is a candidate for stem cell transplantation.
History of transformation of indolent disease to DLBCL.
High-grade B-cell lymphoma with MYC and BCL2 and/or BCL6 rearrangements, and high-grade B-cell lymphoma not otherwise specified, as defined by 2016 WHO guidelines

Patients classified as “double-hit” lymphoma with known MYC and BCL2 and/or BCL6 rearrangements are excluded. Patients with DLBCL characterized as “double expressors” without the above rearrangements were not excluded.
Primary mediastinal B-cell lymphoma.
History of severe allergic or anaphylactic reactions to humanized or murine monoclonal antibodies (or recombinant antibody-related fusion proteins) or known sensitivity or allergy to murine products
Contraindication to obinutuzumab, rituximab, gemcitabine or oxaliplatin, or tocilizumab.
Prior treatment with glofitamab or other bispecific antibodies targeting both CD20 and CD3.
Prior treatment with R-GemOx or GemOx.
Peripheral neuropathy assessed to be Grade >1 according to NCI CTCAE v5.0 at enrollment.
Any of the following abnormal laboratory values, unless abnormal laboratory values were associated with the underlying lymphoma per the investigator:

$$\begin{matrix}AST or ALT \geq 2.5 \times upper limit of normal ( ULN ) \\ Total bilirubin \geq 1.5 \times ULN\end{matrix}$$

Patients with documented Gilbert disease may have been enrolled if the total bilirubin is ≤3×ULN.
INR or PT >1.5×ULN, or Quick percentage <70% (if Quick percentage used in lieu of time-based units for reporting PT), in the absence of therapeutic anticoagulation
PTT or aPTT >1.5×ULN in the absence of therapeutic anticoagulation or a lupus anticoagulant

Enrollment of patients with platinum-refractory disease was limited to approximately 20% of the total number of randomized patients. Platinum-refractory is defined as disease that did not respond to or that progressed <6 months after treatment with platinum-containing regimens, with or without rituximab, including ICE (ifosfamide, carboplatin, etoposide), DHAP (dexamethasone, cytarabine, cisplatin), DHAC (dexamethasone, cytarabine, carboplatin), GDP (gemcitabine, dexamethasone, cisplatin or carboplatin), or other intensive platinum-containing regimens intended as pre-ASCT salvage therapy. Enrollment of patients who have received more than one prior line of therapy was limited to approximately 65% of the total number of randomized patients.

### Treatment Assignment

Patients were randomly assigned to one of two treatment arms: Glofit-GemOx or R-GemOx. Randomization occurred via interactive voice or web-based response system (IxRS) in a 2:1 ratio. Patients were stratified at the time of randomization for the following factors:
Number of previous lines of systemic therapy for DLBCL (1 (e.g., 2L) vs. ≥2 (e.g., 3L+))
   CAR T-cell plus bridging therapy was counted as one line of therapy.
   Local therapies (e.g., radiotherapy) was not considered as a line of therapy.
Outcome of last systemic therapy (relapsed vs. refractory
   Relapsed disease in this study is defined as disease that has recurred following a response that lasted ≥6 months after completion of the last line of therapy.
   Refractory disease is defined as disease that did not respond to or that progressed <6 months after completion of the last line of therapy.

Patients who discontinued last line of therapy before sufficient time for response assessment (for example, due to toxicity) were assessed for refractoriness based on the previous line of therapy.

### Test Products (Investigational Drugs)

### Glofit-GemOx Arm

### Obinutuzumab

A single dose of obinutuzumab 1000 mg pretreatment was administered intravenously on Day 1 of Cycle 1 (7 days prior to the first administration of glofitamab).

### Glofitamab

Glofitamab is a bispecific antibody that binds to CD20 and CD3 comprising two Fab molecules which specifically bind to CD20 comprising the following six hypervariable regions (HVRs):
HVR-H1: YSWIN (SEQ ID NO: 1); HVR-H2: RIFPGDGDTDYNGKFKG (SEQ ID NO:2); HVR-H3: NVFDGYWLVY (SEQ ID NO:3); HVR-L1: RSSKSLLHSNGITYLY (SEQ ID NO: 4); HVR-L2: QMSNLVS (SEQ ID NO: 5); and HVR-L3: AQNLELPYT (SEQ ID NO: 6) and one Fab molecule which specifically binds to CD3 comprising the following six hypervariable regions (HVRs): HVR-H1: TYAMN (SEQ ID NO: 9); HVR-H2: RIRSKYNNYATYYADSVKG (SEQ ID NO:10); HVR-H3: HGNFGNSYVSWFAY (SEQ ID NO: 11); HVR-L1: GSSTGAVTTSNYAN (SEQ ID NO: 12); HVR-L2: GTNKRAP (SEQ ID NO: 13); and HVR-L3: ALWYSNLWV (SEQ ID NO: 14).

Glofitamab was administered on a step-up dosing schedule starting on Day 8 of Cycle 1 (2.5 mg), Day 15 of Cycle 1 (10 mg), followed by 30 mg on Day 1 of Cycles 2-12, with each cycle being 21 days (i.e., every 3 weeks).

In the event of a late or prolonged infusion for glofitamab during Cycles 2-12 because of a late start in the day or slowed infusion rate due to an infusion-related reaction (IRR) or CRS, it was allowed to administer GemOx on the following day or as per local practice. There was a 90-minute observation period between the end of infusion of glofitamab and before GemOx infusion. At Cycle 3 and beyond, if the patient has not had CRS with prior cycles, and has tolerated the preceding glofitamab infusion with no signs or symptoms of CRS, the window of observation was shortened based on the discretion of the investigator.

Patients were hospitalized at least overnight after the first infusion of glofitamab on Day 8 of Cycle 1. Patients with an event of Grade ≥2 CRS associated with the preceding dose were hospitalized at least overnight for the next dose of glofitamab. Investigators could hospitalize a patient following any glofitamab dose for close monitoring if indicated based on their clinical judgment. Hospitalization was considered following the second dose of glofitamab for patients who lack support for close monitoring at home. The frequency and duration of hospitalization was reduced based on iDMC recommendation after review of the emerging safety profile of study treatment.

Premedication with dexamethasone 20 mg IV was administered 1 hour prior to the administration of glofitamab; premedication with acetaminophen or paracetamol (500-1000 mg) and an antihistamine, such as diphenhydramine (50 mg), was administered approximately 30 minutes prior to the start of the infusion. Additional details for premedication are described below in Table 6.

Glofitamab was administered to well-hydrated patients. Patients at risk of TLS received TLS prophylaxis.

Initially, glofitamab was administered over 4 hours (±15 minutes) on Days 8 and 15 of Cycle 1 and on Day 1 of Cycle 2. Following each glofitamab dose, patients was observed at least 90 minutes for fever, chills, rigors, hypotension, hypoxia, nausea, or other signs and symptoms of CRS. At Cycle 3 and beyond, if the patient had not had CRS with prior cycles, and had tolerated the preceding glofitamab infusion with no signs or symptoms of CRS, the window of observation was shortened based on the discretion of the investigator. For patients who developed CRS with onset of associated signs/symptoms during glofitamab infusion, infusion was discontinued immediately.

For patients who at an increased risk of CRS and patients who experienced CRS with their previous dose of glofitamab or who were in the investigator's judgment at increased risk of recurrent CRS with subsequent doses, the time of infusion was extended to up to 8 hours. In the absence of infusion-related adverse events or CRS with their previous dose of glofitamab, the infusion time of glofitamab in Cycles 3 and beyond was reduced to 2 hours (±15 minutes), at the discretion of the investigator. Alternately, patients who were, in the investigator's judgment, at an increased risk of recurrent CRS with subsequent doses, had the time of infusion extended to up to 8 hours.

Dexamethasone premedication was optional at later cycles based on investigator's assessment for patients who tolerated two 30 mg doses of glofitamab without experiencing CRS. However, if the patient experienced CRS, premedication with dexamethasone was required for subsequent glofitamab doses until no additional CRS events were observed. Patients were hospitalized at least overnight after the infusion of glofitamab on Day 8 of Cycle 1. Patients with an event of Grade ≥2 CRS associated with the preceding dose of glofitamab were hospitalized at least overnight for the next glofitamab dose. Investigators hospitalized a patient following any glofitamab dose for close monitoring if indicated based on their clinical judgment. Hospitalization was considered following the second dose of glofitamab for patients who lacked support for close monitoring at home.

In case of infusion-associated adverse events in patients, the signs and symptoms were fully resolved before the patient was discharged.

TABLE 6
Pre-medications before obinutuzumab and glofitamab Infusions

| | Patients Requiring | | |
|---|---|---|---|
| Timepoint | Premedication | Premedication | Administration |
| Obinutuzumab ^{a} | All patients | Hydration | Fluid intake of approximately 2-3 |
| Cycle 1, Day 1 | | | L/day starting 24-48 hours prior |
| | | | to the first dose of study |
| | | | treatment |
| | | IV glucocorticoids ^{b} | At least 60 minutes prior to |
| | | | obinutuzumab infusion |
| | | Oral or IV analgesic | At least 30 minutes prior to |
| | | or anti-pyretic | obinutuzumab infusion |
| | | medicine | |
| | | Oral or IV | At least 60 minutes prior to |
| | | antihistamine ^{c} | obinutuzumab infusion |
| | Patients at risk of TLS | Allopurinol or | 48-72 hours prior to study |
| | (e.g., because of bulky | suitable alternative, | treatment |
| | disease or renal | such as rasburicase, | |
| | impairment creatinine | along with adequate | |
| | clearance <70 mL/min) | hydration | |
| Glofitamab ^{e, f} | All patients | IV | At least 60 minutes prior to the |
| Cycle 1, Day 8 | | dexamethasone ^{d, e} | first dose of stuy treatment |
| and onward; all | | Oral or IV analgesic | At least 30 minutes prior to |
| doses | | or anti-pyretic | glofitamab infusion |
| | | medicine | |
| | | Oral or IV | At least 30 minutes prior to |
| | | antihistamine ^{c} | glofitamab infusion |
| | Patients at risk of TLS | Allopurinol or | 48-72 hours prior to study |
| | (e.g., because of bulky | suitable alternative, | treatment |
| | disease or renal | such as rasburicase, | |
| | impairment creatinine | along with adequate | |
| | clearance <70 mL/min) | hydration | |

CRS = cytokine release syndrome; PO = orally; TLS = tumor lysis syndrome.
^{a} Closely monitor patients during the entire infusion. Infusion reactions within 24 hours of receiving obinutuzumab have occurred. Obinutuzumab should be administered to well-hydrated patients.
^{b} IV glucocorticoid prior to obinutuzumab: dexamethasone 20 mg IV or methylprednisolone 80 mg IV.
^{c} For example, 50 mg diphenhydramine (unless contraindicated).
^{d} Administer dexamethasone 20 mg IV prior to glofitamab. If dexamethasone is not available, or the patient has an intolerance to dexamethasone, alternate corticosteroid (methylprednisolone 80 mg IV, prednisone 100 mg PO, or prednisolone 100 mg PO or IV) may be used for glofitamab premedication, after consulting with the Medical Monitor.
^{e} Optional at later cycles based on investigator's assessment for patients that have tolerated two 30 mg doses of glofitamab without experiencing CRS.
^{f} All glofitamab doses should be administered to well-hydrated patients. Closely monitor patients during the entire infusion.

### R-GemOx Arm

### Rituximab

On Day 1 of each 21-day cycle, 375 mg/m² rituximab was administered to patients by IV infusion for eight cycles. Only rituximab was administered on Day 1 of Cycle 1, followed by gemcitabine and oxaliplatin on Day 2 of Cycle 1. Rituximab was administered before gemcitabine and oxaliplatin on the same day during Cycles 2-8; in the event of a late or prolonged infusion for rituximab owing to starting late in the day, or slowed infusion rate because of an IRR, it was allowed to administer GemOx on the following day.

During the treatment period, rituximab was administered to patients in a setting where full emergency resuscitation facilities are immediately available. Patients were under close supervision of the investigator at all times. The local prescribing information provided guidance for the management of adverse events, including IRRs. Rituximab was administered before gemcitabine and oxaliplatin. Once the rituximab infusion was completed, patients were observed for 30 minutes before the start of the other infusions. The infusion of rituximab was split over 2 days if the patient was at increased risk for an IRR (high tumor burden or high peripheral lymphocyte count). For patients who experienced an adverse event during a rituximab infusion, administration of rituximab and gemcitabine and oxaliplatin was continued on the following day, if required.

If a dose of rituximab was split over 2 days, both infusions had to occur with appropriate premedication (including prednisone) and at the first infusion rate. All rituximab infusions were administered to patients after premedication with acetaminophen (e.g., 650-1000 mg) and an antihistamine such as diphenhydramine hydrochloride (50-100 mg) 30-60 minutes before starting each infusion (unless contraindicated). A dose of glucocorticoid (methylprednisolone 80 mg IV or equivalent dose of dexamethasone [20 mg IV], prednisone [100 mg] or prednisolone [100 mg]) was allowed at the investigator's discretion. For patients who did not experience infusion-related symptoms with their previous infusion, premedication at subsequent infusions were omitted at the investigator's discretion. Rituximab infusions were administered according to the instructions outlined in Table 7. If a patient tolerated the first cycle of rituximab without significant IRRs, rituximab was administered as a rapid infusion (over 60-90 minutes).

TABLE 7
Administration of Rituximab

| First Infusion (Day 1) | Subsequent Infusions |
|---|---|
| Begin infusion at an initial rate of 50 mg/hr. | If the patient experienced an infusion-related |
| If no infusion-related or hypersensitivity | or hypersensitivity reaction during the prior |
| reaction occurs, increase the infusion rate in | infusion, begin infusion at an initial rate of |
| 50-mg/hr increments every 30 minutes, to a | 50 mg/hr and follow instructions for the first |
| maximum of 400 mg/hr. | infusion. |
| If an infusion-related reaction develops, stop | If the patient tolerated the prior infusion well |
| or slow the infusion. Administer | (defined as an absence of Grade 2 reactions |
| infusion-reaction medications and supportive | during a final infusion rate of ≥100 mg/hr), |
| care in accordance with institutional | begin the infusion at a rate of 100 mg/hr. |
| guidelines. If the reaction resolves, resume | If no infusion-related reaction occurs, increase |
| the infusion at a 50% reduction in rate (i.e., | the infusion rate in 100 mg/hr increments |
| 50% of rate being used at the time that the | every 30 minutes, to a maximum of 400 |
| reaction occurred). | mg/hr. |
| | If an infusion reaction develops, stop or slow |
| | the infusion. Administer infusion-reaction |
| | medications and supportive care in |
| | accordance with institutional guidelines. If the |
| | reaction resolves, resume the infusion at a |
| | 50% reduction in rate (i.e., 50% of rate being |
| | used at the time that the reaction occurred). |

### Tocilizumab

Tocilizumab was only administered to those patients who experienced a CRS event for which tocilizumab is indicated (a rescue IMP). For patients requiring treatment of CRS, patients received tocilizumab by IV infusion.

Patients who weigh ≥30 kg received 8 mg/kg tocilizumab and patients who weigh ≤30 kg received 12 mg/kg tocilizumab. Doses exceeding 800 mg per infusion were not recommended. Treatment was repeated every 8 hours as necessary (for a maximum of four doses).

### Non-Investigational Medicinal Products

Gemcitabine and oxaliplatin are considered as standard of care in many countries for the treatment of patients with R/R DLBCL and are non-IMPs for this study.

### Gemcitabine

Gemcitabine was administered intravenously to patients in both treatment arms at 1000 mg/m² on Day 2 of Cycle 1. Starting at Cycle 2, gemcitabine was given on Day 1 or 2 (per local practice) of each 21-day cycle (Cycles 2-8). Gemcitabine was administered before oxaliplatin on the same day.

### Oxaliplatin

Oxaliplatin was administered intravenously to patients in both treatment arms at 100 mg/m² on Day 2 of Cycle 1. Starting at Cycle 2, gemcitabine was given on Day 1 or 2 (per local practice) of each 21-day cycle (Cycles 2-8). Oxaliplatin was administered after gemcitabine on the same day.

### Required Therapy

### Prophylaxis for Neutropenia

All patients received granulocyte colony-stimulating factor (G-CSF) as primary prophylaxis during Cycles 1 and 2 of GemOx treatment. G-CSF was started 1-2 days after GemOx infusion. In subsequent cycles the use of G-CSF was optional in patients who did not have therapy delays due to neutropenia. Dosing of G-CSF was otherwise followed each site's institutional standards. At the investigator's discretion, G-CSF was not required in patients with a history of hyperleukocytosis or significant pain associated with G-CSF, or in patients who developed hyperleukocytosis with study therapy.

### Prophylaxis for Tumor Lysis Syndrome

Patients with high tumor burden and/or considered by the investigator to be at risk for tumor lysis received tumor lysis prophylaxis prior to the initiation of treatment (Coiffier et al. 2008). All patients were advised to be well hydrated. Starting 2 days prior to the first dose of study treatment, the patients were advised to maintain a fluid intake of approximately 2-3 L/day. In addition, all patients with high tumor burden and considered to be at risk for tumor lysis were treated with 300 mg/day of allopurinol orally or a suitable alternative treatment (e.g., rasburicase), starting 48-72 hours prior to Day 1 of Cycle 1. Patients continued to receive repeated prophylaxis, if deemed appropriate by the investigator, and adequate hydration prior to each subsequent cycle of treatment.

### Permitted Therapy

In general, investigators managed a patient's care with supportive therapies as clinically indicated, per local standard practice. Patients who use oral contraceptives, hormone-replacement therapy, or other maintenance therapy continued their use.

### Tumor and Response Evaluations

All evaluable or measurable disease was documented at screening and reassessed at each subsequent tumor evaluation. Response was assessed by the investigator, on the basis of physical examinations, diagnostic CT scans (or magnetic resonance imaging [MRI] scans), and PET/CT scans, and by the IRC, with response determined according to the 2014 Lugano Response Criteria.

Bone marrow biopsies were not routinely required for response assessment but were indicated in specific situations per the 2014 Lugano Response Criteria (e.g., CT-only-based response assessment or when residual uptake is seen on PET/CT scan).

### Radiographic Assessments

The same radiographic assessment modality was used for all response evaluations in order to ensure consistency across different timepoints. PET/CT scans in conjunction with diagnostic CT scans were obtained in this study. PET/CT scans included the base of the skull to mid-thigh. A full-body PET/CT scan was performed when clinically appropriate. CT and PET/CT scans were required at screening, after Cycle 4 (i.e., Days 15-21 of Cycle 4) of combination therapy, after Cycle 8 (Days 15-21 of Cycle 8), and 6-8 weeks after Day 1 of Cycle 12 in the Glofit-GemOx arm, or 6-8 weeks after the final dose of study treatment for patients who discontinued study treatment prematurely. If local practice prohibited obtaining both assessments after Cycle 4, a PET/CT scan alone (preferred) or CT scan alone was obtained at these timepoints. The end-of-treatment response assessments were based on scans performed between Days 15 and 21 of Cycle 8 (for the R-GemOx arm) and 6-8 weeks after Day 1 of Cycle 12 (for the Glofit-GemOx arm), in patients who completed study treatment; in patients who discontinued study treatment before completing all treatment cycles, the end-of-treatment response assessment was based on scans performed 6-8 weeks after the final dose of study treatment in both treatment arms.

Patients who discontinued study treatment early for a reason other than disease progression had a response assessment 6-8 weeks from the final dose of study treatment, unless the most recent tumor assessment was +28 days of the final dose of study treatment.

In the follow-up period, diagnostic CT scans with or without PET/CT scans was obtained until patients develop progressive disease, including patients who proceed to new anti-lymphoma therapy, as follows:
Patients in the Glofit-GemOx arm had scans at 3 and 6 months after the end-of-treatment response assessment, then every 6 months until disease progression, or until 2 years from the end-of-treatment response assessment, whichever occurs earlier.
Patients in the R-GemOx arm had scans at 3, 6, and 9 months after the end-of-treatment response assessment, then every 6 months until disease progression, or until 2 years from the end-of-treatment response assessment, whichever occurs earlier.

PET/CT scans should be obtained in the follow-up period at any time clinical findings suggest disease progression, and response should be recorded on the eCRF. Imaging with CT is currently the preferred method for measuring target lesions selected for response assessment for NHL (Cheson et al. 2014), though MRI scans are acceptable in this study if CT scans are contraindicated; conventional CT and MRI scans should be performed with contiguous cuts of ≤8 mm in slice thickness. CT scans (with IV contrast) should include the chest, abdomen, and pelvis; CT scans of the neck should be included if clinically indicated or if involved at baseline. If disease in other areas is suspected, additional areas should be imaged. CT scans for response assessment may be limited to areas of prior involvement only if required by local regulatory authorities.

In patients for whom contrast is contraindicated (e.g., patients with contrast allergy or impaired renal clearance), CT or combined PET/CT scans without contrast, or MRI scans are permitted so long as they permit consistent and precise measurement of target lesions during the study treatment period.

At all times during the study, diagnosis of disease progression based on clinical examination must be confirmed on PET/CT and/or CT scan (or MRI scan if CT scan is contraindicated) as soon as feasible and prior to initiation of a new anti-lymphoma therapy.

All imaging data for tumor assessments were and will be collected by the sponsor to enable centralized independent review of response endpoints by an IRC. However, clinical decisions during the study were based on investigator-based tumor assessments.

### Statistical Methods

This was a Phase III, multicenter, open-label, randomized controlled trial designed to evaluate efficacy and safety of glofitamab in combination with gemcitabine plus oxaliplatin in patients with R/R DLBCL.

The analysis populations were defined as follows:
Intent-to-treat (ITT) population: all randomized patients.
Safety-evaluable population: patients who received any amount of any study treatment.
Patient reported outcome (PRO)-evaluable population: all randomized patients who had a baseline and at least one postbaseline assessment. PRO-evaluable population were used for descriptive analyses of visit summary and change from baseline analyses. All randomized patients (ITT) were used for completion analyses and time-to deterioration analyses.
PK-evaluable population: all patients who received at least one dose of study treatment in the Glofit-GemOx arm and had at least one postdose concentration result.
Immunogenicity population: all patients who had at least one predose and one postdose ADA assessment.

For all efficacy analyses (including PROs), patients were grouped according to the treatment assigned at randomization. For all safety analyses, patients were grouped according to the treatment actually received (patients with any dose of glofitamab or obinutuzumab were analyzed in the Glofit-GemOx arm).

Hypothesis tests were two-sided, unless otherwise indicated. The type I error (a) for this study is 0.05 (two-sided).

### Primary Analysis

The primary objective of this study was to evaluate the efficacy of Glofit-GemOx relative to R-GemOx in patients with R/R DLBCL as measured by OS, defined as the time from randomization to death from any cause.

The primary efficacy analysis was completed on the intent-to-treat population, with patients grouped according to their treatment assigned at randomization.

For patients who have not died at the clinical cutoff date for analysis, OS was censored on the last date when the patients are known to be alive. Patients who did not have information after baseline were censored at the date of randomization.

The primary analysis of the study tested the equality of OS distribution in Glofit-GemOx versus R-GemOx:

$${H}_{0} : {OS}_{GLOFIT - GEMOX =} O {S}_{R - GEMOX} VERSUS {H}_{1} : {OS}_{GLOFIT - GEMOX} \neq {OS}_{R - GEMOX}$$

Treatment comparison was made using a two-sided level 0.05 stratified log-rank test. The following randomization stratification factors were used in the efficacy analyses: number of previous lines of systemic therapy for DLBCL (1 vs. ≥2) and outcome of last systemic therapy (relapsed vs. refractory). The Kaplan-Meier method was used to estimate the median OS, if reached, and OS distribution for each treatment arm. The Brookmeyer-Crowley methodology (Brookmeyer and Crowley 1982) was used to construct the 95% CI for the median OS for each treatment arm. Cox proportional-hazards models were used to estimate the stratified hazard ratio and its 95% CI.

### Secondary Efficacy Endpoints

To control the overall type I error rate at a two-sided 0.05 level of significance, a hierarchical testing procedure was used to adjust for multiple statistical testing of the primary and key secondary efficacy endpoints. The following key secondary endpoints were formally tested:
PFS by IRC.
Best overall CR rate (based on response including PET/CT data) by IRC.
Best ORR (based on response including PET/CT data) by IRC

PFS was defined as the time from randomization to the first occurrence of disease progression, or death due to any cause, whichever occurs first. Disease progression was determined by the IRC and also by the investigator. Patients who had neither progressed nor died at the time of analysis (CCOD) and patients who were lost to follow-up were censored on the date of the last evaluable tumor assessment. Patients who did not undergo a postbaseline tumor assessment were censored at the time of randomization. The methodologies detailed for the OS analysis were used for the PFS analysis.

The CR rate was defined as the proportion of patients whose best overall response was a CR on PET/CT during the study, according to the 2014 Lugano Response Criteria, as determined by the IRC and the investigator.

The ORR was defined as the proportion of patients whose best overall response was a PR or a CR during the study, according to the 2014 Lugano Response Criteria, as determined by the IRC and the investigator.

For the secondary efficacy endpoints of CR rate and ORR, an estimate of CR rate or ORR and its 95% CI was calculated using the Clopper-Pearson method for each treatment arm. CR rate and ORR were compared between treatment arms using the Cochran-Mantel-Haenszel test stratified by the randomization stratification factors.

Responses after initiation of new anti-lymphoma therapy were included in the analysis of CR rate and ORR.

Duration of objective response was defined as the time interval from the date of the first occurrence of an objective response (PR or CR) until the first date that progressive disease or death was documented, whichever occurred first.

Duration of CR was defined as the time interval from the date of the first occurrence of CR until the first date that progressive disease or death was documented, whichever occurs first. Patients who had not progressed and who had not died at the time of analysis were censored at the time of last tumor assessment date. If no tumor assessments were performed after the date of the first occurrence of a response, duration of response were censored at the date of the first occurrence of a response. The methodologies detailed for the OS analysis were used for the duration of response analysis, except that the analysis was not stratified.

For the PRO questionnaires (EORTC QLQ-C30 and FACT-Lym LymS), visit summary and change from baseline analyses, and mixed-effects repeated measures modeling at each timepoint was performed by treatment arm. In addition, time to deterioration analyses were performed on the EORTC QLQ-C30 and FACT-Lym LymS. For the EORTC QLQ-C30, time to deterioration in physical functioning and/or fatigue was defined as the time from randomization to the first documentation of a 10-point or more decrease. For the FACT-Lym LymS, time to deterioration in lymphoma-specific symptoms was defined as the time from randomization to the first documentation of a 3-point or more decrease. A completion analysis was performed for each overall questionnaire at each timepoint. Completion rates were summarized by number and proportion of patients among those expected to complete the questionnaire at each timepoint.

FACT-Lym LymS and EORTC QLQ-C30 questionnaire analysis was completed on the PRO-evaluable population. The percentage of patients randomized to each arm who completed the questionnaire at each assessment point after baseline (PRO completion rates) were calculated and compared based on the ITT population.

### Safety Analyses

All safety analyses were based on the safety evaluable population, defined as all randomized patients who received any amount of any study treatment, grouped according to treatment received. Safety was assessed through summaries of exposure to study treatment, adverse events, changes in laboratory test results, and changes in vital signs and ECGs.

Study treatment exposure (such as treatment duration, total dose received, and number of cycles and dose modifications) was summarized with descriptive statistics.

All verbatim adverse event terms were mapped to Medical Dictionary for Regulatory Activities (MedDRA) thesaurus terms. Other than adverse events of CRS, which were graded according to ASTCT CRS Consensus Grading, the adverse event severity grading scale for the NCI CTCAE v5.0 was used for assessing adverse event severity. All adverse events, serious adverse events, adverse events leading to death, adverse events of special interest, and adverse events leading to study treatment discontinuation that occurred on or after the first dose of study treatment (i.e., treatment emergent adverse events) are summarized by mapped term, appropriate thesaurus level, and severity grade. For events of varying severity, the highest grade are used in the summaries. Deaths and cause of death are summarized.

Relevant laboratory, vital sign (pulse rate, respiratory rate, blood pressure, pulse oximetry, and temperature), and ECG data is displayed by time, with grades identified where appropriate. Additionally, a shift table of selected laboratory tests is used to summarize the baseline and maximum postbaseline severity grade. Changes in vital signs and ECGs are summarized.

Exploratory Safety Analyses for Patients Who Receive HSCT or CAR T-Cell Therapy after Study Treatment

A summary of 100-day non-relapse mortality is generated for patients who received treatment with HSCT after study treatment. The following data is summarized for patients who receive CAR T-cell therapy after study treatment: 100-day non-relapse mortality, incidence and severity of CRS, and incidence and severity of ICANS.

### Discussion

The R-GemOx regimen is widely utilized for patients with DLBCL in second-line or subsequent therapy in patients who are not candidates for ASCT or relapsed after ASCT. The use of R-GemOx in relapsed/refractory DLBCL is based on several Phase II studies (El Gnaoui et al. Ann Oncol. 18 (8): 1363-1368, 2007; Lopez et al. Eur J Haematol. 80 (2): 127-132, 2008; Corazzelli et al. Cancer Chemother Pharmacol. (64 (5): 907-916, 2009), the largest of which was a Phase II multicenter study enrolling 49 patients (Mounier et al. Haematologica. 98 (11): 1726-1731, 2013). Studies in patients with one or more prior lines of therapy reported findings as follows:
In El Gnaoui et al., 46 patients (33 patients with DLBCL) were treated with rituximab 375 mg/m², gemcitabine 1000 mg/m², and oxaliplatin 100 mg/m² every 14 days. Median duration of exposure was 16 weeks. CR rate was 73% in patients with DLBCL. Median time to progression in DLBCL patients was 24 months, and 2-year event-free survival in this population was 42% (El Gnaoui et al. 2007). Median OS for DLBCL was not reported.
In Lopez et al., 35 patients were treated with rituximab 375 mg/m², gemcitabine 1000 mg/m², and oxaliplatin 100 mg/m² every 2-3 weeks; patients treated later in the study were treated every 3 weeks. Patients were treated for a median of 4 cycles (range 2-8 cycles). CR rate was 34% and median OS was 9.1 months. The study noted that its population was older than that treated in the El Gnaoui study and had higher risk features (Lopez et al., 2008).
In Corazzelli et al., 32 patients (16 with DLBCL) were treated with rituximab 375 mg/m², gemcitabine 1200 mg/m², and oxaliplatin 120 mg/mg² every 2 weeks.
The majority of patients received complete therapy (6 cycles). CR rate in DLBCL was 56%; survival was not reported specific to DLBCL (Corazzelli et al. 2009).
In Mounier et al., 49 patients with DLBCL were treated as in the El Gnaoui study. The median age of patients was 69 years, 88% of patients had stage III/IV disease, 35% of patients had received prior ASCT, and 74% of patients were in first relapse (other patients had primary refractory disease or were in second relapse). Low rates of renal toxicity (1 patient) and febrile neutropenia (4% of cycles) were reported. At the primary analysis timepoint, an ORR of 61% and CR rate of 44% was achieved. Median PFS was 5 months and median OS was 11 months. The most common toxicities during treatment were hematologic with 98% of patients, including Grade 3 thrombocytopenia in 44% of patients, Grade 4 neutropenia in 42% of cycles, and 33% receiving at least one red blood cell transfusion. A dose reduction of oxaliplatin was required in 45% who had at least Grade 2 neuropathy. Only two patients discontinued treatment due to toxicity; one treatment related death, thrombotic microangiopathy attributed to gemcitabine, was reported (Mounier et al. 2013).

Alternatively, BR has been widely evaluated as a regimen in this population. One study conducted by Vacirca and colleagues included 95% of patients who had previously received rituximab therapy and the CR rate with BR given as second-line treatment and subsequent therapy was 15.3%, with a median PFS of 3.6 months. OS was not reported due to a high rate of censoring (Vacirca et al. Ann Hematol. 93 (3): 403-409, 2014). A higher response rate was reported for the BR combination in Japanese patients with relapsed (but not refractory) DLBCL in a study published by Ohmachi and colleagues, with a CR of 37% and median PFS of 6.7 months (Ohmachi et al. J Clin Oncol. 31 (17): 2103-2109, 2013).

Polatuzumab vedotin, in combination with bendamustine and a rituximab product, was recently granted accelerated approval in multiple regions for the treatment of adult patients with R/R DLBCL, not otherwise specified (NOS). In a study conducted by Sehn et al. (Sehn et al. J Clin Oncol. 38 (2): 155-165, 2020), patients with R/R DLBCL who were not candidates for transplant were randomized to receive either polatuzumab vedotin in combination with BR (pola-BR) or BR, with 40 patients in each arm. Patients in the pola-BR arm had a 40% CR rate on positron emission tomography (PET) imaging at the end of treatment, with a median PFS (assessed by an independent review committee) of 9.5 months and a median OS of 12.4 months. Patients in the BR arm had an 18% CR rate on PET imaging at the end of treatment, with a median PFS of 3.7 months and a median OS of 4.7 months (Sehn et al. 2020; Polivy® U.S. Package Insert).

It is difficult to compare directly the cohorts reported in these studies, although clearly the outcomes of patients who are not candidates for transplant are very poor. There are differences between the design of these studies based on eligibility criteria (i.e., limitations on prior lines of therapy, refractoriness) as well as historical context (e.g., how many patients had prior exposure to rituximab or the type of first-line therapy).

T-cells re-engineered to express a chimeric antigen receptor targeting a relevant tumor target, or CAR T-cells, have recently produced high rates of durable responses for patients with R/R DLBCL after two or more lines of systemic therapy (Schuster et al. N Engl J Med. 380 (1): 45-56, 2019). Although the data for CAR T-cell therapies after two or more lines of systemic therapy are promising (Neelapu et al. N Engl J Med. 377 (26): 2531-2544, 2017; Schuster et al. 2019), ensuring the consistency, quality, and dose of autologous cell-based therapies remains non-trivial and potentially limits the broad adoption of this therapeutic mode beyond specialized centers. In addition, the cellular processing time may be prohibitive for a substantial proportion of patients with rapidly progressive disease who may not have time to wait for leukapheresis scheduling or manufacturing time for CAR T-cell therapy.

The limited accessibility of CAR T-cell therapies to the broader patient population, coupled with the challenging toxicity profile, demonstrates that a high, unmet medical need continues to exist. Novel therapies are needed to further improve the treatment outcomes of patients with R/R DLBCL and to offer more patients a more effective treatment with improved survival.

Combining glofitamab with gemcitabine plus oxaliplatin provides improved response rates, and improved OS, compared with the combination of rituximab and GemOx, while maintaining an acceptable safety profile. The strong efficacy benefits patients with DLBCL in the second-line and subsequent settings with a favorable benefit-risk profile. Abbreviations used in the Examples described herein are shown below in Table 8.

TABLE 8
Abbreviations

| Abbreviation | Definition |
|---|---|
| ADA | anti-drug antibody |
| ASCT | autologous stem cell transplant |
| ASTCT | American Society for Transplantation |
| | and Cell Therapy |
| AUC | area under the concentration-time |
| | curve |
| BR | bendamustine in combination with |
| | rituximab |
| CAR | chimeric antigen receptor |
| CCOD | clinical cutoff date |
| CDC | complement-dependent cytotoxicity |
| CHOP | cyclophosphamide, doxorubicin, |
| | vincristine, and prednisone |
| CNS | central nervous system |
| COVID-19 | coronavirus disease 2019 |
| CR | complete response |
| CrCl | creatinine clearance |
| CRS | cytokine release syndrome |
| CT | computed tomography |
| CTCAE | Common Terminology Criteria for |
| | Adverse Events |
| ctDNA | circulating-tumor DNA |
| DHAC | dexamethasone, cytarabine, |
| | carboplatin |
| DHAP | dexamethasone, cytarabine, cisplatin |
| DLBCL | diffuse large B-cell lymphoma |
| EBV | Epstein-Barr virus |
| EC | Ethics Committee |
| EC50 | 50% effective concentration |
| ECOG | Eastern Cooperative Oncology Group |
| eCRF | electronic Case Report Form |
| EDC | electronic data capture |
| EORTC QLQ-C30 | European Organisation for Research |
| | and Treatment of Cancer Quality of |
| | Life-Core 30 Questionnaire |
| EQ-5D-5L | EuroQol 5-Level 5-Dimension |
| | Questionnaire |
| ESMO | European Society for Medical |
| | Oncology |
| Fab | fragment antigen-binding |
| FACT-Lym LymS | Functional Assessment of Cancer |
| | Therapy-Lymphoma Lymphoma |
| | subscale |
| Fc | fragment crystallizable |
| FcγR | Fc-γ receptor |
| FDA | (U.S.) Food and Drug Administration |
| FFPE | formalin-fixed, paraffin-embedded |
| FL | follicular lymphoma |
| G | obinutuzumab |
| GCB | germinal center B-cell like |
| G-CHOP | obinutuzumab in combination with |
| | cyclophosphamide, doxorubicin, |
| | vincristine, and prednisone |
| G-CSF | granulocyte colony-stimulating factor |
| GDP | gemcitabine, dexamethasone, |
| | cisplatin/carboplatin |
| GemOx | gemcitabine plus oxaliplatin |
| Glofit-GemOx | Glofitamab in combination with |
| | gemcitabine and oxaliplatin |
| Granzyme B | cytotoxic granule release |
| HBcAb | hepatitis B core antibody |
| HBsAg | hepatitis B surface antigen |
| HBV | hepatitis B virus |
| HCV | hepatitis C virus |
| HLH | hemophagocytic lymphohistiocytosis |
| HRQoL | health-related quality of life |
| HSCT | hematopoietic stem cell transplant |
| ICANS | immune effector cell-associated |
| | neurotoxicity syndrome |
| ICE | ifosfamide, carboplatin, etoposide |
| iDCC | independent Data Coordinating Center |
| iDMC | independent Data Monitoring |
| | Committee |
| IFN(-γ) | interferon(-γ) |
| IHC | immunohistochemistry |
| IL-2 (-6, -10, -12) | interleukin-2 (-6, -10, -12) |
| IL-6R | interleukin-6 receptor |
| IMP | investigational medicinal product |
| IND | Investigational New Drug (Application) |
| IRB | Institutional Review Board |
| IRC | independent review committee |
| IRR | infusion-related reaction |
| ITT | intent to treat |
| IxRS | interactive voice or web-based |
| | response system |
| MRD | minimal residual disease |
| MRI | magnetic resonance imaging |
| NCCN | National Comprehensive Cancer |
| | Network |
| NCI | National Cancer Institute |
| NCI CTCAE v5.0 | National Cancer Institute Common |
| | Terminology Criteria for Adverse |
| | Events, Version 5.0 |
| NHL | non-Hodgkin lymphoma |
| NK | natural killer (cell) |
| NSG ™ | NOD scid gamma ™ |
| NOS | not otherwise specified |
| ORR | objective response rate |
| OS | overall survival |
| PBMC | peripheral blood mononuclear cell |
| PCR | polymerase chain reaction |
| PD | pharmacodynamic |
| PET | positron emission tomography |
| PFS | progression-free survival |
| PK | pharmacokinetic |
| PML | progressive multifocal |
| | leukoencephalopathy |
| pola-BR | polatuzumab vedotin in combination |
| | with bendamustine and rituximab |
| PR | partial response |
| PRO | patient-reported outcome |
| PVC | polyvinyl chloride |
| RBR | Research Biosample Repository |
| R-CHOP | rituximab in combination with |
| | cyclophosphamide, doxorubicin, |
| | vincristine, and prednisone |
| R-GemOx | rituximab in combination with |
| | gemcitabine and oxaliplatin |
| R/R | relapsed or refractory |
| SARS-CoV-2 | severe acute respiratory syndrome |
| | coronavirus 2 |
| TCR | T cell-receptor |
| TLS | tumor lysis syndrome |
| TNF-α | tumor necrosis factor-α |
| ULN | upper limit of normal |
| U.S. | United States |
| WES | whole exome sequencing |

### Statistical Analyses

### Primary Analysis

The primary objective of this study is to evaluate the efficacy of Glofit-GemOx relative to R-GemOx in patients with R/R DLBCL as measured by OS, defined as the time from randomization to death from any cause.

The primary efficacy analysis was completed on the intent-to-treat population, with patients grouped according to their treatment assigned at randomization.

For patients who have not died at the clinical cutoff date for analysis, OS was censored on the last date when the patients are known to be alive. Patients who do not have information after baseline were censored at the date of randomization.

The primary analysis of the study tested the equality of OS distribution in Glofit-GemOx versus R-GemOx:

$${H}_{0} : {OS}_{GLOFIT - GEMOX} = O {S}_{R - GEMOX} versus {H}_{1} : {OS}_{GLOFIT - GEM} \neq {OS}_{R - GEMOX}$$

Treatment comparison was made using a two-sided level 0.05 stratified log-rank test. The randomization stratification factors to be used in the efficacy analyses are number of previous lines of systemic therapy for DLBCL (1 vs. ≥2) and outcome of last systemic therapy (relapsed vs. refractory). The Kaplan-Meier method was used to estimate the median OS, if reached, and OS distribution for each treatment arm. The Brookmeyer-Crowley methodology (Brookmeyer and Crowley 1982) was used to construct the 95% CI for the median OS for each treatment arm. Cox proportional-hazards models were used to estimate the stratified hazard ratio and its 95% CI.

Further details, including intercurrent events, sensitivity analyses, evaluation of the proportional-hazards assumption and competing risks in the analyses of time-to-event endpoints, were provided in the statistical analysis plan.

### Example 2. Initial Results for a Phase III, Open-Label, Multicenter, Randomized Study Evaluating the Efficacy and Safety of Glofitamab in Combination with Gemcitabine Plus Oxaliplatin Versus Rituximab in Combination with Gemcitabine and Oxaliplatin in Patients with Relapsed/Refractory Diffuse Large B-Cell Lymphoma

Results are described for data cut-off as of March 2023. The Phase III Study described herein in Example 1 met its primary endpoint of a statistically significant and clinically meaningful overall survival in patients with R/R DLBCL who have failed one line of therapy and are not candidates for transplant, as well as patients who have failed at least two lines of therapy.

Efficacy results are summarized below in Table 9.

TABLE 9
Primary Efficacy Results

Overall Survival (OS, Primary Endpoint)
HR = 0.59 95% CI [0.40, 0.89], P value = 0.010706,
median OS: 9 months (R-GemOx) vs NE (non-evaluable; Glofit-GemOx,
18-mo OS rate: 54.6%)
Progression free survival (PFS) by IRC (independent review committee)
HR = 0.34, 95% CI [0.23, 0.49], P value < 0.00001,
median PFS: 2.6 months (R-GemOx) vs 10.4 months (Glofit-GemOx)
Best complete response (CR) rate by IRC
Best CRR (CR rate): 22% (R-GemOx) vs 50.3% (Glofit-GemOx),
P value < 0.0001
Duration of complete response (DOCR) by IRC
HR = 0.40, 95% CI [0.15, 1.06], P value = 0.0572,
median DOCR: 6.5 months (R-GemOx) vs 14.4 months (Glofit-GemOx)

The safety profile of Glofit-GemOx was consistent with the known risk of individual study drugs (e.g., glofitamab, gemcitabine, and oxaliplatin). The safety and toxicity profiles of Glofit-GemOx was manageable.

CRS was the most common adverse event, and mostly low grade, which was consistent with previous studies.

Detailed results for OS for ITT (intent to treat) patients are shown in FIG. 3 and in Table 10 below.

TABLE 10
OS - ITT population

| | R-GemOx | | Glofit-GemOx | |
|---|---|---|---|---|
| | (N = 91) | | (N = 183) | |
| No. of Events (%) | 40 | (44%) | 61 | (33.3%) |

Stratified Analysis ^{a}

| P-value (Log-rank) | 0.010706 | | | |
|---|---|---|---|---|
| Hazard Ratio (95% CI) | 0.59 (0.40, 0.89) | | | |
| Median (95% CI) | 9 | (7.3, 14.4) | NE | (13.8, NE) |
| 12 months OS | 44.6% | (31.6, 57.6) | 62.5% | (54.4, 70.7) |
| rate ^{b} (95% CI) | | | | |
| 18 months OS | 35.0% | (21.0, 49.0) | 54.6% | (44.7, 64.6) |
| rate ^{b} (95% CI) | | | | |

^{a} Stratification factors (IxRS (interactive voice or web-based response system)) are number of previous lines of systemic therapy for DLBCL (1 vs. ≥2) and outcome of last systemic therapy (relapsed vs. refractory)
^{b} Kaplan-Meier estimate

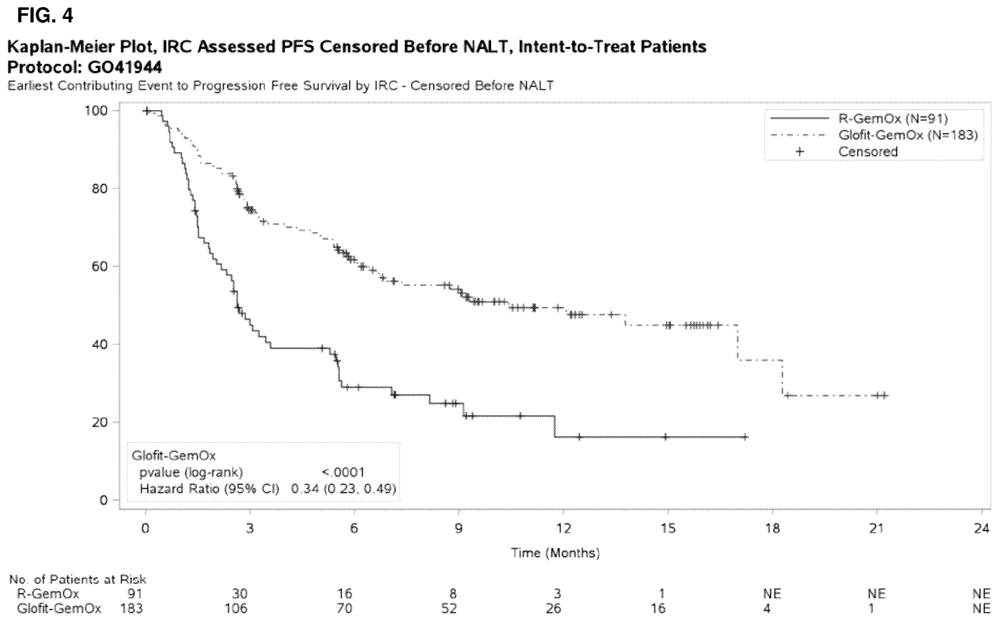

Detailed results for PFS for ITT (intent to treat) patients are shown in FIG. 4 and in Table 11 below.

TABLE 11
PFS - ITT population

| | R-GemOx | | Glofit-GemOx | |
|---|---|---|---|---|
| | (N = 91) | | (N = 183) | |
| No. of Events (%) | 54 | (59.3%) | 72 | (39.3%) |

Earliest Contributing Event

| | | |
|---|---|---|
| Death | 4 | 18 |
| Disease Progression | 50 | 54 |

Stratified Analysis ^{a}

| P-value (Log-rank) | <0.000001 | | | |
|---|---|---|---|---|
| Hazard Ratio (95% CI) | 0.34 (0.23, 0.49) | | | |
| Median (95% CI) | 2.6 | (2.0, 5.3) | 10.4 | (6.6, 18.3) |
| 6 months PFS | 29.0% | (18.1, 39.9) | 61.7% | (53.8, 69.7) |
| rate ^{b} (95% CI) | | | | |
| 12 months PFS | 16.3% | (3.9, 28.7) | 49.5% | (40.5, 58.5) |
| rate ^{b} (95% CI) | | | | |
| PFS follow up median | 7.2 | (0-17) | 9.2 | (0-21) |

(range, month)

^{a} Stratification factors (IxRS) are number of previous lines of systemic therapy for DLBCL (1 vs. ≥2) and outcome of last systemic therapy (relapsed vs. refractory)
^{b} Kaplan-Meier estimate

### Example 3. Updated Results for a Phase III, Open-Label, Multicenter, Randomized Study Evaluating the Efficacy and Safety of Glofitamab in Combination with Gemcitabine Plus Oxaliplatin Versus Rituximab in Combination with Gemcitabine and Oxaliplatin in Patients with Relapsed/Refractory Diffuse Large B-Cell Lymphoma

Results are described for data cut-off as of February 2024. The Phase III Study described herein in Example 1 continued to meet the primary endpoint of a statistically significant and clinically meaningful overall survival in patients with R/R DLBCL who have failed one line of therapy and are not candidates for transplant, as well as patients who have failed at least two lines of therapy.

### Efficacy Results

With additional 11 months follow-up (e.g., compared to primary analysis described in Example 2), the Phase III Study continues to demonstrate compelling survival benefit in Glofit-GemOx arm with further improvement in reducing the likelihood of death. In particular, median OS almost doubled for patients treated with Glofit-GemOx in comparison to control patients treated with R-GemOx. Best CR rate more than doubled for patients treated with Glofit-GemOx in comparison to control patients treated with R-GemOx.

Efficacy results are summarized below in Table 12.

TABLE 12
Updated Efficacy Results

Overall Survival (OS, Primary Endpoint)
HR = 0.62 95% CI [0.43, 0.88], P value = 0.006366
median OS: 12.9 months (R-GemOx) vs 25.5 (Glofit-GemOx)
Progression free survival (PFS) by IRC (independent review committee)
HR = 0.42, 95% CI [0.29, 0.61], P value = 0.000003
median PFS: 5.5 months (R-GemOx) vs 14.4 months (Glofit-GemOx)
Best complete response (CR) rate by IRC
Best CRR (CR rate): 26.4% (R-GemOx) vs 58.5% (Glofit-GemOx),
P value < 0.0001

The safety profile of Glofit-GemOx was consistent with the known risk of individual study drugs (e.g., glofitamab, gemcitabine, and oxaliplatin). The safety and toxicity profiles of Glofit-GemOx were manageable.

CRS was the most common adverse event, and mostly low grade, which was consistent with previous studies.

Continued OS benefit was observed, with HR=0.62 (p-value=0.0064). Detailed results for OS for ITT (intent to treat) patients are shown in Table 13 below.

TABLE 13
OS - ITT population

| | R-GemOx | | Glofit-GemOx | |
|---|---|---|---|---|
| | (N = 91) | | (N = 183) | |
| No. of Events (%) | 52 | (57.1%) | 80 | (43.7%) |

Stratified Analysis ^{a}

| P-value (Log-rank) | 0.006366 | | | |
|---|---|---|---|---|
| Hazard Ratio (95% CI) | 0.62 (0.43, 0.88) | | | |
| Median (95% CI) | 12.9 | (7.9, 18.5) | 25.5 | (18.3, NE) |
| 12 months OS | 52.5% | (41.6, 63.3) | 62.9% | (55.7, 70.1) |
| rate ^{b} (95% CI) | | | | |
| 18 months OS | 39.7% | (28.7, 50.7) | 57.6% | (50.2, 65.1) |
| rate ^{b} (95% CI) | | | | |
| 24 months OS | 33.5% | (22.2, 44.9) | 52.8% | (44.8, 60.7) |
| rate ^{b} (95% CI) | | | | |

^{a} Stratification factors (IxRS (interactive voice or web-based response system)) are number of previous lines of systemic therapy for DLBCL (1 vs. ≥2) and outcome of last systemic therapy (relapsed vs. refractory)
^{b} Kaplan-Meier estimate

Primary endpoint for OS maintained with HR=0.62 and median OS doubled compared to control arm (25.5 months vs 12.9 months).

Continued PFS benefit was observed with HR=0.42 (p-value=0.000003). Detailed results for PFS for ITT (intent to treat) patients are in Table 14 below.

TABLE 14
PFS - ITT population

| | R-GemOx | | Glofit-GemOx | |
|---|---|---|---|---|
| | (N = 91) | | (N = 183) | |
| No. of Events (%) | 47 | (51.6%) | 86 | (47%) |

Earliest Contributing Event

| | | |
|---|---|---|
| Death | 7 | 34 |
| Disease Progression | 40 | 52 |

Stratified Analysis ^{a}

| P-value (Log-rank) | 0.000003 | | | |
|---|---|---|---|---|
| Hazard Ratio (95% CI) | 0.42 (0.29, 0.61) | | | |
| Median (95% CI) | 5.5 | | 14.4 | |
| 6 months PFS | 42.9% | (30.9, 54.9) | 67.7% | (60.6, 74.8) |
| rate ^{b} (95% CI) | | | | |
| 12 months PFS | 29.6% | (16.6, 42.5) | 53.1% | (45.3, 60.9) |
| rate ^{b} (95% CI) | | | | |
| PFS follow up median | 7.2 | (0-27) | 16.2 | (0-33) |

(range, month)

^{a} Stratification factors (IxRS) are number of previous lines of systemic therapy for DLBCL (1 vs. ≥2) and outcome of last systemic therapy (relapsed vs. refractory)
^{b} Kaplan-Meier estimate

Primary endpoint for PFS maintained with HR=0.42. Median PFS for patients treated with Glofit-GemOx was more than 2.5-fold that of patients treated with R-GemOx (14.4 months vs 5.5 months)

A clinically meaningful and higher improvement of CR rate was observed in comparison to primary analysis described in Example 2. Highly durable CR was observed with Glofit-GemOx treatment. Limited CRs in control group (R-GemOx; n=24) impacts interpretation of data. Detailed results for CR for ITT (intent to treat) patients are in Table 15 below.

TABLE 15
CR - ITT population

| | R-GemOx | Glofit-GemOx |
|---|---|---|
| | (N = 91) | (N = 183) |
| Complete Responders by IRC (%) | 24 (26.4%) | 107 (58.5%) |
| (95% CI)^{b} | (17.7%, 36.7%) | (51.0%, 65.7%) |
| Difference in Response Rate | 32.1% (19.7, 44.5) | |
| (95% CI)^{c} | | |
| Descriptive P-value^{d} | <0.0001 | |
| Best ORR by IRC^{e} | 38 (41.8%) | 126 (68.9%) |
| (95% CI)^{b} | (31.5%, 52.6%) | (61.6%, 75.5%) |

^{a} Stratification factors (IxRS) are number of previous lines of systemic therapy for DLBCL (1 vs. ≥2) and outcome of last systemic therapy (relapsed vs. refractory)
^{b}95% CI for response rate are constructed using the Clopper-Pearson method.
^{c}95% CI for difference in response rates are constructed using Wald method.
^{d}Based on Cochran-Mantel-Haenszel method.
^{e}added for reference only as ORR is not formally tested in hierarchical order and without alpha control.

Further improved difference in CR rates between treatment vs control arms was observed (58.5% for Glofit-GemOx; 26.4% for R-GemOx).

### Safety Results

The safety profile of Glofit-GemOx was tolerable and consistent with the known risk of individual study drugs. Rates of serious adverse events (SAEs, Grade 3+adverse events (AEs), and AEs leading to discontinuation are higher with Glofit-GemOx compared to R-GemOx, likely due to higher exposure in Glofit-GemOx. Exposure-adjusted AE rates were broadly similar between Glofit-GemOx and R-GemOx arms with the exception of higher SAEs in Glofit-GemOx patients, predominantly due to cytokine release syndrome (CRS) and gastrointestinal (GI) disorders. CRS was the most commonly-observed AE. Most CRS events were low grade, consistent with previous studies. Any grade CRS: 44.2% (Gr1: 31.4%; Gr2: 10.5%; Gr3 *: 2.3%; Gr4+: 0%). *: one patient experienced a grade 3 CRS event confounded by a concurrent Grade 5 septic shock that required multiple pressors—this event may be viewed as a Grade 4 of 5 CRS event by health authorities. Results are detailed in Tables 16 and 17 below.

TABLE 16
Safety data

| | | | Glofit-GemOx | | Glofit-GemOx | |
|---|---|---|---|---|---|---|
| | R-GemOx | | (Glofit Exposed) | | (Any Exposed) | |
| | (N = 88) | | (N = 172) | | (N = 180) | |
| Number of infusions | 4 | (1-8) | 12 | (1-14) | 12 | (1-14) |
| Median (range) | | | | | | |
| SAE, n (%) | 15 | (17.0%) | 90 | (52.3%) | 98 | (54.4%) |
| Grade 3+ AEs, n (%) | 36 | (40.9%) | 132 | (76.7%) | 140 | (77.8%) |
| Grade 5 AEs, n (%) | 4 | (4.5%) | 12 | (7.0%) | 15 | (8.3%) |
| AE leading to | 11 | (12.5%) | 43 | (25.0%) | 48 | (26.7%) |
| any treatment | | | | | | |
| discontinuation, n (%) | | | | | | |

TABLE 17
Safety data - exposure adjusted

| | | Glofit-GemOx | Glofit-GemOx |
|---|---|---|---|
| AE rate per 100 | R-GemOx | (Glofit Exposed) | (Any Exposed) |
| patient-years* | (N = 88) | (N = 172) | (N = 180) |
| Total patient-years at risk | 18.0 | 81.3 | 81.4 |
| SAE, n | 83.3 | 110.7** | 120.4** |
| Grade 3+ AEs, n | 199.9 | 162.3 | 172 |
| Grade 5 AEs, n | 22.2 | 14.8 | 18.4 |
| COVID-19 | 44.4 | 36.9 | 40.6 |
| Discontinuations | 61.1 | 52.9 | 59.0 |

*Exposure-adjusted AE (AE rate per 100 patient-years) is calculated based on a patient duration on all study treatment from first to last dose.
**Driven by CRS and events from the GI disorders system order class (SOC).

Exposure adjusted AE rates were broadly similar between R-GemOx (control) and Glofit-GemOx (treatment) groups with the exception of SAEs, in which the difference was predominantly caused by CRS and GI disorders.

Comparator data from Cazelles et al. Leuk Lymph. 62 (9): 2161-2168, 2021 (“Cazelles”), which studied R-GemOx, and Held et al. oral presentation at ASH Annual Meeting & Exposition, 2023 (“NIVEAU”), which examined nivolumab+R-GemOx in comparison to R-GemOx alone, are shown below in Table 18.

TABLE 18
Comparator data from Cazelles and NIVEAU

| | Cazelles (N = 108) | NIVEAU (N = 88) |
|---|---|---|
| Median age | 72 (24-89) | 76 |
| Primary Refractory | NR | 37% |
| 2 L population | 58% | 100% |
| 1-year OS | NR | 48% |
| 1-year PFS | NR | 28 |
| Median OS | 8 months | NR |
| Median PFS | 3 months | NR |
| CR Rate | 26% | 20% |

### Example 4. Glofitamab Plus Gemcitabine and Oxaliplatin (Glofit-GemOx) for Relapsed/Refractory (R/R) Diffuse Large B-Cell Lymphoma (DLBCL): Results of a Global Randomized Phase III Trial (STARGLO)

Updated results are described for the Phase III Study described herein in Example 1. In particular, efficacy and safety results of Glofit-GemOx (glofitamab, gemcitabine, and oxaliplatin) versus rituximab (R)-GemOx in patients with relapsed/refractory (R/R) diffuse large B cell lymphoma (DLBCL) after >1 prior line of therapy from the global, randomized, Phase III STARGLO trial (NCT04408638) are described.

### Methods:

Patients were randomized 2:1 to receive either Glofit-GemOx (8 cycles, plus 4 cycles glofitamab monotherapy) or R-GemOx (8 cycles) and were stratified by number of prior therapies (1 vs ≥2) and refractoriness to last treatment. Following obinutuzumab pretreatment, glofitamab was administered during Cycle 1 as weekly step-up doses (2.5/10 mg), then the 30 mg target dose every 21 days from Cycle 2 Day 1. Patients enrolling after 1 prior therapy were considered ineligible for autologous stem cell transplant (ASCT) based on age ≥70 years, organ dysfunction, Eastern Cooperative Oncology Group (ECOG) performance status ≥2, patient refusal for ASCT, or other investigator-assessed comorbidities.

Primary endpoint was overall survival (OS). Secondary endpoints included independent review committee (IRC)-assessed progression-free survival (PFS) and complete remission (CR) rate.

### Results:

In total, 274 patients with DLBCL were enrolled (Glofit-GemOx, n=183; R-GemOx, n=91), including 172 (62.8%) after 1 prior therapy and 102 (37.2%) after ≥2 prior therapies. Overall, 153 (55.8%) patients had primary refractory disease and 166 (60.6%) were refractory to last therapy.

At primary analysis (cut-off date Mar. 29, 2023), there was a significant OS benefit with Glofit-GemOx versus R-GemOx (hazard ratio [HR] 0.59, 95% confidence interval [CI]: 0.40-0.89; p=0.011). With a median follow-up of 11.3 months, median OS was not reached (95% CI: 13.8-not evaluable) for Glofit-GemOx versus 9 months for R-GemOx (95% CI: 7.3-14.4). Significant benefit of Glofit-GemOx was also observed in IRC-assessed PFS (HR 0.37, 95% CI: 0.25-0.55; p<0.0001) and CR rate (50.3 vs 22.0%; 95% CI: 16.3-40.3, p<0.0001).

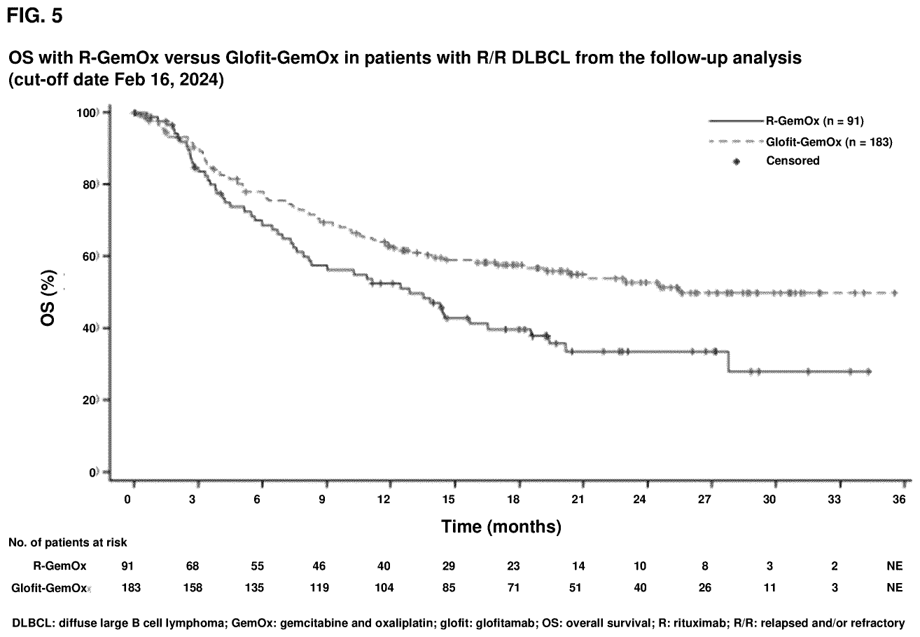

A follow-up analysis was conducted once all patients had completed therapy (cut-off date Feb. 16, 2024; median follow-up of 20.7 months). Glofit-GemOx continued to demonstrate superior median OS (25.5 vs 12.9 months; HR 0.62, 95% CI: 0.43-0.88; FIG. 5), median PFS (13.8 vs 3.6 months; HR 0.40, 95% CI: 0.28-0.57), and CR rate (58.5 vs 25.3%) versus R-GemOx.

Median number of cycles received was higher with Glofit-GemOx versus R-GemOx (11 vs 4). Adverse event (AE) rates were higher with Glofit-GemOx versus R-GemOx, including Grade (Gr) 3-4 AEs (69.4 vs 36.4%), Gr 5 AEs (8.3 vs 4.5%; primarily driven by an imbalance of COVID-19 AEs), and serious AEs (54.4 vs 17.0%; primarily CRS). Adjusting for exposure differences, AE rates were similar between arms. In patients exposed to glofitamab, CRS was the most frequently reported AE (Gr 1:31.4%, Gr 2:10.5%, and Gr 3:2.3%), and events consistent with immune effector cell-associated neurotoxicity syndrome were reported in four patients (2.3%), all of which were concurrent with CRS.

### Summary/Conclusion:

Glofit-GemOx demonstrated significant benefit in OS, PFS, and CR rate over R-GemOx in patients with R/R DLBCL who were ineligible for ASCT. Glofitamab is the first CD20: CD3 bispecific antibody to demonstrate survival benefit in DLBCL in a randomized Phase III trial. Glofit-GemOx is a fixed-duration, off-the-shelf regimen which was well tolerated, with a safety profile consistent with known risks of each individual agent.

### Example 5. In Vivo Study to Evaluate Effect of Dexamethasone Scheduling on CRS and Efficacy of Glofitamab in Combination with Gemcitabine and Oxaliplatin

Glofitamab, a T-cell bispecific antibody (TCB), has shown promise in cancer therapy however it can induce cytokine release syndrome (CRS), a significant side effect that requires management. To address this, the role of dexamethasone in mitigating CRS induced by glofitamab was evaluated in humanized mice.

Humanized mice were treated with various dosing regimens of dexamethasone, administered before and after glofitamab infusion. Serum cytokine levels were assessed at 4 and 28 hours posttreatment to assess the efficacy of dexamethasone in controlling CRS. Additionally, the impact of dexamethasone on the antitumor efficacy of glofitamab was also assessed.

Dexamethasone premedication significantly reduced cytokine levels early after glofitamab infusion, with additional doses providing further reduction at later time points. Posttreatment dexamethasone also effectively mitigated cytokine levels for an extended duration. Importantly, dexamethasone did not interfere with the antitumor activity of glofitamab.

These results indicated that dexamethasone is a viable option for managing CRS in patients treated with glofitamab, enhancing the safety profile of the therapy without diminishing its antitumor potential.

### Animals

BRGS-CD47 female mice (immunodeficient mice expressing human SIRPA and CD47 were developed by Roche, and breeding was outsourced to Jackson Laboratories; Sacramento, CA) were humanized at 3-4 weeks of age by total body irradiation using 200 cGy, followed by intravenous injection of 105 human hematopoietic stem cells. Twelve to fifteen weeks after the stem cell injection, humanized BRGS-CD47 mice were screened for human cell engraftment in the blood by flow cytometry. The humanization and screening process was conducted at the Jackson Laboratory, and only humanized BRGS-CD47 mice with a humanization rate greater than 20% (i.e., more than 20% of circulating human immune cells within all leukocytes) were shipped to Roche Innovation Center Zurich for subsequent experiments. Upon arrival, mice were maintained under specific-pathogen-free conditions and daily 12-hour light/dark cycles according to committed guidelines (GV-Solas, Felasa, TierschG). The experimental study protocol was reviewed and approved by the local government (License ZH180-20). Health monitoring was performed on a regular basis.

### Tumor Cell Culture and Tumor Cell Injection

For this study, human diffuse large B-cell lymphoma cells (WSU-DLCL2), originally obtained from the European Collection of Cell Culture (Salisbury, UK), were expanded and deposited in the Roche Glycart cell bank. Cells were cultured in Roswell Park Memorial Institute (RPMI) 1640 containing 10% fetal bovine serum (FBS; Catalog No. A4766801; Gibco, Thermo Fisher Scientific; Waltham, MA) and 1× GlutaMAX® (Catalog No. 35050061; Gibco). The cells were cultured at 37° C. in a water-saturated atmosphere containing 5% CO2. Cells were washed once with RPMI 1640 (no FBS) and resuspended in a 1:1 mixture of growth factor-reduced Matrigel®/RPMI 1640 (no FBS) at a final cell density of 15×106 cells/mL. The cells were injected subcutaneously (1.5×106 cells/animal in 100 μL) in the flank of mice (n=100).

### Treatment Injection

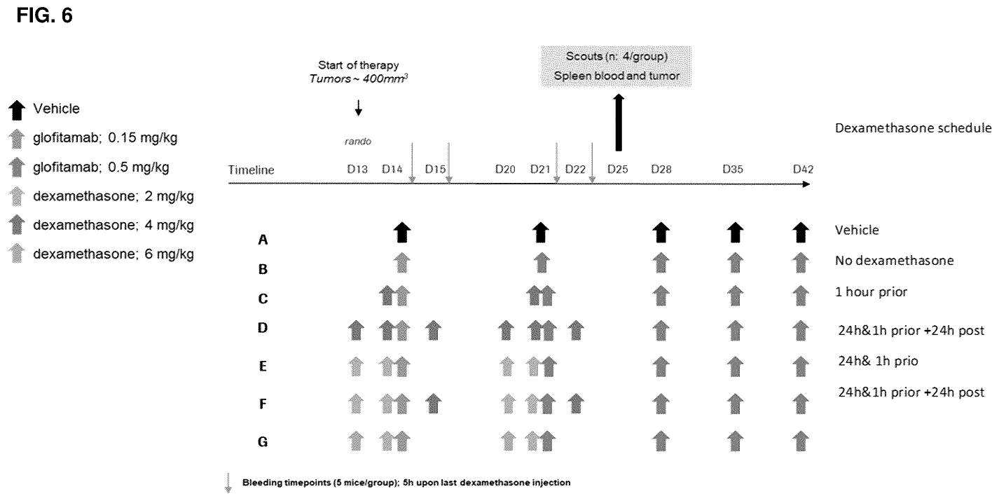

Treatment started on Day 13 after tumor cell injection when the average tumor volume reached approximately 400 mm³, Group A animals (n=14) were injected intravenously with histidine buffer (vehicle; 200 μL) once weekly for 5 weeks. Group B animals (n=14) were injected intravenously with glofitamab only (0.15 mg/kg at first and 0.5 mg/kg at subsequent injections in 200 μL of histidine buffer) once weekly for 5 weeks. The same administration scheme as in Group B was used for glofitamab in Groups C-G. To study the impact of different doses and schedules of dexamethasone treatment on cytokine release and tumor growth inhibition, mice in Groups C-G also received dexamethasone orally at different doses and schedule prior to and after the first and second glofitamab injection:
Group A: vehicle, no dexamethasone
Group B: glofitamab weekly, 0.15 mg/kg first injection, 0.5 mg/kg subsequent injections, no dexamethasone
Group C: glofitamab weekly, 0.15 mg/kg first injection, 0.5 mg/kg subsequent injections, dexamethasone 4 mg/kg 1 hour prior to first two glofitamab doses
Group D: glofitamab weekly, 0.15 mg/kg first injection, 0.5 mg/kg subsequent injections, dexamethasone 4 mg/kg 24 hours and 1 hour prior to first two glofitamab doses, and 24 hours post the first two glofitamab doses
Group E: glofitamab weekly, 0.15 mg/kg first injection, 0.5 mg/kg subsequent injections, dexamethasone 2 mg/kg 24 hours and 1 hour prior to first two glofitamab doses.
Group F: glofitamab weekly, 0.15 mg/kg first injection, 0.5 mg/kg subsequent injections, dexamethasone 2 mg/kg 24 hours and 1 hour prior to first two glofitamab doses and dexamethasone 4 mg/kg 24 hours post the first two glofitamab doses
Group G: glofitamab weekly, 0.15 mg/kg first injection, 0.5 mg/kg subsequent injections, dexamethasone 6 mg/kg 24 hours and dexamethasone 2 mg/kg 1 hour prior to first two glofitamab doses.
   FIG. 6 illustrates the in vivo study design to evaluate the effect of dexamethasone scheduling on CRS and efficacy of glofitamab.

### Sample Collection

Blood samples (20-50 μL/mouse) were collected at the following time points: 4 and 28 hours after the first and second glofitamab injection (days 14, 15, 21, and 22; 5 hr upon last dexamethasone injection). Serum was prepared by centrifugation.

### Test Item Specifications

Glofitamab (Roche):
Stock concentration: 19.7 mg/mL; buffer: 20 mM Histidine, 140 mM NaCl, 0.01% Tween 20, pH 6.0
Dexamethasone (Dexamethasone sodium phosphate):
Batch: ID279, PHR1768 provided by Sigma-Aldrich. Stock concentration 0.6 mg/ml (salt correction factor: 1.316); buffer: Saline
Gemcitabine
Batch: ID284, provided by Sandoz Sol Inf; Lot MR2763; Stock concentration: 40 mg/ml; Buffer: 20 mM Histidine, 140 mM NaCl, 0.01% Tween 20, pH 6.0
Oxaliplatin
BatchID283, provided by MedChem Express HY-17371; Lot 240620; stock concentration; buffer: 1 mg/mL 20 mM Histidine, 140 mM NaCl, 0.01% Tween 20, pH 6.0

### Tumor Growth Measurement

Tumor width and length were measured using calipers every second day; the corresponding tumor volume (TV) was calculated using the following formula: TV=(W²/2)×L, where W is width and L is length.

### Measurement of Cytokines by Multiplex Assay

Serum was collected and stored at −20° C. Serum samples were diluted 1:10 and used for the analysis of different cytokines/chemokines using the Bio-Plex Pro™ Human Chemokine Panel, 40-Plex system (Catalog No. 171AK99MR2; Bio-Rad; Hercules, CA), following the manufacturer's instructions.

### Results

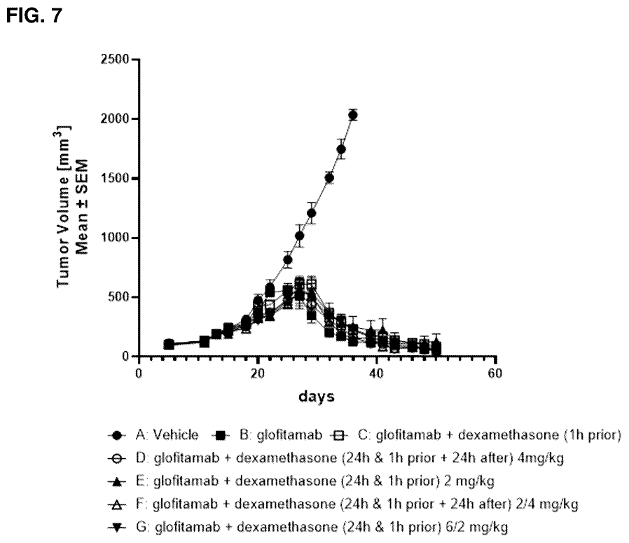

There was no difference in the inhibition of tumor growth between glofitamab monotherapy and combination therapy with dexamethasone, irrespective of the dose and scheduling in WSU-DLCL2 tumor-bearing humanized BRGS-CD47 mice (FIG. 7).

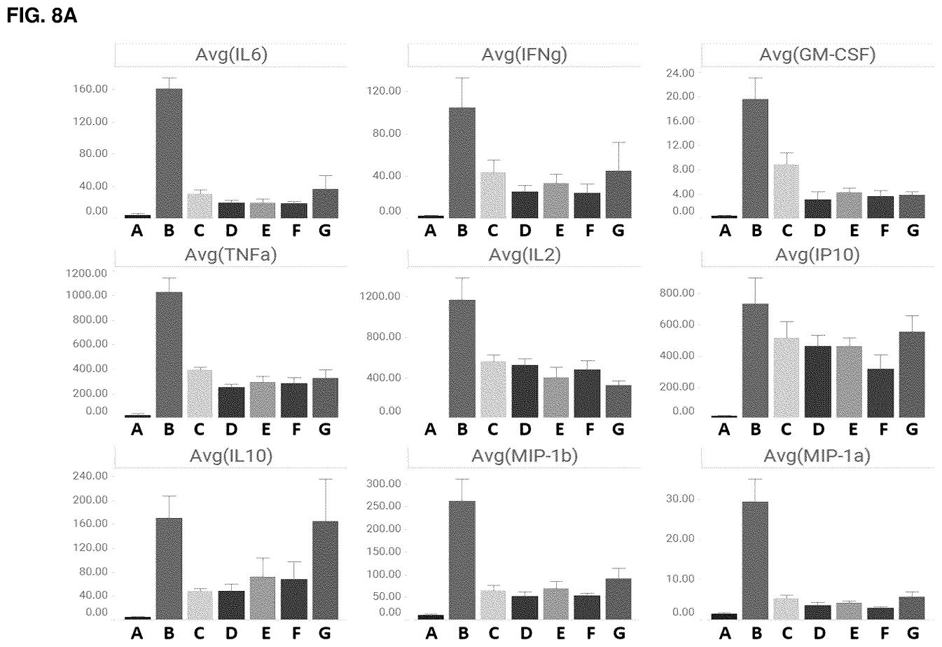

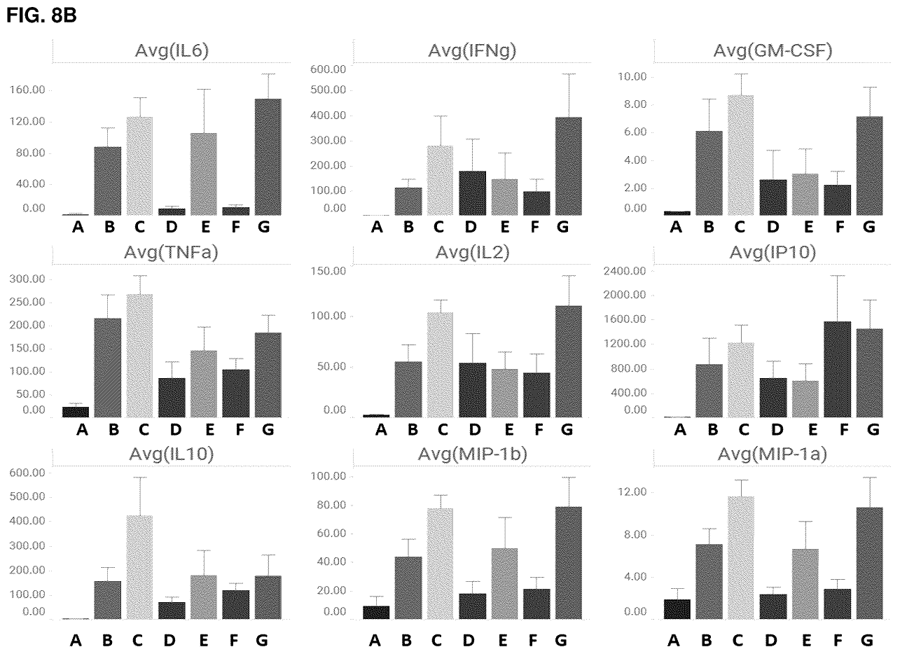

Serum cytokine measurements were conducted after the first and second glofitamab injection, at 4 and 28 hours after treatment (FIG. 8). Glofitamab monotherapy induced strong cytokine release at 4 hours after the first injection. Co-administration of dexamethasone in Groups C-G reduced cytokine levels at this early timepoint, with no major differences among groups receiving different doses and schedules of dexamethasone. The second dexamethasone dose prior to glofitamab in Groups D-G further reduced the levels of some cytokines (e.g., granulocyte-macrophage colony-stimulating factor), as compared with those after only 1 dose of dexamethasone. At 28 hours after the first glofitamab injection, glofitamab monotherapy led to higher cytokine levels in the serum. No difference in cytokine levels was observed in mice of Groups C and E that received 1 or 2 dexamethasone doses prior to glofitamab, as compared with those that only received glofitamab (Group B). This indicates the transient effect of dexamethasone in reducing cytokine levels, restricted to early timepoints (4 hours) after glofitamab infusion. The treatment groups that received dexamethasone prior to and after glofitamab injection (Groups D and F) showed very low cytokine levels also at 28 hours, reflecting the efficient dexamethasone-mediated mitigation of cytokine release for longer periods of time. The cytokines that were mainly reduced at later timepoints (in Groups D and F) were interleukin 6 (IL-6), macrophage inflammatory protein 1 (MIP1) β, MIP1α, and tumor necrosis factor alpha (TNFα), which are mostly secreted by myeloid cells (monocytes and neutrophils), reflective of the effect of dexamethasone in reducing the “amplification wave” of cytokine release by myeloid cells. Cytokine measurements after the second glofitamab injection did not show an increase in cytokine levels, which were much lower than those after the first cycle of treatment.

### CONCLUSIONS

The presented preclinical studies investigated the effects of dexamethasone premedication on CRS induced by glofitamab administered in monotherapy. Previous studies have shown that dexamethasone premedication before TCB infusion mitigates CRS without compromising the antitumor efficacy of the TCB. The current studies aimed to optimize the dosing and scheduling of dexamethasone to further reduce CRS caused by glofitamab. The administration of a standard dose of dexamethasone (20 mg) 1 hour before glofitamab infusion significantly reduced cytokine levels at 4 hours post-infusion. An additional dose of dexamethasone given 24 hours before glofitamab was similarly effective at 4 hours post-infusion and provided additional reduction in cytokine levels at 28 hours post-infusion. Posttreatment doses of dexamethasone (given 24 hours after the glofitamab infusion) were also effective in reducing cytokine levels at later timepoints. Importantly, none of the dexamethasone regimens affected the antitumor activity of glofitamab. In the second study (Example 6), the combination of glofitamab with the chemotherapy agents gemcitabine and oxaliplatin was evaluated. These results are described below.

### Example 6. Preclinical Study to Evaluate Effect of Dexamethasone Scheduling on CRS and Efficacy of Glofitamab in Combination with Gemcitabine and Oxaliplatin

The efficacy study described herein aimed to assess the impact of dexamethasone on Glofitamab induced cytokine release syndrome (CRS) and long-term efficacy in combination with gemcitabine and oxaliplatin in a CD20-positive human lymphoma model using fully humanized BRGS-CD47 mice.

Human OCI-Ly18 (Diffuse Large B-Cell Lymphoma; DLBCL) cells were originally obtained from ATCC and subsequently expanded and deposited in the Roche Glycart internal cell bank. Cells were cultured in RPMI medium containing 10% fetal calf serum (FCS) and 1× GLUTAMAX®. The cells were maintained at 37° C. in a water-saturated atmosphere with 5% CO2. A 50 μL cell suspension (5×10\hat{ }6 OCI-Ly18 cells) mixed with 50 μL Matrigel was injected subcutaneously into the flank of anesthetized mice using a 22G to 30G needle.

BRGS-CD47 female mice (immunodeficient mice expressing human SIRPA and CD47, developed by Roche and bred by Jackson Laboratories, Sacramento, CA) were humanized at 3-4 weeks of age by total body irradiation with 200 cGy, followed by intravenous injection of 10\hat{ }5 human hematopoietic stem cells. Twelve to fifteen weeks after stem cell injection, humanized BRGS-CD47 mice were screened for human cell engraftment in the blood by flow cytometry. The humanization and screening process was conducted at Jackson Laboratory, and only mice with a humanization rate greater than 20% (i.e., more than 20% of circulating human immune cells within all leukocytes) were shipped to Roche Innovation Center Zurich for subsequent experiments. Upon arrival, mice were maintained under specific-pathogen-free conditions and a 12-hour light/dark cycle according to committed guidelines (GV-Solas, Felasa, TierschG). The experimental study protocol was reviewed and approved by the local government (License ZH180-20). Health monitoring was performed regularly.

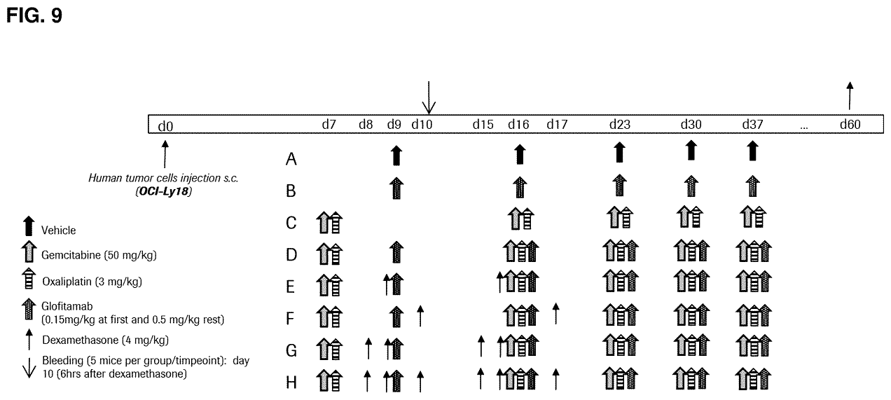

At the appropriate time, mice were injected subcutaneously with tumor cells as described and treated with the compounds or histidine buffer (Vehicle; Group A) when tumor size reached approximately 200 mm³ (Day 7). FIG. 9 and Table 19 describe the details of the administration schedules and routes of administration for glofitamab, gemcitabine, oxaliplatin, and dexamethasone. Gemcitabine and oxaliplatin were injected intraperitoneally (i.p.), glofitamab intravenously (i.v.), and dexamethasone was administered via oral gavage.

Groups A-D received no dexamethasone:
Group A: vehicle (control group).
Group B: glofitamab monotherapy
Group C: gemcitabine and oxaliplatin.
Group D: glofitamab, gemcitabine and oxaliplatin.
   Groups E-H received the combination treatment along with dexamethasone at different schedules:
Group E: 1 hour prior to first two doses of glofitamab
Group F: 24 hours post first two doses of glofitamab.
Group G: 24 hours and 1 hour prior to first two doses of glofitamab.
Group H: 24 hours and 1 hour prior, as well as 24 hours post first two doses of glofitamab treatment

TABLE 19
Administration schedules and routes of administration for
glofitamab, gemcitabine, oxaliplatin, and dexamethasone

| | No. of | | | Injection | |
|---|---|---|---|---|---|
| Group | animals | Compound | Dose (mg/kg) | route | No. of treatments |
| A | 12 | Vehicle | — | i.v. | 7 (once weekly) |
| B | 12 | Glofitamab | 0.15 (first)/0.5 (rest) | i.v. | 1 (0.15 mg/kg once) + 6 |
| | | | | | (0.5 mg/kg once weekly) |
| C | 12 | Gemcitabine + | 50/3 | i.p. | 7 (once weekly) |
| | | Oxaliplatin | | | |
| D | 12 | Glofitamab + | 0.15 (first)/0.5 (rest) | i.v. | 1 (0.15 mg/kg once) + |
| | | Gemcitabine + | 50/3 | i.p. | 6 (0.5 mg/kg once weekly) |
| | | Oxaliplatin | | | 7 (once weekly) |
| E | 12 | Glofitamab + | 0.15 (first)/0.5 (rest) | i.v. | 1 (0.15 mg/kg once) + |
| | | Gemcitabine + | 50/3 | i.p. | 6 (0.5 mg/kg once weekly) |
| | | Oxaliplatin + | Dexamethasone: 4 | p.o. | 7 (once weekly) |
| | | Dexamethasone | | | 2 (once at first and |
| | | (1 hr prior | | | second cycle) |
| | | Glofitamab) | | | |
| F | 12 | Glofitamab + | 0.15 (first)/0.5 (rest) | i.v. | 1 (0.15 mg/kg once) + |
| | | Gemcitabine + | 50/3 | i.p. | 6 (0.5 mg/kg once weekly) |
| | | Oxaliplatin + | Dexamethasone: 4 | p.o. | 7 (once weekly) |
| | | Dexamethasone | | | 2 (once at first and |
| | | (24 hr post | | | second cycle) |
| | | CD20-TCB) | | | |
| G | 12 | CD20-TCB + | 0.15 (first)/0.5 (rest) | i.v. | 1 (0.15 mg/kg once) + |
| | | Gemcitabine + | 50/3 | i.p. | 6 (0.5 mg/kg once weekly) |
| | | Oxaliplatin + | Dexamethasone: 4 | p.o. | 7 (once weekly) |
| | | Dexamethasone | | | 4 (twice at first and |
| | | (24 hr and 1 hr | | | second cycle) |
| | | prior CD20-TCB) | | | |
| H | 12 | CD20-TCB + | 0.15 (first)/0.5 (rest) | i.v. | 1 (0.15 mg/kg once) + |
| | | Gemcitabine + | 50/3 | i.p. | 6 (0.5 mg/kg once weekly) |
| | | Oxaliplatin + | Dexamethasone: 4 | p.o. | 7 (once weekly) |
| | | Dexamethasone | | | 6 (thrice at first and |
| | | (24 hr and 1 hr | | | second cycle) |
| | | prior and 24 hr | | | |
| | | post CD20-TCB) | | | |

All mice were injected with the appropriate solutions. For all combination groups, chemotherapy was administered first on Day 7, followed by glofitamab on Day 9. Subsequent combination treatments were carried out concomitantly. To obtain the proper amount of compounds per injection volume, the stock solutions (Table 20) were diluted with histidine buffer when necessary.

Mice were bled and serum prepared for cytokines analysis at day 10 (6 hrs after last dexamethasone injection in group F and H). Cytokine measurement was carried out with the Luminex™ FLEXMAP 3D™ system using Bio-Plex Pro Human Cytokine Assay according to the manufacturer's instruction.

Tumor growth was measured three times weekly using a caliper and tumor volume was calculated as followed:

$${T}_{v} : ( {W}^{2} / 2 ) \times L \qquad ( W : Width , L : Length )$$

The study was terminated at day 60 after a total of 7 cycles of treatment.

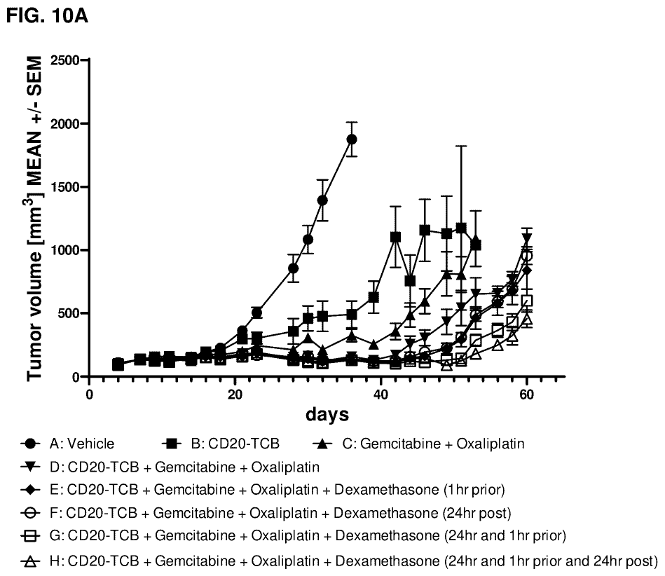

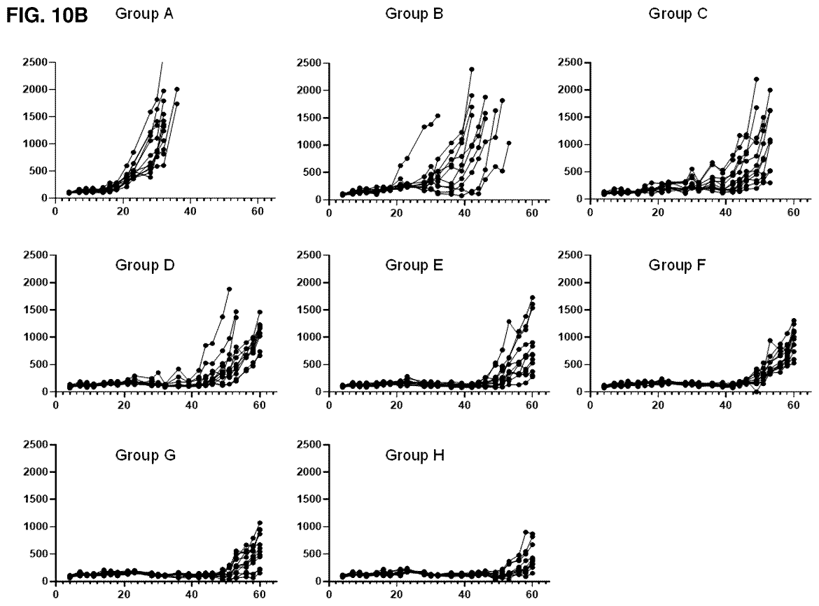

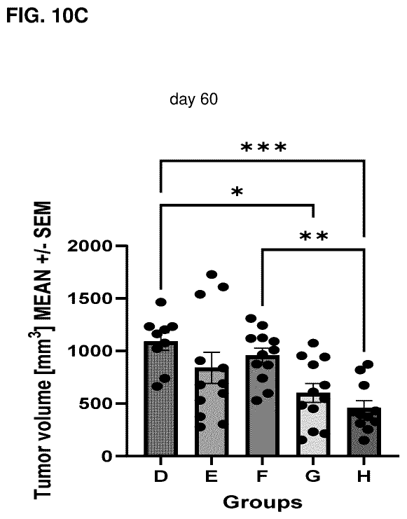

FIG. 10 illustrates the tumor growth kinetics as the mean tumor volume (FIG. 10A), individual tumor growth trajectories for each mouse within the various treatment groups (FIG. 10B), and the tumor volumes from Groups D-H at study termination for statistical analysis (FIG. 10C). The data indicate that monotherapy with glofitamab (Group B) initially elicited a robust inhibition of tumor growth, which was subsequently followed by tumor recurrence in all treated mice. Monotherapy with the chemotherapy treatment also resulted in robust anti-tumor effects, but the majority of mice experienced tumor regrowth by Day 60.

The combination of chemotherapy and glofitamab (Group D) showed some prolongation of the anti-tumor effect, but tumor regrowth was still observed by study termination. Interestingly, the groups treated with dexamethasone (Groups E-H) demonstrated an improvement in anti-tumor effects, which correlated with the frequency of dexamethasone administration. Groups G and H, which received the most frequent dexamethasone injections, exhibited statistically significant better anti-tumor effects compared to the combination treatment of CD20-TCB and chemotherapy without dexamethasone.

TABLE 20
Stock solutions used in preclinical studies

| | Formulation | Concentration |
|---|---|---|
| Compound | buffer | (mg/mL) |
| glofitamab | 20 mM Histidine, | 19.7 |
| (ID: P1AA4006-37290) | 140 mM NaCl, | (=stock solution) |
| | pH 6.0 | |
| Dexamethasone | Saline | 0.6 mg/mL |
| Sodium phosphate (ID: | | (salt correction |
| 279, PHR1768 | | factor: 1.316) |
| Sigma-Aldrich) | | (=stock solution) |
| Gemcitabine (ID284, | 20 mM Histidine, | 40 |
| Sandoz Sol Inf; | 140 mM NaCl, | (=stock solution) |
| Lot MR2763) | pH 6.0 | |
| Oxaliplatin (ID283, | 20 mM Histidine, | 1 |
| MedChem Express | 140 mM NaCl, | (=stock solution) |
| HY-17371; Lot 240620) | pH 6.0 | |

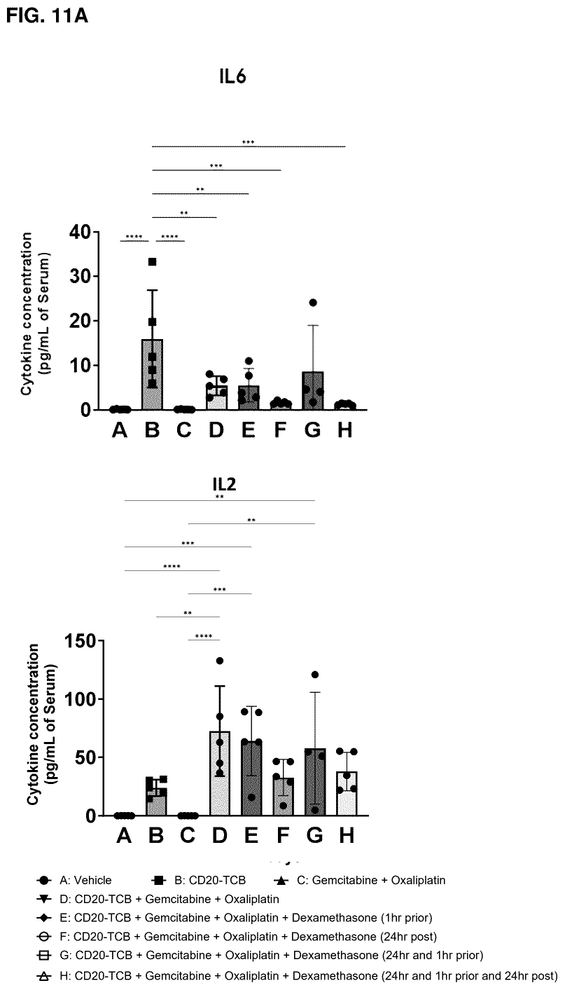

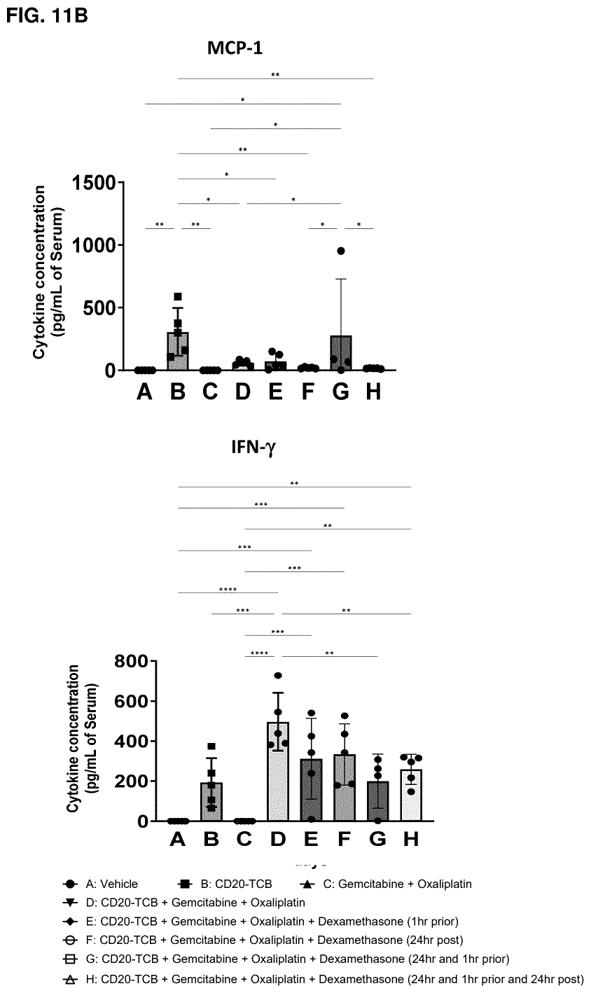

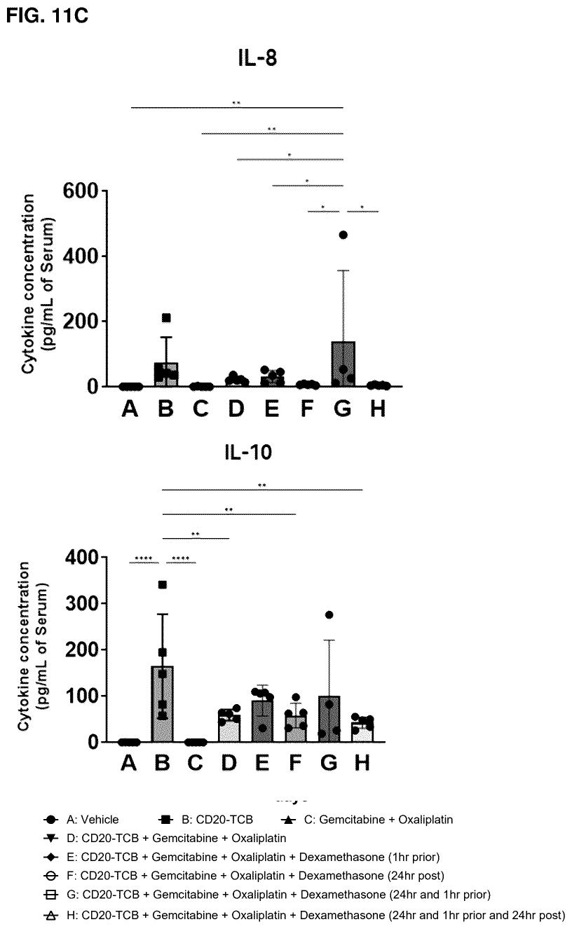

Furthermore, as displayed in FIG. 11, the analysis of serum cytokines on day 10 (6 hours after the last dexamethasone injection in Groups F and H) revealed a significant reduction in CRS-related cytokines, such as IL-6, in the groups receiving an additional dexamethasone injection 24 hours post glofitamab injection (Groups F and H) compared to the glofitamab monotherapy group. Chemotherapy treatment did not trigger any measurable cytokine levels in the serum of mice. Other statistically reduced cytokines included MCP-1 and IL-8, whereas IFN-γ and IL-2 did not show as strong a reduction in those groups.

These preclinical studies demonstrate that dexamethasone pre-medication reduces glofitamab induced cytokine release and has no negative impact on the glofitamab mediated antitumor efficacy, supporting its clinical use to mitigate CRS.

The additional dexamethasone premedication dose (24-hour pre-glofitamab administration) reduced IL-6, and to some extent IL-10 levels, at the early time point, with no mitigation capacity at the later time point. The addition of dexamethasone post treatment dose (e.g., 24 hours post-glofitamab administration) mitigated IL-6 at the later time point (28 hours post-glofitamab administration).

Importantly, the anti-tumor efficacy of glofitamab monotherapy and of glofitamab in combination with gemcitabine and oxaliplatin was not affected.

Taken together, the findings of the current preclinical studies point to the relevance of dexamethasone pre- and post-treatment doses to further optimize glofitamab-mediated cytokine release, without negatively affecting the anti-tumor efficacy of the Glofit-GemOx regimen.

### Example 7 A Phase II, Open-Label, Multicenter Study to Evaluate the Optimization of the Cytokine Release Syndrome Profile for Glofitamab in Combination with Gemcitabine Plus Oxaliplatin in Patients with Relapsed/Refractory Diffuse Large B-Cell Lymphoma (GO45434)

### Study Rationale

The purpose of this study is to evaluate the optimization of the cytokine release syndrome (CRS) profile for the glofitamab plus gemcitabine-oxaliplatin (Glofit-GemOx) treatment regimen in patients with relapsed or refractory (R/R) diffuse large B cell lymphoma (DLBCL). An optimized administration dose of steroids, and a revised infusion and monitoring regimen to optimize the CRS profile of this treatment combination, are utilized in this study. Prior studies have evaluated glofitamab in R/R DLBCL, both as monotherapy and various combinations, including with gemcitabine-oxaliplatin in the STARGLO/GO41944 randomized Phase III study (Examples 1-4). In STARGLO/GO41944, the most recent CRS data is as follows: Any Grade: 44.2%, Grade 1:31.4%, Grade 2:10.5%, Grade 3:2.3%, the CRS occurred primarily in Cycle 1. Preclinical data supports the clinical use of dexamethasone as a premedication strategy to reduce the side effects of glofitamab therapy while maintaining its therapeutic benefits (Examples 5 and 6).

TABLE 21
Objectives and endpoints

| Objectives | Endpoints |
|---|---|
| Primary Objective | Corresponding Endpoint |
| To evaluate the frequency of CRS | Incidence of CRS, defined as the number of participants with |
| | the following: |
| | Grade 2 or higher CRS at any time during the study |
| | Any Grade CRS, at any time during the study |
| Secondary Objectives | Corresponding Endpoint |
| To evaluate the frequency and severity of | Incidence of serious CRS events, with severity determined |
| CRS | according to the ASTCT CRS grading criteria |
| | CRS frequency relative to the start of the infusion of each |
| | of the step-up doses of glofitamab |
| | CRS management, including outcome of patients who |
| | experienced a CRS event |
| To evaluate the safety of Glofit-GemOx | Incidence and severity of adverse events, with severity |
| | determined according to the NCI CTCAE v5.0 |
| | Tolerability, as assessed by dose interruptions, dose |
| | reductions, and dose intensity |
| | Study treatment discontinuation because of adverse |
| | events |
| To evaluate the efficacy of Glofit-GemOx | CR rate, defined as the proportion of patients whose best |
| | overall response is a CR on PET/CT during the study, as |
| | determined by the investigator |
| | ORR, defined as the proportion of patients whose best |
| | overall response is a PR or a CR during the study, as |
| | determined by the investigator |
| | DOR, defined as the time from the first occurrence of a |
| | documented objective response (CR or PR) to disease |
| | progression, or death from any cause, whichever occurs |
| | first |
| | DOCR, defined as the time from the first occurrence of a |
| | documented CR to disease progression, or death from any |
| | cause, whichever occurs first |
| | PFS, defined as the time from enrollment to the first |
| | occurrence of disease progression or death from any |
| | cause, whichever occurs first, as determined by the |
| | investigator |
| | OS, defined as the time from enrollment to date of death |
| | from any cause |
| | Note: All response assessments are based on the 2014 |
| | Lugano Response Criteria |
| Exploratory Objectives | Corresponding Endpoint |
| To evaluate the efficacy of Glofit-GemOx | Descriptive summary statistics of PROs and the change |
| | from baseline at each assessment for the following: |
| | All scales and items of the EORTC QLQ-C30 |
| | FACT-Lym LymS |
| To characterize the pharmacokinetics of | Serum concentration of glofitamab at specified time points |
| glofitamab when administered as a | Obtain PK and exposure parameters by including data in |
| component of Glofit-GemOx | existing population-PK model |
| To evaluate the immune response to | Prevalence of ADAs against glofitamab at baseline and |
| Glofit-GemOx | incidence of ADAs during the study |
| To evaluate the tolerability of | Presence, frequency of occurrence, severity, and/or |
| Glofit-GemOx from the participant's | degree of interference with daily function of symptomatic |
| perspective | treatment toxicities (specify as relevant for treatments |
| | included in study, i.e., Glofitamab, gemcitabine, |
| | oxaliplatin), as assessed through use of the NCI |
| | PRO-CTCAE |
| | Frequency of participants' response of the degree to which |
| | they are bothered by treatment symptoms, as assessed |
| | through use of the FACT-G GP5 |

ADA = anti-drug antibodies; ASTCT = American Society for Transplantation and Cellular Therapy; CR = complete response; CRS = cytokine release syndrome; CT = computed tomography; DOCR = duration of complete response; DOR = duration of response; EORTC QLQ-C30 = European Organisation for Research and Treatment of Cancer Quality of Life-Core 30 Questionnaire; FACT G GP5 = Functional Assessment of Cancer Therapy-General; FACT-Lym LymS = Functional Assessment of Cancer Therapy-Lymphoma Lymphoma subscale; GemOx = gemcitabine and oxaliplatin; Glofit = glofitamab; NCI CTCAE = National Cancer Institute Common Toxicity Criteria for Adverse Events; ORR = overall response rate; OS = overall survival; PET = positron emission tomography; PFS = progression-free survival; PK = pharmacokinetic; PR = partial response; PRO = patient-reported outcome.

### Overall Design and Study Population

This is a Phase II, open-label, multicenter study to evaluate the optimization of the CRS profile for the glofitamab plus Glofit-GemOx treatment regimen in patients with R/R DLBCL, using an optimized regimen of steroids and a revised infusion and monitoring regimen to optimize the CRS profile of this treatment combination.

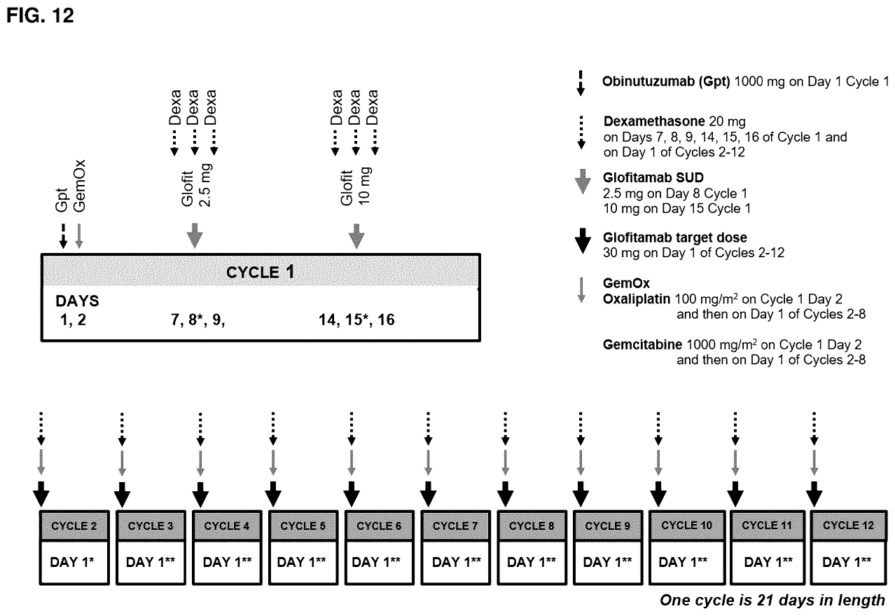

Patients receive a single IV dose of obinutuzumab pretreatment (Gpt) 7 days before the first dose of glofitamab, then up to 8 cycles of glofitamab in combination with gemcitabine plus oxaliplatin, followed by up to 4 cycles of glofitamab monotherapy, to complete up to a total of 12 cycles of glofitamab, with each cycle being 21 days (3 weeks). FIG. 12 illustrates the dosing regimen for Glofit-GemOx.

An optimized steroid regimen is administered for the glofitamab step doses of 2.5 mg and 10 mg, which includes 20 mg of dexamethasone orally one day prior to the glofitamab administration, 20 mg of dexamethasone IV 60 minutes prior to the glofitamab administration and 20 mg of dexamethasone orally one day after the glofitamab administration.

Several key aspects of the study design and study population are summarized below. FIG. 12 shows the study schema.

TABLE 22
Study design and study population

| | | | |
|---|---|---|---|
| Phase: | Phase II | Population Type: | Adult patients |
| Control Method: | None | Population Diagnosis or | Relapsed or refractory |
| | | Condition: | diffuse large B cell |
| | | | lymphoma |
| Interventional Model: | Single group | Population Age: | ≥18 years |
| Test Products: | Glofitamab | Site Distribution: | Multi-site |
| | Obinutuzumab | | |
| | Tocilizumab | | |
| Active Comparator: | Not Applicable | Study Treatment | Non-randomized, |
| | | Assignment Method: | open-label |
| Number of Arms: | One | Number of Participants to | Approximately 50 |
| | | Be Enrolled: | participants |

### Study Treatment

### Glofitamab

Glofitamab is administered intravenously on a step-up dosing schedule, starting with 2.5 mg on Day 8 of Cycle 1, 10 mg on Day 15 of Cycle 1, and then 30 mg on Day 1 of Cycles 2-12. Glofitamab is administered to well-hydrated participants. The infusion of glofitamab is completed in observational units, infusion centers, or equivalent outpatient settings with immediate access to trained critical care personnel and facilities equipped to respond to and manage medical emergencies. For the glofitamab step doses of 2.5 mg and 10 mg, participants receive 20 mg of dexamethasone orally one day prior to the glofitamab administration, 20 mg of dexamethasone IV 60 minutes prior to the glofitamab administration, and 20 mg of dexamethasone orally one day after the glofitamab administration. For the target dose of 30 mg glofitamab, 20 mg dexamethasone is administered IV 60 minutes prior to glofitamab administration. Premedication with acetaminophen or paracetamol (500-1000 mg) and an antihistamine, such as diphenhydramine (50 mg), is administered approximately 30 minutes prior to the start of the infusion. Dexamethasone premedication is optional at later cycles based on investigator's assessment for participants who have tolerated at least one 30 mg dose of glofitamab without experiencing CRS. However, if the participant experiences CRS, premedication with dexamethasone is required for subsequent glofitamab doses until no additional CRS events are observed.

### Obinutuzumab

Obinutuzumab pretreatment is administered by IV infusion as an absolute (flat) dose of 1000 mg on Day 1 of Cycle 1 (7 days prior to the first administration of glofitamab) for all participants. Obinutuzumab should be administered to well hydrated participants. Premedication with dexamethasone 20 mg IV or methylprednisolone 80 mg IV is required and is administered 1 hour prior to the administration of obinutuzumab; premedication with oral acetaminophen or paracetamol (500 or 1000 mg) and an antihistamine, such as diphenhydramine (50 mg), is administered approximately 30 minutes prior to the start of the infusion.

### Tocilizumab

Tocilizumab is administered as a rescue IMP when necessary to participants who experience a CRS event. Tocilizumab is administered at dose of 8 mg/kg IV (not exceeding 800 mg per infusion). If no clinical improvement in the signs and symptoms of CRS occurs after the first dose, a second dose may be administered at least 8 hours apart (maximum 2 doses per CRS event). Within each time period of 6 weeks of glofitamab treatment, the total number of tocilizumab doses does not exceed 3 doses.

### Gemcitabine

Gemcitabine is administered intravenously to participants at 1000 mg/m² on Cycle 1 Day 2. Starting at Cycle 2, gemcitabine may be given on Day 1 or 2 (per local practice) of each 21-day cycle (Cycles 2-8). Gemcitabine is administered before oxaliplatin on the same day.

### Oxaliplatin

Oxaliplatin is administered intravenously to participants at 100 mg/m² on Cycle 1 Day 2. Starting at Cycle 2, gemcitabine may be given on Day 1 or 2 (per local practice) of each 21-day cycle (Cycles 2-8). Oxaliplatin should be administered after gemcitabine on the same day.

Modification of the glofitamab, obinutuzumab, or tocilizumab is not permitted; gemcitabine dosing should not be modified unless recommended by local prescribing information. The dose of oxaliplatin can be reduced for management of drug-related toxicities.

### Duration of Participation

The total duration of study participation for each individual is expected to be up to 5 years.

### Committees

An Internal Monitoring Committee (IMC) is used during the study to make recommendations on study conduct based on trial safety data, to ensure enhanced participant safety during study treatment.

FIG. 12 shows the study schema.

Study GO41944 (Examples 1 to 4) required pretreatment with a single dose of dexamethasone 20 mg IV at least 60 minutes prior to glofitamab doses until patients tolerated two 30 mg doses of glofitamab without experiencing CRS, at which point dexamethasone became optional. For the optimized treatment regimen, for the glofitamab step-up doses of 2.5 mg and 10 mg, participants receive an increased dexamethasone dosing regimen:
20 mg of dexamethasone orally on the day prior to the glofitamab administration,
20 mg of dexamethasone IV at least 60 minutes prior to the glofitamab administration,
20 mg of dexamethasone orally on the day after the glofitamab administration.

### Inclusion Criteria

Potential participants are eligible to be included in the study only if all of the following criteria apply:
Signed Informed Consent Form.
Age ≥18 years at the time of signing Informed Consent Form
Ability and willingness to comply with the study protocol

$$Life expectancy \geq 12 weeks$$

Histologically confirmed DLBCL, not otherwise specified (NOS)
R/R disease, defined as follows:
   Relapsed: disease that has recurred following a response that lasted ≥6 months after completion of the last line of therapy<
   Refractory: disease that did not respond to or that progressed <6 months after completion of the last line of therapy

Participants who discontinued the last line of therapy before sufficient time for response assessment (e.g., due to toxicity) are assessed for refractoriness based on the previous line of therapy
At least one prior line of systemic therapy
   Participants may have undergone ASCT prior to recruitment
   Local therapies (e.g., radiotherapy) are not considered as lines of therapy.
Participants who have failed only one prior line of therapy must not be a candidate for high-dose chemotherapy followed by ASCT, by meeting at least one of the following criteria:

$$\begin{matrix}Left ventricular ejection fraction \leq 40 \% \\ CrCl or glomerular filtration rate \leq 45 mL / min\end{matrix}$$

ECOG Performance Status of 2

$$Age \geq 70 years$$

Participant refused high-dose chemotherapy and/or ASCT
Participant had insufficient response to pre-transplant chemotherapy to be able to proceed to ASCT
Other comorbidities or criteria that preclude the use of transplant based on local practice standards or in the investigator's opinion. The rationale for transplant ineligibility must be recorded in the electronic Case Report Form (eCRF).
At least one bi-dimensionally measurable (>1.5 cm) nodal lesion, or one bi dimensionally measurable (>1 cm) extranodal lesion, as measured on CT scan
ECOG Performance Status of 0, 1, or 2
Adequate hematologic function (unless attributable to the underlying disease, as established by extensive bone marrow involvement or associated with hypersplenism secondary to the involvement of the spleen by DLBCL per the investigator), defined as follows:

$$ANC \geq 0.75 \times {10}^{9} / L ( 750 / μL )$$

Platelet count ≥50×10⁹/L (50,000/μL) without a transfusion in the week prior to starting study treatment
Adequate renal function, defined as CrCl ≥30 mL/min.
Negative SARS-COV-2 antigen or PCR test within 7 days prior to enrollment.
Negative HIV test at screening, with the following exception: individuals with a positive HIV test at screening are eligible provided they are stable on anti-retroviral therapy, have a CD4 count ≥200/μL, have an undetectable viral load, and have not had a history of opportunistic infection attributable to AIDS within the last 12 months.
For female participants of childbearing potential: agreement to remain abstinent (refrain from heterosexual intercourse) or use contraception, and agree to refrain from donating eggs, as defined below:
   Female participants must remain abstinent or use contraceptive methods with a failure rate of <1% per year during the treatment period and for at least 18 months after the final dose of obinutuzumab, 9 months after the final dose of oxaliplatin, 6 months after the final dose of gemcitabine, 2 months after the final dose of tocilizumab (if applicable), and 1 month after the final dose of glofitamab. Participants must refrain from donating eggs during this same period.
   A female participant is considered to be of childbearing potential if she is postmenarchal, has not reached a postmenopausal state (≥12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). Per this definition, a female participant with tubal ligation is considered to be of childbearing potential. The definition of childbearing potential may be adapted for alignment with local guidelines or regulations.
   Examples of contraceptive methods with a failure rate of <1% per year include bilateral tubal ligation, male sterilization, hormonal contraceptives that inhibit ovulation, hormone-releasing intrauterine devices, and copper intrauterine devices.
   The reliability of sexual abstinence should be evaluated in relation to the duration of the clinical trial and the preferred and usual lifestyle of the individual. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence is described in the local Informed Consent Form.
For male participants: agreement to remain abstinent (refrain from heterosexual intercourse) or use a condom, and agree to refrain from donating sperm, as defined below:
   With a female partner of childbearing potential or pregnant female partner, men must remain abstinent or use a condom during the treatment period and for at least 6 months after the final dose of oxaliplatin or gemcitabine, 2 months after the final dose of tocilizumab (if applicable), and 1 month after the final dose of glofitamab to avoid exposing the embryo. Male participants must refrain from donating sperm during this same period.
   The reliability of sexual abstinence should be evaluated in relation to the duration of the clinical trial and the preferred and usual lifestyle of the individual. Periodic abstinence (e.g., calendar, ovulation, symptothermal, or postovulation methods) and withdrawal are not adequate methods of preventing drug exposure. If required per local guidelines or regulations, information about the reliability of abstinence is described in the local Informed Consent Form.

### Exclusion Criteria

Potential participants are excluded from the study if any of the following criteria apply:
Prior enrollment in Study GO41943 (NCT04313608), GO41944 (STARGLO; NCT04408638) or Study GO44900.
Participant has failed only one prior line of therapy and is a candidate for stem cell transplantation.
History of transformation of indolent disease to DLBCL.
High-grade B-cell lymphoma with MYC and BCL2 and/or BCL6 rearrangements, and high-grade B-cell lymphoma NOS, as defined by 2016 WHO guidelines (Swerdlow et al. 2016)
   Patients classified as “double-hit” lymphoma with known MYC and BCL2 and/or BCL6 rearrangements are excluded.
   Patients with DLBCL characterized as “double expressors” without the above rearrangements are not excluded.
Primary mediastinal B-cell lymphoma.
History of severe allergic or anaphylactic reactions to humanized or murine monoclonal antibodies (or recombinant antibody-related fusion proteins) or known sensitivity or allergy to murine products.
Contraindication to obinutuzumab, gemcitabine or oxaliplatin, or tocilizumab.
Prior treatment with glofitamab or other bispecific antibodies targeting both CD20 and CD3.
Prior treatment with gemcitabine or oxaliplatin.
Peripheral neuropathy or paresthesia assessed to be Grade ≥2 according to NCI CTCAE v5.0 at enrollment.
Treatment with radiotherapy, chemotherapy, immunotherapy, immunosuppressive therapy, or any investigational agent for the purposes of treating cancer within 2 weeks prior to first study treatment.
Treatment with monoclonal antibodies for the purposes of treating cancer within 4 weeks prior to first study treatment.
Primary or secondary CNS lymphoma at the time of recruitment or history of CNS lymphoma.
Prior CNS involvement that has been definitively treated and confirmed via magnetic resonance imaging (MRI) or cerebrospinal fluid analysis to be in complete remission is permissible.
Current or history of CNS disease, such as stroke, epilepsy, CNS vasculitis, or neurodegenerative disease.
   Participants with a history of stroke who have not experienced a stroke or transient ischemic attack within the past 2 years and have no residual neurologic deficits, as judged by the investigator, are allowed.
Any of the following abnormal laboratory values, unless abnormal laboratory values are associated with the underlying lymphoma per the investigator:

$$\begin{matrix}AST or ALT > 2.5 \times upper limit of normal ( ULN ) \\ Total bilirubin \leq 1.5 \times ULN\end{matrix}$$

Participants with documented Gilbert disease may be enrolled if the total bilirubin is ≥3×ULN

$$INR or PT > 1.5 \times ULN , in the absence of therapeutic anticoagulation$$

$$PTT or aPTT > 1.5 \times ULN in the absence of therapeutic anticoagulation or a lupus anticoagulant$$

History of other primary malignancy, with the following exceptions:
   Participants with a history of curatively treated basal or squamous cell carcinoma of the skin or in situ carcinoma of the cervix at any time prior to the study are eligible.
   Participants with low grade, early-stage prostate cancer (Gleason score 6 or below, Stage 1 or 2) with no requirement for therapy at any time prior to study are eligible.
   Participants with any other malignancy appropriately treated with curative intent and the malignancy has been in remission without treatment for >2 years prior to enrollment are eligible.
   Participants receiving adjuvant endocrine therapy for non-metastatic, hormone receptor-positive breast cancer for ≥2 years prior to enrollment are eligible.
Significant or extensive cardiovascular disease such as New York Heart Association Class III or IV cardiac disease or Objective Assessment Class C or D, myocardial infarction within the last 3 months, unstable arrhythmias, or unstable angina
Significant pulmonary disease (including moderate or severe obstructive pulmonary disease)
Known active bacterial, viral, fungal, mycobacterial, parasitic, or other infection (excluding fungal infections of nail beds) at study enrollment or any major episode of infection (as evaluated by the investigator) within 4 weeks prior to the first study treatment
Documented severe acute respiratory syndrome coronavirus 2 (SARS-COV-2) infection within 6 months of first study treatment
   Participants may be eligible if they have no persistent respiratory symptoms, no evidence of lung infiltrates on chest CT, and have a negative PCR during the 7 days prior to first study treatment.
Positive SARS-COV-2 test within 7 days prior to enrollment. Rapid antigen test results are acceptable.
Suspected or latent tuberculosis (confirmed by positive IFN-γ release assay).
Positive test results for hepatitis B virus (HBV) infection (defined as positive hepatitis B surface antigen [HBsAg] serology).
   Participants with occult or prior HBV infection (defined as negative HBsAg and positive hepatitis B core antibody [HBcAb]) may be included if HBV DNA is undetectable, provided that they are willing to undergo DNA testing on Day 1 of every cycle and every three months for at least 12 months after the last cycle of study treatment and appropriate antiviral therapy.
Positive test results for hepatitis C virus (HCV) antibody

Participants who are positive for HCV antibody are eligible only if PCR is negative for HCV RNA.
Known or suspected chronic active Epstein-Barr viral infection
Known or suspected history of hemophagocytic lymphohistiocytosis (HLH)
Known history of progressive multifocal leukoencephalopathy.
Adverse events from prior anti-cancer therapy that have not resolved to Grade 1 or better (with the exception of alopecia and anorexia)
Administration of a live, attenuated vaccine within 4 weeks before first study treatment administration or anticipation that such a live, attenuated vaccine is required during the study
Prior solid organ transplantation
Prior allogeneic stem cell transplant
Active autoimmune disease requiring treatment
   Participants with a history of autoimmune-related hypothyroidism on a stable dose of thyroid-replacement hormone may be eligible.
   Participants with controlled Type 1 diabetes mellitus who are on an insulin regimen are eligible for the study.
   Participants with a history of autoimmune hepatitis, systemic lupus erythematosus, inflammatory bowel disease, vascular thrombosis associated with antiphospholipid syndrome, Wegener granulomatosis, Sjögren syndrome, multiple sclerosis, or glomerulonephritis are excluded.
   Participants with a history of immune thrombocytopenia purpura, autoimmune hemolytic anemia, Guillain-Barre syndrome, myasthenia gravis, myositis, rheumatoid arthritis, vasculitis, or other autoimmune diseases are excluded unless they have not required systemic therapy in the last 12 months.
Prior treatment with systemic immunosuppressive medications (including, but not limited to, cyclophosphamide, azathioprine, methotrexate, thalidomide, and anti-tumor necrosis factor agents), within 4 weeks prior to first dose of study treatment.
Ongoing systemic corticosteroid use which, in the opinion of the investigator, puts the participant at increased risk of steroid-related iatrogenic adrenal insufficiency.
   The use of mineralocorticoids for management of orthostatic hypotension is permitted.
   Participants with a history of pre-existing adrenal insufficiency are allowed to receive physiologic doses of corticosteroids.
   The use of inhaled corticosteroids is permitted.
Recent major surgery (within 4 weeks before the first study treatment) other than for diagnosis
Clinically significant history of cirrhotic liver disease.
Any other diseases, metabolic dysfunction, physical examination finding, or clinical laboratory finding giving reasonable suspicion of a disease or condition that contraindicates the use of an investigational drug or that may affect the interpretation of the results or renders the participant at high-risk from treatment complications.
Pregnancy or breastfeeding, or intention of becoming pregnant during the study or within 18 months after the final dose of study treatment
   Female participants of childbearing potential must have a negative serum pregnancy test result within 7 days prior to initiation of study treatment.
Lack support for close monitoring, outside the treatment setting for 24 hours following 2.5 mg and 10 mg of glofitamab dose.

### Study Treatment

The study treatment is summarized below in Table 23.

TABLE 23
Study Treatment Description

| | Glofitamab | Tocilizumab | Obinutuzumab | Gemcitabine | Oxaliplatin |
|---|---|---|---|---|---|
| Dosage level(s) | Cycle 1 Day 8: 2.5 mg | 8 mg/kg | 1000 mg | 1000 mg/m² | 100 mg · m² |
| | Cycle 1 Day 15: 10 mg | | Day −7 | | |
| | Cycles 2-12 Day 1: 30 mg | | (prior to | | |
| | | | Cycle 1) | | |
| Route of | IV | IV | IV | IV | IV |
| administration | infusion | infusion | infusion | infusion | infusion |

### Obinutuzumab

A single dose of obinutuzumab 1000 mg pretreatment is administered intravenously on Cycle 1 Day 1 (7 days prior to the first administration of glofitamab). Obinutuzumab must be administered in a clinic or hospital equipped for systemic (IV) cancer treatment. Full emergency resuscitation facilities are immediately available, and participants are under close observation by the investigator at all times. In case of infusion-associated adverse events in participants, the signs and symptoms are fully resolved before the participant is discharged. The obinutuzumab infusion may be split over 2 days if a participant is at increased risk for an infusion-related reaction (IRR) (e.g., because of high tumor burden, high peripheral lymphocyte count) or experiences an adverse event during the infusion.

Participants receive premedication prior to obinutuzumab infusion as indicated in Table 24 below. Obinutuzumab is administered to well-hydrated participants. Participants at risk of TLS receive TLS prophylaxis.

### Glofitamab

The infusion of glofitamab is completed in observational units, infusion centers, or equivalent outpatient settings with immediate access to trained critical care personnel and facilities equipped to respond to and manage medical emergencies. Glofitamab is administered on a step-up dosing schedule starting on Cycle 1 Day 8 (2.5 mg), Cycle 1 Day 15 (10 mg), followed by 30 mg on Day 1 of Cycles 2-12, with each cycle being 21 days (i.e., every 3 weeks).

In the event of a late or prolonged infusion for glofitamab during dosing cycles 2-12 because of a late start in the day or slowed infusion rate due to an IRR or CRS, GemOx may be administered on the following day or as per local practice. There is a 90-minute observation period between the end of infusion of glofitamab and before GemOx infusion. At Cycle 3 and beyond, if the participant has not had CRS with prior cycles and has tolerated the preceding glofitamab infusion with no signs or symptoms of CRS, the window of observation may be shortened based on the discretion of the investigator.

On Cycle 1 Day 8, glofitamab is infused over 4 hours (±15 minutes), followed by 4 hours of monitoring prior to discharge. Participants need to remain in close proximity to the treating center for 24 hours and return the following day for follow-up. Participants and caregivers are educated regarding signs and symptoms of CRS. In the event of fever, or of feeling unwell, as described in the Patient Wallet Card, participants must immediately contact the treating team for medical direction such as emergency room or clinic visit. If the participant needs to go to a healthcare facility that is not the study site, the treating physician should contact the facility to coordinate care (contact details of study site is described in the Patient Wallet).

On Cycle 1 Day 15, glofitamab is infused over 4 hours (±15 minutes), followed by 4 hours of monitoring prior to discharge.

From Cycle 2 onwards, glofitamab is infused over 4 hours (±15 minutes), followed by 90 minutes of monitoring prior to discharge.

At Cycle 3 and beyond, if the participant has not had CRS with prior cycles and has tolerated the preceding glofitamab infusion with no signs or symptoms of CRS, the infusion time of glofitamab may be reduced to 2 hours (±15 minutes) and the window of observation may be shortened, based on the discretion of the investigator.

Participants with a Grade ≥2 CRS associated with the preceding dose of glofitamab may be monitored in a healthcare facility with appropriate medical support to manage CRS, at the discretion of the investigator for the next glofitamab dose administration.

In the event participants with Grade ≥2 CRS with the preceding glofitamab dose are not monitored in a healthcare facility, participants must remain within a reasonable distance from the study site or a facility with access to emergency medical care for at least 2 days following glofitamab administration. Participants should have a caregiver with them and should check in with a healthcare provider at the study site at least 1 day following glofitamab administration. During this time, participants must take their temperature regularly (morning, afternoon, and before they go to bed, at a minimum). In the event of fever, or of feeling unwell as described in the Patient Wallet Card, participants must immediately contact the treating team for medical direction, as described above.

### Tocilizumab

Tocilizumab is not administered to all participants but only to those participants who experience a CRS event for which tocilizumab is indicated. Prior to infusing glofitamab, the healthcare facility must have on-site access to tocilizumab, and ensure that a minimum of two doses of tocilizumab are available for the participant, if needed to treat CRS. Tocilizumab is supplied by the Sponsor as an IMP. Due to the need to manage CRS urgently and potential accessibility limitations at the study site, commercial tocilizumab can be obtained locally by the study site for emergency use purposes. It is prepared, handled, and managed according to standard institutional practices. Refer to the local prescribing information for further instructions regarding recommended storage conditions and packaging configuration.

For participants requiring treatment of CRS, tocilizumab is administered at a dose of 8 mg/kg IV (not exceeding 800 mg per infusion). If no clinical improvement in the signs and symptoms of CRS occurs after the first dose, a second dose may be administered at least 8 hours apart (maximum 2 doses per CRS event). Within each time period of 6 weeks of glofitamab treatment, the total number of tocilizumab doses does not exceed 3 doses.

If a participant experiences an infusion-associated adverse event with the 60-minute infusion despite premedication, all subsequent doses are delivered over 90 (±10) minutes. Similarly, if a participant experiences an infusion-associated adverse event with the 30-minute infusion despite premedication, all subsequent doses are delivered over 60 (±10) minutes.

### Gemcitabine

Gemcitabine is administered intravenously to participants at 1000 mg/m² on Cycle 1 Day 2. Starting at Cycle 2, gemcitabine may be given on Day 1 or 2 (per local practice) of each 21-day cycle (Cycles 2-8). Gemcitabine is administered before oxaliplatin on the same day.

### Oxaliplatin

Oxaliplatin is administered intravenously to participants at 100 mg/m² on Cycle 1 Day 2. Starting at Cycle 2, gemcitabine may be given on Day 1 or 2 (per local practice) of each 21-day cycle (Cycles 2-8). Oxaliplatin is administered after gemcitabine on the same day.

### Premedication

For the glofitamab step doses of 2.5 mg and 10 mg, participants receive 20 mg of dexamethasone orally one day prior to the glofitamab administration, 20 mg of dexamethasone IV 60 minutes prior to the glofitamab administration and 20 mg of dexamethasone orally one day after the glofitamab administration. For the target dose of 30 mg glofitamab, 20 mg dexamethasone is administered IV 60 minutes prior to glofitamab administration. Premedication with acetaminophen or paracetamol (500-1000 mg) and an antihistamine, such as diphenhydramine (50 mg), is administered approximately 30 minutes prior to the start of the infusion.

Dexamethasone premedication is optional at later cycles based on investigator's assessment for participants who have tolerated at least one 30 mg dose of glofitamab without experiencing CRS. However, if the participant experiences CRS, premedication with dexamethasone is required for subsequent glofitamab doses until no additional CRS events are observed.

TABLE 24
Premedications/prophylaxis before obinutuzumab and glofitamab infusions

| | Participants Requiring | Premedication/ | |
|---|---|---|---|
| Time point | Premedication | Prophylaxis | Administration |
| Obinutuzumab ^{a} | All participants | Hydration | Fluid intake of |
| Cycle 1, Day 1 | | | approximately 2-3 L/day |
| (and Cycle 1 | | | starting 24-48 hours |
| Day 2 if split | | | prior to the first dose of |
| dose) | | | study treatment |
| | | IV glucocorticoids ^{b} | At least 60 minutes prior |
| | | | to obinutuzumab |
| | | | infusion |
| | | Oral or IV analgesic or | At least 30 minutes prior |
| | | anti-pyretic medicine | to obinutuzumab |
| | | | infusion |
| | | Oral or IV | At least 30 minutes prior |
| | | antihistamine ^{c} | to obinutuzumab |
| | | | infusion |
| Obinutuzumab ^{a} | Participants at risk of TLS (e.g., | Allopurinol or suitable | 48-72 hours prior to |
| Cycle 1, Day 1 | because of bulky disease or | alternative, such as | study treatment |
| (or Cycle 1 Day | renal impairment creatinine | rasburicase, along with | |
| 2 if split dose) | clearance <70 mL/min) | adequate hydration | |
| (continued) | | | |
| Glofitamab ^{e, f} | All participants | Oral dexamethasone | One day prior (C1D7 |
| Cycle 1, Days | | | and C1D14) and one |
| 7-9 & Days 14-16 | | | day after (C1D9 and |
| | | | C1D16) to glofitamab |
| | | | infusion |
| | | IV dexamethasone ^{d, e} | At least 60 minutes prior |
| | | | to the glofitamab |
| | | | infusion (C1D8 and |
| | | | C1D15) |
| | | Oral or IV analgesic/ | At least 30 minutes prior |
| | | anti-pyretic medication | to glofitamab infusion |
| | | | (C1D8 and C1D15) |
| | | Oral or IV | At least 30 minutes prior |
| | | antihistamine ^{c} | to glofitamab infusion |
| | | | (C1D8 and C1D15) |
| | Participants at risk of TLS (e.g., | Allopurinol or suitable | 48□72 hours prior treatment |
| | because of bulky disease or | alternative, such as | |
| | renal impairment) (creatinine | rasburicase, along with | |
| | clearance <70 mL/min) | adequate hydration | |
| Glofitamab ^{e, f} | All participants | IV dexamethasone ^{d, e} | At least 60 minutes prior |
| Cycles 2-12, | | | to the first dose of study |
| Day 1 | | | treatment |
| | | Oral or IV analgesic/ | At least 30 minutes prior |
| | | anti-pyretic medication | to glofitamab infusion |
| | | Oral or IV | At least 30 minutes prior |
| | | antihistamine ^{c} | to glofitamab infusion |
| | Participants at risk of TLS (e.g., | Allopurinol or suitable | 48-72 hours prior to |
| | because of bulky disease or | alternative, such as | study treatment |
| | renal impairment) (creatinine | rasburicase, along with | |
| | clearance <70 mL/min) | adequate hydration | |

CRS = cytokine release syndrome; TLS = tumor lysis syndrome.
^{a} Closely monitor participants during the entire infusion. Infusion reactions within 24 hours of receiving obinutuzumab have occurred. Obinutuzumab is administered to well-hydrated participants.
^{b} IV glucocorticoid prior to obinutuzumab: dexamethasone 20 mg IV or methylprednisolone 80 mg IV.
^{c} For example, 50 mg diphenhydramine (unless contraindicated).
^{d} Administer dexamethasone 20 mg IV prior to glofitamab. If dexamethasone is not available, or the participant has an intolerance to dexamethasone, alternate corticosteroid (methylprednisolone 80 mg IV, or prednisone/prednisolone 100 mg) may be used for glofitamab premedication.
^{e} Optional at later cycles based on investigator's assessment for participants that have tolerated at least one 30 mg dose of glofitamab without experiencing CRS.
^{f} All glofitamab doses are administered to well-hydrated participants. Closely monitor participants during the entire infusion.

In case of infusion-associated adverse events in participants, the signs and symptoms should be fully resolved before the participant is discharged. Participants at risk of TLS should receive TLS prophylaxis (for details, refer to Table 24).

### Vital Signs

Temperature, pulse rate, respiratory rate, and blood pressure are assessed.

Blood pressure and pulse measurements are assessed while the participant is in a seated or semi-supine position with a completely automated device. Manual techniques are used only if an automated device is not available.

Blood pressure and pulse measurements are preceded by at least 5 minutes of rest for the participant in a quiet setting without distractions (e.g., television, cell phones).

Vital signs (to be taken before blood collection for laboratory tests) consist of one pulse and three blood pressure measurements (three consecutive blood pressure readings are recorded at intervals of at least 1 minute). The average of the three blood pressure readings is recorded on the eCRF.

Any abnormality identified at screening or prior to initiation of study treatment are recorded on the General Medical History and Baseline Conditions eCRF (unless considered related to a protocol-mandated intervention). After initiation of study treatment, any new or worsened clinically significant abnormalities are recorded as adverse events on the Adverse Event eCRF. Vital sign measurement may be performed by an MN professional.

### Vital Signs for Glofitamab Administration

On Cycle 1 Day 8, vital signs are collected as follows:
Pre-infusion, within 30 minutes prior to infusion.
Every 30 (±10) minutes during the infusion
At the end of infusion (EOI)+10 minutes.
Every 30 (±10) minutes during the 4 hour monitoring period

On Cycle 1 Day 15, vital signs are collected as follows:
Pre-infusion, within 30 minutes prior to infusion.
Every 30 (±10) minutes during the infusion.
At the EOI+10 minutes
Every 30 (±10) minutes during the 4 hour monitoring period. If the participant did not experience CRS during Cycle 1 Day 8, then vital signs are collected every 60 (±15) minutes during the 4 hour monitoring period.

For Cycles 2-12, if the participant tolerated Cycle 1 without the development of CRS, vital signs are collected as follows:
Pre-infusion, within 30 minutes prior to infusion.
Every 60 (±15) minutes during the infusion.
At the EOI+10 minutes
2-hours after the EOI+10 minutes

For Cycles 2-12, if the participant experienced CRS during Cycle 1, vital signs are collected as follows:
Pre-infusion, within 30 minutes prior to infusion.
Every 30 (±10) minutes during the infusion.
At the EOI+10 minutes
2-hours after the EOI+10 minutes

For participants with disease factors that may confer an increased risk of severe CRS during Cycle 1, vital signs are collected as follows:
Pre-infusion, within 30 minutes prior to infusion.
Every 15 (±5) minutes during the infusion.
At the EOI+5 minutes
Every 15 (±5) minutes post-infusion for 2 hours

### Estimation Methods for the Primary Endpoint

The primary objective of this study is to evaluate the frequency and severity of CRS, from the first study drug administration until the end-of-treatment, determined according to the ASTCT CRS grading criteria. The primary endpoint is defined as the number of participants
a) with a Grade 2 or higher CRS at any time.
b) With any Grade CRS, at any time.

These endpoints are summarized by descriptive statistics. A breakdown by grade is provided. The primary endpoint analyses are performed on the primary safety evaluable set. Sensitivity analyses are conducted on the modified safety evaluable set.

### Estimation Methods for the Secondary Endpoints

Secondary endpoints to evaluate objective of evaluating the frequency and severity of CRS:
Incidence of serious CRS events, with severity determined according to the ASTCT CRS grading criteria
   CRS frequency relative to the start of the infusion of each of the step-up doses of glofitamab.
   CRS management including outcome of participants who experience a CRS event

Secondary end points to evaluate objective of safety of Glofit-GemOx:
Incidence and severity of adverse events, with severity determined according to the NCI CTCAE v5.0
Tolerability, as assessed separately by:
   Dose interruptions
   Dose reductions
   Dose intensity

### Study Treatment Discontinuation Because of Adverse Events

Safety is assessed through summaries of exposure to study treatment, adverse events, changes in laboratory test results, and changes in vital signs and ECGs. Study treatment exposure (such as treatment duration, total dose received, and number of cycles and dose modifications) is summarized with descriptive statistics.

All verbatim adverse event terms are mapped to Medical Dictionary for Regulatory Activities (MedDRA) thesaurus terms. The adverse event severity grading scale for the NCI CTCAE v5.0 is used for assessing adverse event severity. All adverse events, serious adverse events, adverse events leading to death, adverse events of special interest, and adverse events leading to study treatment discontinuation that occur on or after the first dose of study treatment (i.e., treatment-emergent adverse events) str summarized by mapped term, appropriate thesaurus level, and severity grade. For events of varying severity, the highest grade is used in the summaries. Deaths and cause of death str summarized.

Relevant laboratory, vital sign (pulse rate, respiratory rate, blood pressure, pulse oximetry, and temperature), and ECG data are displayed by time, with grades identified where appropriate. Additionally, a shift table of selected laboratory tests is used to summarize the baseline and maximum postbaseline severity grade. Changes in vital signs and ECGs are summarized. As appropriate, listings, summary tables, and graphs are provided for safety and tolerability assessments.

All safety analyses are based on the primary safety evaluable population. Selected analyses are also conducted using the modified safety evaluable population.

The efficacy objective is to evaluate the efficacy of Glofit-GemOx in participants with R/R DLBCL on the basis of the following endpoints. The efficacy analyses are performed on the efficacy-evaluable set.

Investigator-assessed CRR, defined as the proportion of participants whose ORR is a CR based upon investigator assessment of PET-CT scans according to the Lugano Response Criteria (Cheson et al. 2014).

ORR, defined as the proportion of participants with a CR or partial response (PR) as determined by the investigator according to the Lugano Response Criteria (Cheson et al. 2014). DOR, defined as the time from the initial occurrence of a documented PR or CR until documented disease progression or death due to any cause, whichever occurs first. DOR is assessed by the investigator, using the Lugano Response Criteria (Cheson et al. 2014).

DOCR, defined as the time from the initial occurrence of a documented CR until documented disease progression or death due to any cause, whichever occurs first. DOCR is assessed by the investigator, using the Lugano Response Criteria (Cheson et al. 2014).

PFS, defined as the time from the first study treatment to the first occurrence of disease progression or death from any cause, whichever occurs first. PFS is assessed by the investigator, using the Lugano Response Criteria (Cheson et al. 2014).

Overall survival, defined as the time from the first study treatment to the date of death from any cause.

For CR rate and ORR, their 95% CIs are calculated using the Clopper-Pearson method. Responses after initiation of new anti-lymphoma therapy are not included in the analysis of CR rate and ORR.

The time-to-event endpoints, including DOR, DOCR, PFS, and OS, and time to deterioration in HRQoL are estimated with the use of Kaplan-Meier methodology. For OS, data from participants who did not have death documented are censored on the last date they were known to be alive.

### Example 8. FDA Information for Glofitamab (COLUMVI) for Treatment in Combination with GemOx

### Indication:

COLUMVI is a bispecific CD20-directed CD3 T-cell engager indicated in combination with gemcitabine and oxaliplatin for the treatment of adult patients with relapsed or refractory diffuse large B-cell lymphoma, not otherwise specified (DLBCL, NOS) who are not candidates for autologous stem cell transplant (ASCT).

### Dosing and Usage:

Pretreat with a single 1,000 mg dose of obinutuzumab intravenously 7 days before initiation of COLUMVI (Cycle 1 Day 1).

Administer only as an intravenous infusion through a dedicated infusion line that includes a sterile 0.2 micron in-line filter. Ensure adequate hydration before administering COLUMVI. Premedicate before each dose. Following pretreatment with obinutuzumab, administer COLUMVI according to the step-up dosing schedule in Table 25 with appropriate premedication, including dexamethasone, to reduce the incidence and severity of CRS.

Due to the risk of CRS, patients should be monitored for signs and symptoms of potential CRS for a combined total of 8 hours during and after completion of infusion of the first COLUMVI dose (2.5 mg on dosing cycle 1 (Cycle) 1 Day 8). Patients who experienced Grade ≥2 CRS with their previous infusion should be monitored after completion of the infusion.

TABLE 25
Dosing for COLUMVI

| Treatment | | Dose of COLUMVI | | Dose of | Dose of |
|---|---|---|---|---|---|
| cycle | Day | (duration of infusion) | | gemcitabine | oxaliplatin |
| Dosing cycle 1 | Day 1 | Obinutuzumab 1,000 mg^{a} | | | |
| | Day 2 | — | | 1,000 mg/m^{2 b} | 100 mg/m^{2 b} |
| | Day 8 | 2.5 mg | (4 hours)^{c} | — | — |
| | Day 15 | 10 mg | (4 hours)^{c} | | |
| Dosing cycle 2 | Day 1 | 30 mg | (4 hours)^{c, d} | 1,000 mg/m^{2 d} | 100 mg/m^{2 d} |
| Dosing cycle 3 | Day 1 | 30 mg | (2 hours) ^{d, e} | 1,000 mg/m^{2 d} | 100 mg/m^{2 d} |
| to 8 | | | | | |
| Dosing cycle 9 | Day 1 | 30 mg | (2 hours)^{e} | — | — |

to 12

^{a}Refer to “Pretreatment with obinutuzumab” described in this Example.
^{b} Cycle 1: Gemcitabine and oxaliplatin can be administered in any order.
^{c}For patients who experience CRS with their previous dose of COLUMVI, the time of infusion may be extended up to 8 hours.
^{d} Cycles 2-8: COLUMVI, gemcitabine and oxaliplatin can be administered in any order. Gemcitabine and oxaliplatin may be given on Day 1 or 2.
^{e}If the patient experienced CRS with the previous dose, the duration of infusion should be maintained at 4 hours.

Pretreat all patients with a single 1,000 mg dose of obinutuzumab administered as an intravenous infusion on Cycle 1 Day 1, 7 days prior to initiation of COLUMVI (see Table 25) to deplete the circulating and lymphoid tissue B cells. Obinutuzumab should be administered as an intravenous infusion at 50 mg/hour. The rate of infusion can be escalated in 50 mg/hour increments every 30 minutes to a maximum of 400 mg/hour.

COLUMVI dosing begins with a step-up dose schedule. Following completion of pretreatment with obinutuzumab on Cycle 1 Day 1, administer COLUMVI as an intravenous infusion according to the step-up dose schedule in Table 25. Administer premedications for each dose of COLUMVI as described in Table 27. Continue COLUMVI for a maximum of 12 dosing cycles (inclusive of Cycle 1 step-up dosing) or until disease progression or unacceptable toxicity, whichever occurs first. COLUMVI is given in combination with gemcitabine and oxaliplatin at Cycles 1-8 and as monotherapy at Cycles 9-12.

### Delayed or Missed Doses

If a dose of COLUMVI is delayed, restart therapy based on the recommendations made in Table 26, then resume the treatment schedule accordingly. When COLUMVI is given in combination with gemcitabine and oxaplatin, for repeat of the 2.5 mg dose patients should be monitored during and after completion of the COLUMVI infusion for a combined total of 8 hours. For the repeat of the 10 mg dose, patients should be monitored after completion of the COLUMVI infusion if Grade ≥2 CRS occurred during the most recent 2.5 mg dose.

TABLE 26
Recommendations for Restarting COLUMVI After Dose Delay

| Last Dose | Time Since Last | | |
|---|---|---|---|
| Administered | Dose Administered | | Action for Next Dose(s)^{a} |
| Obinutuzumab | ≤2 | weeks | Administer COLUMVI 2.5 mg (Cycle 1 Day 8)^{b}, then resume |
| pretreatment | | | the planned treatment schedule. |
| (Cycle 1 Day 1) | >2 | weeks | Repeat obinutuzumab 1,000 mg pretreatment (Cycle 1 |
| | | | Day 1). |
| | | | Then administer COLUMVI 2.5 mg (Cycle 1 Day 8)^{b} and |
| | | | resume the planned treatment schedule. |
| COLUMVI 2.5 mg | ≤2 | weeks | Administer COLUMVI 10 mg (Cycle 1 Day 15)^{c}, then resume |
| (Cycle 1 Day 8) | | | the planned treatment schedule. |
| | >2 to ≤4 | weeks | Repeat COLUMVI 2.5 mg (Cycle 1 Day 8)^{b}. |
| | | | Then administer COLUMVI 10 mg (Cycle 1 Day 15)^{c} and |
| | | | resume the planned treatment schedule. |
| | >4 | weeks | Repeat obinutuzumab 1,000 mg pretreatment (Cycle 1 |
| | | | Day 1) and COLUMVI 2.5 mg (Cycle 1 Day 8)^{b}. |
| | | | Then administer COLUMVI 10 mg (Cycle 1 Day 15)^{c} and |
| | | | resume the planned treatment schedule. |
| COLUMVI 10 mg | ≤2 | weeks | Administer COLUMVI 30 mg (Cycle 2 Day 1), then resume |
| (Cycle 1 Day 15) | | | the planned treatment schedule. |
| | >2 to ≤6 | weeks | Repeat COLUMVI 10 mg (Cycle 1 Day 15).^{c} |
| | | | Then administer COLUMVI 30 mg (Cycle 2 Day 1) and |
| | | | resume the planned treatment schedule. |
| | >6 | weeks | Repeat obinutuzumab 1,000 mg pretreatment (Cycle 1 |
| | | | Day 1), COLUMVI 2.5 mg (Cycle 1 Day 8)^{b}, and COLUMVI |
| | | | 10 mg (Cycle 1 Day 15)^{c}. |
| | | | Then administer COLUMVI 30 mg (Cycle 2 Day 1) and |
| | | | resume the planned treatment schedule. |
| COLUMVI 30 mg | ≤6 | weeks | Administer COLUMVI 30 mg, then resume the planned |
| (Cycle 2 onwards) | | | treatment schedule. |
| | >6 | weeks | Repeat the Cycle 1 regimen described in Table 25: |
| | | | obinutuzumab 1,000 mg pretreatment (Day 1), COLUMVI |
| | | | 2.5 mg (Day 8)^{b}, and COLUMVI 10 mg (Day 15)^{c}. |
| | | | Then administer COLUMVI 30 mg (Day 1 of next cycle) and |
| | | | resume the planned treatment schedule. |

^{a}Administer premedication as per Table 27 for all patients.
^{b}Patients receiving COLUMVI monotherapy should be hospitalized during and for 24 hours after completing infusion of the 2.5 mg dose. Patients receiving Columvi in combination should be monitored during and after completing infusion of the 2.5 mg dose, for a combined total of 8 hours.
^{c}Patients receiving COLUMVI monotherapy should be hospitalized during and for 24 hours after completing infusion of the 10 mg dose if CRS occurred during the most recent 2.5 mg dose. Patients receiving COLUMVI in combination should be monitored after completing infusion of the 10 mg dose if Grade ≥2 CRS occurred during the most recent 2.5 mg dose.

### Premedication

Administer the following premedications (Table 27) to reduce the risk of CRS and infusion-related reactions.

TABLE 27
Premedications to be Administered for COLUMVI Infusion

| Day of Treatment | Patients requiring | | |
|---|---|---|---|
| Cycle | premedication | Premedication | Administration |
| Cycle 1, Day 8 | All patients | Dexamethasone 20 mg | Completed at least 1 hour |
| and Day 15; | | intravenously* | prior to COLUMVI infusion. |
| Cycle 2; | | Acetaminophen 500 mg to | At least 30 minutes before |
| Cycle 3 | | 1,000 mg orally | COLUMVI infusion. |
| | | Antihistamine | Completed at least |
| | | (diphenhydramine 50 mg | 30 minutes before COLUMVI |
| | | orally or intravenously or | infusion. |
| | | equivalent) | |
| All subsequent | All patients | Acetaminophen 500 mg to | At least 30 minutes before |
| infusions | | 1,000 mg orally | COLUMVI infusion. |
| | | Antihistamine | Completed at least |
| | | (diphenhydramine 50 mg | 30 minutes before COLUMVI |
| | | orally or intravenously or | infusion. |
| | | equivalent) | |
| | Patients who | Dexamethasone 20 mg | Completed at least 1 hour |
| | experienced any | intravenously* | prior to COLUMVI infusion. |
| | grade CRS with the | | |
| | previous dose | | |

*If dexamethasone is not available, administer prednisone 100 mg, prednisolone 100 mg, or methylprednisolone 80 mg intravenously.

### Preparation and Administration

To dilute, withdraw the volume of 0.9% Sodium Chloride Injection or 0.45% Sodium Chloride Injection from the infusion bag according to Table 28 and discard. Withdraw the required volume of COLUMVI from vial(s) using a sterile needle and syringe and dilute into the infusion bag of 0.9% Sodium Chloride Injection or 0.45% Sodium Chloride Injection according to Table 28 to a final concentration of 0.1 mg/mL to 0.6 mg/mL. Discard any unused portion left in the vial.

TABLE 28
Dilution of COLUMVI for infusion

| | | | | Volume of 0.9% | | | |
|---|---|---|---|---|---|---|---|
| | | | | Sodium Chloride | | Volume of | |
| | | | | Injection or 0.45% | | COLUMVI to | |
| | | Size of | | Sodium Chloride | | be added in | |
| Dose of | | infusion | | Injection to be | | the infusion | |
| COLUMVI | | bag | | withdrawn and discarded | | bag | |
| 2.5 | mg | 50 | mL | 27.5 | mL | 2.5 | mL |
| 10 | mg | 50 | mL | 10 | mL | 10 | mL |
| | | 100 | mL | 10 | mL | 10 | mL |
| 30 | mg | 50 | mL | 30 | mL | 30 | mL |
| | | 100 | mL | 30 | mL | 30 | mL |

Gently invert infusion bag to mix the solution, in order to avoid excessive foaming. Do not shake. Immediately use diluted COLUMVI solution. If not used immediately, the diluted solution can be stored: either refrigerated at 2° C. to 8° C. (36° F. to 46° F.) for up to 64 hours or at room temperature up to 25° C. (77° F.) for up to 4 hours. Do not freeze the diluted infusion solution. Discard diluted infusion solution if storage time exceeds these limits. COLUMVI diluted with 0.9% Sodium Chloride Injection is compatible with intravenous infusion bags composed of polyvinyl chloride (PVC), polyethylene (PE), polypropylene (PP) or non-PVC polyolefin. When diluted with 0.45% Sodium Chloride Injection, COLUMVI is compatible with intravenous infusion bags composed of PVC.

Administer COLUMVI as an intravenous infusion only through a dedicated infusion line that includes a sterile 0.2-micron in-line filter. See Table 25 for duration of infusion. The maximum time for the administration of the diluted infusion solution may be extended up to 8 hours. Do not mix COLUMVI with other drugs. Injection solution (prior to dilution): 2.5 mg/2.5 mL (1 mg/mL) clear, colorless solution in a single-dose vial or 10 mg/10 mL (1 mg/mL) clear, colorless solution in a single-dose vial. Each mL of solution contains 1 mg glofitamab-gxbm, histidine (0.63 mg), histidine hydrochloride monohydrate (3.34 mg), methionine (1.49 mg), polysorbate 20 (0.5 mg), sucrose (82.15 mg), and Water for Injection, USP, at pH 5.5.

The pharmacokinetics (PK) of glofitamab-gxbm is not affected by co-administration with gemcitabine or oxaliplatin.

### Updated Results for STARGLO (Study GO41944; NCT04408638)

The efficacy of COLUMVI was evaluated in STARGLO (Study GO41944; NCT04408638), a randomized, open-label, multicenter clinical trial that included patients with relapsed or refractory DLBCL, NOS. The trial required an ECOG performance status of 0, 1, or 2, absolute neutrophil count ≥1,500/μL, platelet count ≥75,000/μL independent of transfusion, CrCL ≥30 mL/min, and hepatic transaminases ≤2.5×ULN. The trial excluded patients who received only one prior line of therapy who were candidates for stem cell transplant, patients with high-grade B-cell lymphoma, primary mediastinal B-cell lymphoma, history of transformation of indolent disease to DLBCL, patients with active or previous CNS lymphoma or CNS disease, patients with active or recent infections, or prior allogeneic HSCT.

Patients were randomized 2:1 to receive COLUMVI in combination with gemcitabine and oxaliplatin (COLUMVI+GemOx) or rituximab in combination with gemcitabine and oxaliplatin (R-GemOx) for 8 cycles, followed by 4 additional cycles of COLUMVI monotherapy for patients in the COLUMVI+GemOx arm. The cycle length was 21 days. COLUMVI was administered for up to 12 cycles in total unless patients experienced progressive disease or unacceptable toxicity. Randomization was stratified by number of previous lines of systemic therapy for DLBCL (1 vs. ≥2) and outcome of last systemic therapy (relapsed vs. refractory).

In the COLUMVI+GemOx arm, following pre-treatment with obinutuzumab on Cycle 1 Day 1, patients received 2.5 mg of COLUMVI on Cycle 1 Day 8, 10 mg of COLUMVI on Cycle 1 Day 15, and 30 mg of COLUMVI on Cycle 2 Day 1 as per the step-up dosing schedule. Patients continued to receive 30 mg of COLUMVI on Day 1 of Cycles 3 to 12. Gemcitabine 1000 mg/m² and oxaliplatin 100 mg/m² were administered intravenously on Day 2 of Cycle 1, and then on Day 1 of subsequent cycles, up to Cycle 8.

A total of 274 patients were randomized, with 183 in the COLUMVI+GemOx arm and 91 in the R-GemOx arm. The median age was 68 years (range: 20 to 88 years), 58% were male, 42% were white, 50% were Asian, 1.1% were Black or African American, and 6% were Hispanic or Latino. A majority of patients had ECOG performance status of 1 (47%) or 2 (10%). The majority of patients (63%) had received 1 prior line of systemic therapy and 37% of patients had received 2 or more prior lines of systemic therapy. All patients had received prior chemotherapy and most (99%) had received prior anti-CD20 monoclonal antibody therapy; 8% of patients had received CAR T-cell therapy, and 4.0% had received ASCT. Overall, 56% of patients had primary refractory disease and 61% of patients were refractory to the last therapy.

The primary endpoint was overall survival (OS). At the time of the prespecified primary analysis, a statistically significant improvement in OS was observed for patients randomized to the COLUMVI+GemOx arm compared to patients randomized to R-GemOx. Statistically significant improvements in PFS and CR rate, as assessed by IRC, were also observed with COLUMVI+GemOx over R-GemOx (Table 29).

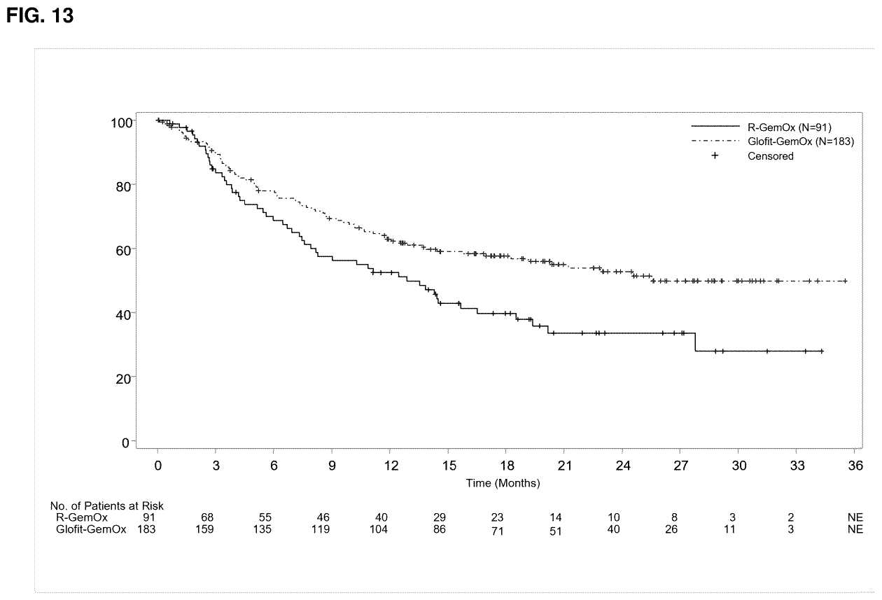

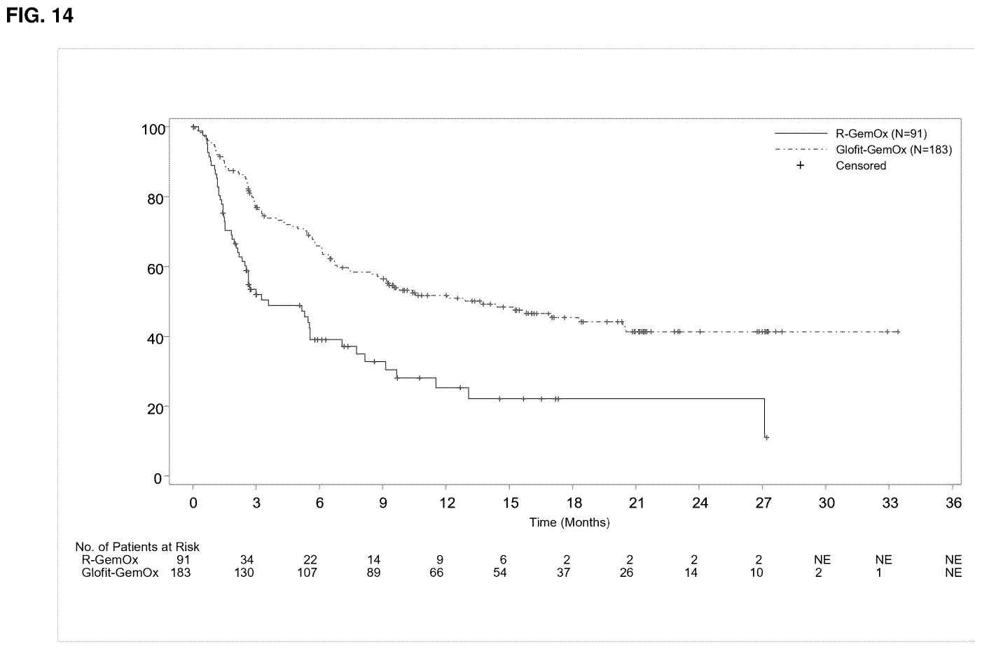

An updated analysis of OS, PFS, and CR conducted with an additional 10.5 months of follow-up continued to demonstrate benefit in the COLUMVI+GemOx arm (Table 29, FIG. 13 and FIG. 14). The 24-month OS rate was 53% [95% CI: 45, 61] in the COLUMVI+GemOx arm and 34% [95% CI: 22, 45] in the R-GemOx arm.

TABLE 29
Efficacy in Patients with Relapsed or Refractory DLBCL, NOS in STARGLO

| Primary Analysis | | Updated Analysis | |
|---|---|---|---|
| Median observation | | Median observation | |
| time = 11.3 months | | time = 20.7 months | |
| COLUMVI + GemOx | R-GemOx | COLUMVI + GemOx | R-GemOx |
| N = 183 | N = 91 | N = 183 | N = 91 |

Overall Survival

| | | | | | | | | |
|---|---|---|---|---|---|---|---|---|
| Number (%) of deaths | 61 | (33) | 40 | (44) | 80 | (44) | 52 | (57) |
| Median, months (95% CI) | NE | (13.8, NE) | 9.0 | (7.3, 14.4) | 25.5 | (18.3, NE) | 12.9 | (7.9, 18.5) |

| | | |
|---|---|---|
| HR (95% CI) | 0.59 (0.40, 0.89) | 0.62 (0.43, 0.88) |
| p-value | 0.011 | N/A |

Progression-free Survival- IRC-assessed

| | | | | | | | | |
|---|---|---|---|---|---|---|---|---|
| Number (%) of patients with | 68 | (37) | 44 | (48) | 90 | (49) | 54 | (59) |
| event | | | | | | | | |
| Median, months (95% CI) | 12.1 | (6.8, 18.3) | 3.3 | (2.5, 5.6) | 13.8 | (8.7, 20.5) | 3.6 | (2.5, 7.1) |

| | | |
|---|---|---|
| HR (95% CI) | 0.37 (0.25, 0.55) | 0.40 (0.28, 0.57) |
| p-value | <0.001 | N/A |

Complete Response Rate- IRC-assessed

| | | | | | | | | |
|---|---|---|---|---|---|---|---|---|
| Responders (%) | 92 | (50) | 20 | (22) | 107 | (59) | 23 | (25) |

| Difference in response | 28 (16, 40) | 33 (21, 45) |
|---|---|---|
| rate, % (95% CI) | | |
| p-value | <0.001 | N/A |

CI = confidence interval; HR = hazard ratio; NE = not estimable.

### Example 9. EMA Information for Glofitamab (Columvi) for Treatment in Combination with GemOx

### Indications

Columvi in combination with gemcitabine and oxaliplatin is indicated for the treatment of adult patients with relapsed or refractory diffuse large B-cell lymphoma not otherwise specified (DLBCL NOS) who are ineligible for autologous stem cell transplant (ASCT).

### Posology and Administration

At least 1 dose of tocilizumab for use in the event of CRS must be available prior to Columvi infusion at dosing cycles (Cycles) 1 and 2. Access to an additional dose of tocilizumab within 8 hours of use of the previous tocilizumab dose must be ensured.

All patients in study NP30179 and in study GO41944 (STARGLO) received a single 1000 mg dose of obinutuzumab as pre-treatment on Cycle 1 Day 1 (7 days prior to initiation of Columvi treatment) to lower the circulating and lymphoid B cells. Obinutuzumab was administered as an intravenous infusion at 50 mg/h. The rate of infusion was escalated in 50 mg/h increments every 30 minutes to a maximum of 400 mg/h.

Columvi should be administered to well hydrated patients. Recommended premedication for CRS is outlined in Table 30.

TABLE 30
Premedication before Columvi infusion

| Treatment | Patients requiring | | |
|---|---|---|---|
| cycle (Day) | premedication | Premedication | Administration |
| Cycle 1 | All patients | 20 mg intravenous | Completed at least 1 hour |
| (Day 8, Day 15); | | dexamethasone¹ | prior to Columvi infusion |
| Cycle 2 | | Oral analgesic/ | At least 30 minutes before |
| (Day 1); | | anti-pyretic² | Columvi infusion |
| Cycle 3 | | Anti-histamine³ | |
| (Day 1) | | | |
| All subsequent | All patients | Oral analgesic/ | At least 30 minutes before |
| infusions | | anti-pyretic² | |
| | | Anti-histamine³ | Columvi infusion |
| | Patients who experienced | 20 mg intravenous | Completed at least 1 hour |
| | CRS with the previous | dexamethasone^{1, 4} | prior to Columvi infusion |
| | dose | | |

¹If patient has an intolerance to dexamethasone or dexamethasone is unavailable, administer 100 mg prednisone/prednisolone or 80 mg methylprednisolone.
²For example, 1 000 mg paracetamol.
³For example, 50 mg diphenhydramine.
⁴To be administered in addition to the premedication required for all patients.

Columvi dosing begins with a step up dosing schedule (which is designed to decrease the risk of CRS), leading to the recommended dose of 30 mg. Columvi must be administered as an intravenous infusion according to the dose step-up schedule leading to the recommended dose of 30 mg (as shown in Table 31), after completion of pre-treatment with obinutuzumab on Cycle 1 Day 1. Columvi is given in combination with gemcitabine and oxaliplatin at Cycles 1-8 and as monotherapy at Cycles 9-12. Each cycle is 21 days.

TABLE 31
Columvi dose step-up schedule in combination with gemcitabine
and oxaliplatin for patients with relapsed or refractory DLBCL

| | | Dose of Columvi | | Dose of | | Dose of | |
|---|---|---|---|---|---|---|---|
| Treatment cycle, Day | | (duration of infusion) | | gemcitabine | | oxaliplatin | |
| Cycle 1 | Day 1 | Pre-treatment with obinutuzumab 1000 mg^{a} | | | | | |
| (Pre-treatment | Day 2 | — | | 1000 | mg/m^{2 b} | 100 | mg/m^{2 b} |
| and step-up dose) | Day 8 | 2.5 mg | (4 hours)^{c} | — | | — | |
| | Day 15 | 10 mg | (4 hours)^{c} | | | | |
| Cycle 2 | Day 1 | 30 mg | (4 hours)^{c, d} | 1000 | mg/m^{2 b, d} | 100 | mg/m^{2 b, d} |
| Cycle 3 to 8 | Day 1 | 30 mg | (2 hours)^{d, e} | 1000 | mg/m^{2 b, d} | 100 | mg/m^{2 b, d} |

| | | | | | |
|---|---|---|---|---|---|
| Cycle 9 to 12 | Day 1 | 30 mg | (2 hours)^{e} | — | — |

^{a}Refer to “Pre-treatment with obinutuzumab” described above.
^{b} Cycles 1-8: Administer gemcitabine before oxaliplatin.
^{c}For patients who experience CRS with their previous dose of Columvi, the time of infusion may be extended up to 8 hours (see section 4.4).
^{d}Cycles 2-8: Administer Columvi before gemcitabine and oxaliplatin. Gemcitabine and oxaliplatin may be given on Day 1 or 2.
^{e}Infusion time may be shortened to 2 hours at the discretion of the treating physician, if the previous infusion was well tolerated. If the patient experienced CRS with a previous dose, the duration of infusion should be maintained at 4 hours.

When Columvi is given in combination with gemcitabine and oxaliplatin, patients must be monitored for signs and symptoms of potential CRS during all Columvi infusions and for 4 hours after completion of the first Columvi dose (2.5 mg on Cycle 1 Day 8). Patients who experienced Grade ≥2 CRS with their previous infusion should be monitored after completion of the infusion. All patients must be monitored for signs and symptoms of CRS and immune effector cell-associated neurotoxicity syndrome (ICANS) following Columvi administration.

Treatment with Columvi in combination with gemcitabine and oxaliplatin is recommended for 8 cycles, followed by 4 cycles of Columvi monotherapy for a maximum of 12 cycles of Columvi in total or until disease progression or unmanageable toxicity, whichever occurs first. Each cycle is 21 days.

### For Delayed or Missed Doses:

During step up dosing (weekly dosing):

Following pre treatment with obinutuzumab, if the Columvi 2.5 mg dose is delayed by more than 1 week, then repeat pre treatment with obinutuzumab.

Following Columvi 2.5 mg dose or 10 mg dose, if there is a Columvi treatment free interval of 2 weeks to 6 weeks, then repeat the last tolerated Columvi dose and resume the planned step up dosing.

Following Columvi 2.5 mg dose or 10 mg dose, if there is a Columvi treatment free interval of more than 6 weeks, then repeat pre treatment with obinutuzumab and Columvi step up dosing (see Cycle 1 in Table 31).

After Cycle 2 (30 mg dose):

If there is a Columvi treatment free interval of more than 6 weeks between cycles, then repeat pre treatment with obinutuzumab and Columvi step up dosing (see Cycle 1 in Table 31), and then resume the planned treatment cycle (30 mg dose).

CRS should be identified based on the clinical presentation. Patients should be evaluated for other causes of fever, hypoxia, and hypotension, such as infections or sepsis. If CRS is suspected, it should be managed according to the CRS management recommendations based on American Society for Transplantation and Cellular Therapy (ASTCT) consensus grading.

No dose adjustment is required in patients 65 years of age and older.

No dose adjustment is required in patients with mild hepatic impairment (total bilirubin >upper limit of normal [ULN] to ≤1.5 □ ULN or aspartate transaminase [AST]>ULN). Columvi has not been studied in patients with moderate or severe hepatic impairment.

No dose adjustment is required in patients with mild or moderate renal impairment (CrCL 30 to <90 mL/min). Columvi has not been studied in patients with severe renal impairment.

The safety and efficacy of Columvi in children below 18 years of age have not been established. No data are available.

Columvi is for intravenous use only. Columvi must be diluted by a healthcare professional using aseptic technique, prior to intravenous administration. It must be administered as an intravenous infusion through a dedicated infusion line. Columvi must not be administered as an intravenous push or bolus.

Contraindication: Hypersensitivity to the active substance, to obinutuzumab, or to any of the excipients of columvi formulation (L histidine, L histidine hydrochloride monohydrate, L methionine, Sucrose, Polysorbate 20 (E432), Water for injections)

There are no data on the use of Columvi in pregnant women. No reproductive toxicity studies have been performed in animals.

Glofitamab is an immunoglobulin G (IgG). IgG is known to cross the placenta. Based on its mechanism of action, glofitamab is likely to cause foetal B-cell depletion when administered to a pregnant woman.

Columvi is not recommended during pregnancy and in women of childbearing potential not using contraception. Female patients receiving Columvi should be advised of the potential harm to the foetus. Female patients should be advised to contact the treating physician, should pregnancy occur.

It is not known whether glofitamab is excreted in human milk. No studies have been conducted to assess the impact of glofitamab on milk production or its presence in breast milk. Human IgG is known to be present in human milk. The potential for absorption of glofitamab and the potential for adverse reactions in the breast-feeding child is unknown. Women should be advised to discontinue breast feeding during treatment with Columvi and for 2 months after the final dose of Columvi.

No human data on fertility are available. No fertility assessments in animals have been performed to evaluate the effect of glofitamab on fertility.

Due to the potential for immune effector cell-associated neurotoxicity syndrome (ICANS), patients receiving Columvi are at risk of depressed level of consciousness. Patients should be instructed to avoid driving or operating machines for 48 hours after each of the first two doses during the step-up dosing and in the event of new onset of any symptoms of ICANS (confusion, disorientation, depressed level of consciousness) and/or CRS (pyrexia, tachycardia, hypotension, chills, hypoxia) until symptoms resolve.

### Undesirable Effects

The most common adverse reactions (≥20%) were thrombocytopenia, cytokine release syndrome, neutropenia, anaemia, nausea, peripheral neuropathy, diarrhoea, aspartate aminotransferase increased, alanine aminotransferase increased, rash, lymphopenia, pyrexia, and vomiting.

The most common serious adverse reactions reported in ≥2% of patients were cytokine release syndrome (20.3%), pyrexia (6.4%), pneumonia (5.8%), COVID-19 (5.8%), thrombocytopenia (4.7%), respiratory tract infection (3.5%), sepsis (2.3%), febrile neutropenia (2.3%), and diarrhoea (2.3%).

Permanent discontinuation of Columvi due to an adverse reaction occurred in 20.9% of patients. The most common adverse reactions leading to permanent discontinuation of Columvi were COVID-19 (11.6%), sepsis (1.2%), and pneumonitis (1.2%).

Adverse reactions occurring in relapsed or refractory DLBCL patients treated with Columvi in combination with gemcitabine and oxaliplatin (n=172) in study GO41944 (STARGLO) are listed in Table 32. Patients received a median of 11 cycles of Columvi treatment (range: 1 to 13 cycles).

The adverse reactions are listed by MedDRA system organ class and categories of frequency. The following categories of frequency have been used: very common (≥1/10), common (≥1/100 to <1/10), uncommon (≥1/1000 to <1/100), rare (≥1/10000 to <1/1000), very rare (<1/10000). Within each frequency grouping, adverse reactions are presented in the order of decreasing seriousness.

TABLE 32
Adverse reactions reported in patients with relapsed or refractory DLBCL
treated with Columvi in combination with gemcitabine and oxaliplatin

| System organ class | Adverse reaction | All grades | Grade 3-4 |
|---|---|---|---|
| Infections and infestations | COVID-19¹ | Very | Common* |
| | | common | |
| | Respiratory tract infections² | Very | Common* |
| | | common | |
| | Pneumonia³ | Very | Common* |
| | | common | |
| | Cytomegalovirus infections⁴ | Common | Uncommon |
| | Herpes viral infections⁵ | Common | Uncommon |
| | Urinary tract infection⁶ | Common | Common |
| | Sepsis⁷ | Common | Common* |
| | Candida infections⁸ | Common | Very rare** |
| | Pneumocystis jirovecii pneumonia | Uncommon | Uncommon |
| Neoplasms benign, malignant and | Tumour flare⁹ | Common | Very rare** |
| unspecified (incl cysts and | | | |
| polyps) | | | |
| Blood and lymphatic system | Thrombocytopenia | Very | Very |
| disorders | | common | common |
| | Neutropenia | Very | Very |
| | | common | common |
| | Anaemia | Very | Very |
| | | common | common |
| | Lymphopenia | Very | Very |
| | | common | common |
| | Febrile neutropenia | Common | Common |
| Immune system disorders | Cytokine release syndrome¹⁰ | Very | Common |
| | | common | |
| Metabolism and nutrition | Hypokalemia | Very | Common |
| disorders | | common | |
| | Hyponatraemia | Very | Uncommon |
| | | common | |
| | Hypomagnesaemia | Common | Very rare** |
| | Hypocalcaemia | Common | Uncommon |
| | Hypophosphataemia | Common | Common |
| | Tumour lysis syndrome | Common | Common |
| Nervous system disorders | Peripheral neuropathy¹¹ | Very | Common |
| | | common | |
| | Immune effector cell-associated | Common | Uncommon |
| | neurotoxicity syndrome¹² | | |
| | Headache | Common | Very rare** |
| | Tremor | Uncommon | Very rare** |
| Respiratory, thoracic and | Pneumonitis | Common | Very rare*_{,} ** |
| mediastinal disorders | | | |
| Gastrointestinal disorders | Nausea | Very | Uncommon |
| | | common | |
| | Diarrhoea | Very | Common |
| | | common | |
| | Vomiting | Very | Uncommon |
| | | common | |
| | Abdominal pain¹³ | Very | Common |
| | | common | |
| | Constipation | Very | Very rare** |
| | | common | |
| | Colitis¹⁴ | Common | Common |
| | Pancreatitis¹⁵ | Common | Common |
| Skin and subcutaneous tissue | Rash¹⁶ | Very | Uncommon |
| disorders | | common | |
| Musculoskeletal and connective | Musculoskeletal pain¹⁷ | Very | Common |
| tissue disorders | | common | |
| General disorders and | Pyrexia | Very | Uncommon |
| administration site conditions | | common | |
| Investigations | Aspartate aminotransferase increased | Very | Common |
| | | common | |
| | Alanine aminotransferase increased | Very | Common |
| | | common | |
| | Blood alkaline phosphatase increased | Very | Uncommon |
| | | common | |
| | Gamma-glutamyltransferase | Very | Common |
| | increased | common | |
| | Blood lactate dehydrogenase | Very | Very rare** |
| | increased | common | |
| | Blood bilirubin increased¹⁸ | Common | Very rare** |
| | Hepatic enzyme increased | Uncommon | Very rare** |

*Grade 5 reactions reported. See Description of selected adverse reactions.
**No Grade 3-4 events were reported.
¹Includes COVID-19, COVID-19 pneumonia, and SARS-CoV-2 test positive.
²Includes upper respiratory tract infection, lower respiratory tract infection, respiratory tract infection, and respiratory tract infection bacterial.
³Includes pneumonia, pneumonia bacterial, and pneumonia pneumococcal.
⁴New onset or reactivation. Includes cytomegalovirus infection, cytomegalovirus test positive, cytomegalovirus infection reactivation and cytomegalovirus viraemia.
⁵New onset or reactivation. Includes herpes zoster and herpes virus infection.
⁶Includes urinary tract infection and urosepsis.
⁷Includes sepsis, streptococcal sepsis, septic shock, and enterococcal sepsis.
⁸Includes oral candidiasis and candida infection.
⁹Includes tumour flare and tumour pain.
¹⁰Based on ASTCT consensus grading (Lee 2019).
¹¹Includes neuropathy peripheral, peripheral sensory neuropathy, dysaesthesia, paraesthesia, hypoaesthesia, peripheral motor neuropathy, and polyneuropathy.
¹²Includes confusional state, delirium, and ICANS.
¹³Includes abdominal pain, abdominal discomfort, abdominal pain upper, abdominal pain lower, and gastrointestinal pain.
¹⁴Includes colitis, colitis ischaemic, and enterocolitis.
¹⁵Includes pancreatitis and pancreatitis acute.
¹⁶Includes rash, rash pruritic, rash maculo-papular, erythema, pruritus, rash erythematous, urticaria, and erythema multiforme.
¹⁷Includes arthralgia, musculoskeletal pain, back pain, bone pain, myalgia, neck pain, pain in extremity, musculoskeletal chest pain, and non-cardiac chest pain.
¹⁸Includes blood bilirubin increased and hyperbilirubinaemia.

Any grade CRS (by ASTCT criteria) occurred in 44.2% of patients who received Columvi with gemcitabine and oxaliplatin, with Grade 1 CRS reported in 31.4% of patients, Grade 2 CRS in 10.5% of patients, and Grade 3 CRS in 2.3% of patients. CRS occurred more than once in 21.5% (37/172) of patients; 30/37 patients experienced multiple Grade 1 CRS events only. There were no Grade 4 or fatal cases of CRS. CRS resolved in all patients except one. One patient discontinued treatment due to CRS. In patients with CRS, the most common manifestations of CRS included pyrexia (98.7%), hypotension (22.4%), chills (17.1%) and hypoxia (14.5%). Grade 3 or higher events associated with CRS included hypotension (6.6%), hypoxia (5.3%), pyrexia (3.9%), chills (1.3%) and diarrhoea (1.3%). CRS of any grade occurred in 34.9% of patients following the first 2.5 mg dose of Columvi at Cycle 1 Day 8 with median time to onset (from start of infusion) of 12.6 hours (range: 4.4 to 54.7 hours) and median duration of 19.8 hours (range: 2.0 to 168.0 hours); in 14.4% of patients following the 10 mg dose at Cycle 1 Day 15 with median time to onset of 22.8 hours (range: 7.4 to 81.2 hours) and median duration of 10.6 hours (range: 1.0 to 248.5 hours); and in 9.3% of patients following the 30 mg dose at Cycle 2 with median time to onset of 23.5 hours (range: 14.7 to 33.4 hours) and median duration of 18.4 hours (range: 8.3 to 137.0 hours). CRS was reported in 6.7% of patients at Cycle 3 and in 11.0% of patients beyond Cycle 3. Grade ≥2 CRS occurred in 10.5% of patients following the first Columvi dose (2.5 mg) with median time to onset of 12.0 hours (range: 4.4 to 30.5 hours) and median duration of 42.3 hours (range: 3.5 to 143.7 hours). The majority (14/18) of patients who experienced Grade ≥2 CRS had onset of CRS within 8 hours of the start of the first Columvi dose (2.5 mg). Following Columvi 10 mg dose at Cycle 1 Day 15, the incidence of Grade ≥2 CRS decreased to 1.8% of patients with median time to onset of 22.3 hours (range: 7.4 to 22.8 hours) and median duration of 37.0 hours (range: 34.8 to 248.5 hours). There were no Grade ≥2 CRS events following Columvi 30 mg dose at Cycle 2 Day 1. Three patients (2.0%) had Grade ≥2 CRS beyond Cycle 2 (all Grade 2 events).

Of the 172 patients, 2 patients (1.2%) experienced elevated liver function tests (AST and ALT >3×ULN) reported concurrently with CRS.

Out of the 76 patients with any grade CRS, 28 patients (36.8%) were treated with tocilizumab, 39 patients (51.3%) were treated with corticosteroids, and 18 patients (23.7%) received both tocilizumab and corticosteroids. Among the 22 patients who experienced Grade ≥2 CRS after Columvi, 16 (72.7%) received tocilizumab, 15 (68.2%) received corticosteroids, and 12 (54.5%) received both tocilizumab and corticosteroids. Eleven patients (50.0%) received oxygen. All 4 patients (18.2%) with Grade 3 CRS received a single vasopressor. Hospitalisations due to patients experiencing CRS following Columvi administration occurred in 19.8% of patients and the reported median duration of hospitalisation was 5 days (range: 2 to 85 days).

ICANS, including Grade 3 and higher, was reported in clinical trials and with post-marketing experience. The most frequent clinical manifestations of ICANS were confusion, depressed level of consciousness, disorientation, seizure, aphasia, and dysgraphia. Based on the available data, the onset of neurologic toxicity was concurrent with CRS in the majority of cases. The observed time to onset of the majority of ICANS was 1-7 days with median of 2 days after the most recent dose. Only few events were reported to have occurred more than one month after the initiation of Columvi.

Serious infections were reported in 22.7% of patients who received Columvi with gemcitabine and oxaliplatin. The most frequent serious infections reported in ≥2% of patients were pneumonia (5.8%), COVID-19 (4.7%), and lower respiratory tract infection (2.9%). Infection related deaths were reported in 3.5% of patients (due to COVID-19, pneumonia, respiratory tract infection, and septic shock). One patient (0.6%) experienced a serious infection (pneumonia) concurrently with Grade 3 neutropenia.

Pneumonitis events (excluding pneumonia of infectious aetiology) were reported in 2 patients (1.2%) who received Columvi with gemcitabine and oxaliplatin, both of which were fatal events. The median time to onset of pneumonitis from the first Columvi dose was 168 days (range: 102 to 255 days).

Colitis events (excluding infectious aetiology) were reported in 4/172 patients (2.3%) who received Columvi with gemcitabine and oxaliplatin. Two patients (1.2%) had Grade 3 events. The median time to onset of colitis from the first Columvi dose was 154 days (range: 115 to 187 days).

Cytomegalovirus (CMV) events were reported in 10 patients (5.8%) who received Columvi with gemcitabine and oxaliplatin, with 1 patient (0.6%) experiencing Grade 3 CMV viraemia. Oral candidiasis was reported in 3 patients (1.7%) all of which were Grade 1-2 events. Pneumocystis jirovecii pneumonia (Grade 3) was reported in 1 patient (0.6%), the same patient with Grade 3 CMV viraemia. Borellia meningitis (Grade 2) was reported in 1 patient (0.6%).

### Product

Columvi (glofitamab) 2.5 mg concentrate for solution for infusion: each vial of 2.5 mL of concentrate contains 2.5 mg of glofitamab at a concentration of 1 mg/mL. 2.5 mL concentrate for solution for infusion in a 6 mL vial (colorless Type I glass) with stopper (butyl rubber). Pack size of 1 vial.

Columvi 10 mg concentrate for solution for infusion: each vial of 10 mL of concentrate contains 10 mg of glofitamab at a concentration of 1 mg/mL. 10 mL concentrate for solution for infusion in a 15 ml vial (colorless Type I glass) with stopper (butyl rubber). Pack size of 1 vial.

Colorless, clear solution with a pH of 5.5 and osmolality of 270-350 mOsm/kg.

Instructions for dilution: Columvi contains no preservative and is intended for single use only. Columvi must be diluted by a healthcare professional using aseptic technique, prior to intravenous administration. Visually inspect the Columvi vial for particulate matter or discoloration prior to administration. Columvi is a colorless, clear solution. Discard the vial if the solution is cloudy, discolored or contains visible particles. Withdraw the appropriate volume of sodium chloride 9 mg/ml (0.9%) solution for injection or sodium chloride 4.5 mg/mL (0.45%) solution for injection, as described in Table 33, from the infusion bag using a sterile needle and syringe and discard. Withdraw the required volume of Columvi concentrate for the intended dose from the vial using a sterile needle and syringe and dilute into the infusion bag (see Table 33). Discard any unused portion left in the vial. The final glofitamab concentration after dilution must be 0.1 mg/mL to 0.6 mg/mL. Gently invert the infusion bag to mix the solution in order to avoid excessive foaming. Do not shake. Inspect the infusion bag for particulates and discard if present. Prior to the start of the intravenous infusion, the content of the infusion bag should be at room temperature (25° C.).

TABLE 33
Dilution of Columvi for infusion

| | | | | Volume of sodium | | | |
|---|---|---|---|---|---|---|---|
| | | | | chloride 9 mg/mL (0.9%) | | | |
| | | | | or 4.5 mg/mL (0.45%) | | Volume of | |
| Dose of Columvi | | Size of | | solution for injection | | Columvi | |
| to be | | infusion | | to be withdrawn and | | concentrate | |
| administered | | bag | | discarded | | to be added | |
| 2.5 | mg | 50 | mL | 27.5 | mL | 2.5 | mL |
| | | 100 | mL | 77.5 | mL | 2.5 | mL |
| 10 | mg | 50 | mL | 10 | mL | 10 | mL |
| | | 100 | mL | 10 | mL | 10 | mL |
| 30 | mg | 50 | mL | 30 | mL | 30 | mL |
| | | 100 | mL | 30 | mL | 30 | mL |

Only sodium chloride 9 mg/ml (0.9%) or 4.5 mg/mL (0.45%) solution for injection should be used to dilute Columvi, since other solvents have not been tested. When diluted with sodium chloride 9 mg/ml (0.9%) solution for injection, Columvi is compatible with intravenous infusion bags composed of polyvinyl chloride (PVC), polyethylene (PE), polypropylene (PP) or non-PVC polyolefin. When diluted with sodium chloride 4.5 mg/mL (0.45%) solution for injection, Columvi is compatible with intravenous infusion bags composed of PVC. No incompatibilities have been observed with infusion sets with product-contacting surfaces of polyurethane (PUR), PVC or PE, and in-line filter membranes composed of polyethersulfone (PES) or polysulfone. The use of in-line filter membranes is optional.

### Example 10. Glofitamab Plus Gemcitabine and Oxaliplatin (Glofit-GemOx) in Patients with Relapsed/Refractory (R/R) Diffuse Large B-Cell Lymphoma (DLBCL): 2-Year Follow-Up of STARGLO

Presented here are updated efficacy and safety of Glofit-GemOx vs rituximab (R)-GemOx in patients with R/R DLBCL after ≥1 LOT from the Phase III STARGLO trial (NCT04408638), including landmark analyses of patients in complete remission (CR).

Methods: Patients were randomized 2:1 to Glofit-GemOx (8 cycles plus 4 cycles glofitamab monotherapy) or R-GemOx (8 cycles) and stratified by number of prior lines of therapy (LOT) (1 vs ≥2) and refractoriness to last therapy. After obinutuzumab pretreatment, glofitamab was given in dosing cycle (Cycle (C)) 1 as weekly step-up doses (2.5/10 mg) then 30 mg target dose every 21 days from C2 Day 1. Patients with only 1 prior LOT must have been autologous stem cell transplant (ASCT)-ineligible.

Primary endpoint was OS. Secondary endpoints included independent review committee (IRC)-assessed PFS and CR rate. A landmark analysis of patients in CR at end of treatment (EOT) was performed.

Results: Of 274 patients (Glofit-GemOx, n=183; R-GemOx, n=91), 172 (62.8%) had 1 prior LOT, 102 (37.2%) had ≥2 prior LOT, 153 (55.8%) were primary refractory, and 166 (60.6%) were refractory to last therapy. Baseline characteristics were unchanged and balanced across arms.

With 2 years follow-up (data cut off: Jun. 17, 2024; median follow-up: 24.7 months), Glofit-GemOx continued to confer superior OS benefits (median: not evaluable [NE] vs 13.5 months; hazard ratio [HR] 0.60, 95% confidence interval [CI]: 0.42-0.85), median IRC-assessed PFS (13.8 vs 3.6 months; HR 0.41, 95% CI: 0.29-0.58), and CR rate (58.5 vs 25.3%) vs R-GemOx. For Glofit-GemOx-treated patients in CR (n=107), median duration of CR was not reached (95% CI: 27.2-NE; median CR follow-up, 18.2 months [range: 15.2-19.3]). In patients with a CR at EOT (n=82), the OS and PFS rates 1 year after EOT were 89.3% and 82.4%, respectively.

The Glofit-GemOx safety profile was unchanged. Cytokine release syndrome (CRS) was the most common adverse event in glofitamab-exposed patients (Grade [Gr] 1, 32.0%; Gr 2, 10.5%; Gr 3, 2.3%). Events consistent with immune effector cell-associated neurotoxicity syndrome occurred in 4 patients (all concurrent with CRS; most Gr 1-2 [n=3]).

Summary/Conclusion: With 2 years follow-up, Glofit-GemOx sustained a clinically meaningful benefit in OS and PFS vs R-GemOx in ASCT-ineligible patients with R/R DLBCL, with most (82%) patients in CR at EOT still in remission. The safety profile was consistent with known risks of each drug. The updated analyses support the long-lasting remissions and maintained OS benefit in patients with R/R DLBCL treated with fixed duration Glofit-GemOx.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising:
(a) rituximab,
(b) gemcitabine, and
(c) oxaliplatin,
in the absence of glofitamab.

2. The method of claim 1, wherein;
(a) the PFS or the reference PFS is measured starting from the time from randomization to the time of a first occurrence of disease progression or death from any cause;
(b) the PFS or the reference PFS is the median PFS of the plurality of human patients receiving the corresponding treatment; and/or
(c) the improvement in PFS is statistically significant.

3-4. (canceled)

5. The method of claim 1, wherein:
(a) the improvement of the PFS is an increase in the median PFS of a plurality of human patients receiving the treatment compared to the median reference PFS of a plurality of human patients receiving the control treatment of between 1 and 16 months;
(b) administering such treatment to a plurality of human patients receiving the treatment results in a statistically significant improvement in the PFS as compared to a plurality of human patients receiving the control treatment with a hazard ratio of about 0.42 (95% confidence interval: 0.29, 0.61);
(c) administering such treatment to a plurality of human patients receiving the treatment results in a statistically significant improvement in the PFS as compared to a plurality of human patients receiving the control treatment with a hazard ratio of about 0.40 (95% confidence interval: 0.29, 0.61);
(d) administering such treatment to a plurality of human patients receiving the treatment results in a statistically significant improvement in the PFS as compared to a plurality of human patients receiving the control treatment with a hazard ratio of about 0.41 (95% confidence interval: 0.29, 0.58);
(e) administering such treatment to a plurality of human patients receiving the treatment results in an increase in the rate of PFS compared to the rate of reference PFS at 6 months of between 5% and 45%;
(f) administering such treatment to a plurality of human patients results in an increase in the rate of PFS compared to the rate of reference PFS at 12 months of between 5% and 45%; and/or
(g) administering such treatment to a plurality of human patients results in an improvement of the complete response rate (CR rate), objective response rate (ORR), duration of objective response, and/or duration of CR (DOCR) as compared to a plurality of human patients receiving the control treatment.

6. The method of claim 5, wherein:
(a) the improvement of the median PFS is an increase in the PFS compared to the reference PFS of about 9 months or about 10 months;
(b) the hazard ratio is a stratified hazard ratio;
(c) administering such treatment to a plurality of human patients results in an increase in the rate of PFS compared to the rate of reference PFS at 6 months of about 25%;
(d) administering such treatment to a plurality of human patients results in an increase in the rate of PFS compared to the rate of reference PFS at 12 months of about 25%;
(e) the CR rate is the proportion of patients whose best overall response is a CR on PET/computed tomography (CT);
(f) the improvement of the CR rate is between 15% and 50%;
(g) the ORR is the proportion of patients whose best overall response is a partial response (PR) or a CR; and/or
(h) the duration of objective response is measured as the time from the first occurrence of a documented objective response (CR or PR) to disease progression, or death from any cause, whichever occurs first.

7-18. (canceled)

19. The method of claim 6, wherein the improvement of the CR rate is an increase of about 30% or about 33%.

20-23. (canceled)

24. A method for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
wherein administering such treatment to a plurality of human patients results in an improvement in overall survival (OS) of the plurality of human patients as compared to a reference OS, wherein the reference OS is the OS of a plurality of human patients who have received a control treatment comprising:
(a) rituximab,
(b) gemcitabine, and
(c) oxaliplatin,
in the absence of glofitamab.

25. The method of claim 24, wherein:
(a) the OS or the reference OS is measured starting from the time from randomization to death from any cause;
(b) the OS or the reference OS is the median OS of the plurality of human patients receiving the corresponding treatment; and/or
(c) the improvement in OS is statistically significant.

26-27. (canceled)

28. The method of claim 25, wherein:
(a) the improvement of the median OS is an increase in the OS compared to the reference OS of between 1 and 30 months;
(b) administering such treatment to a plurality of human patients results in a statistically significant improvement in the OS as compared to the control treatment with a hazard ratio of about 0.62 (95% confidence interval: 0.43, 0.88);
(c) administering such treatment to a plurality of human patients results in a statistically significant improvement in the OS as compared to the control treatment with a hazard ratio of about 0.60 (95% confidence interval: 0.42, 0.85);
(d) administering such treatment to a plurality of human patients results in an increase in the rate of OS compared to the rate of reference OS at 12 months of between 5% and 30%;
(e) administering such treatment to a plurality of human patients results in an increase in the rate of OS compared to the rate of reference OS at 18 months of between 5% and 35%; and/or
(f) administering such treatment to a plurality of human patients results in an increase in the rate of OS compared to the rate of reference OS at 24 months of between 5% and 40%.

29. The method of claim 28, wherein:
(a) the improvement of the median OS is an increase in the OS compared to the reference OS of about 13 months;
(b) the hazard ratio is a stratified hazard ratio;
(c) administering such treatment to a plurality of human patients results in an increase in the rate of OS compared to the rate of reference OS at 12 months of about 10%;
(d) administering such treatment to a plurality of human patients results in an increase in the rate of OS compared to the rate of reference OS at 18 months of about 20%; and/or
(e) administering such treatment to a plurality of human patients results in an increase in the rate of OS compared to the rate of reference OS at 24 months of about 20%.

30-38. (canceled)

39. The method of claim 6, wherein the stratified hazard ratio is stratified by: (a) the number of previous lines of systemic therapy for DLBCL (1 vs. ≥2); and/or (b) the outcome of last systemic therapy (relapsed vs. refractory).

40. A method for treating relapsed or refractory diffuse large B-cell lymphoma (DLBCL) in a human patient in need thereof, comprising administering to the human patient an effective amount of:
(a) glofitamab,
(b) gemcitabine, and
(c) oxaliplatin,
wherein administering such treatment to the patient results in:
(a) a complete remission in the patient;
(b) a median duration of complete remission in the plurality of human patients of at least 27 months;
(c) a complete remission in the patient, and wherein a plurality of human patients having received such treatment and exhibiting complete remission after end of treatment exhibits a rate of overall survival at 12 months of about 89%; or
(d) a complete remission in the patient, and wherein a plurality of human patients having received such treatment and exhibiting complete remission after end of treatment exhibits a rate of progression-free survival at 12 months of about 82%.

41-43. (canceled)

44. The method of claim 1, wherein the method comprises a first and a second dosing cycle or 12 dosing cycles, wherein:
the first dosing cycle comprises a first dose (C1D1) of about 2.5 mg of the glofitamab and a second dose (C1D2) of about 10 mg the glofitamab, and
the second dosing cycle comprises a single dose (C2D1) of about 30 mg of the glofitamab.

45. The method of claim 44, wherein:
(a) the C1D1 and the C1D2 of the glofitamab are administered to the patient on Days 8 and 15, respectively, of the first dosing cycle;
(b) the C2D1 of the glofitamab is administered to the patient on Day 1 of the second dosing cycle;
(c) the first dosing cycle comprises a dose of 1000 mg of obinutuzumab;
(d) the first and the second dosing cycle comprise a dose of 1000 mg/m² of the gemcitabine and a dose of 100 mg/m² of the oxaliplatin; and/or
(e) the first and second dosing cycles are each 21-day dosing cycles or the dosing cycles are each 21-day dosing cycles.

46-47. (canceled)

48. The method of claim 45, wherein;
(a) 1000 mg of the obinutuzumab is administered about 7 days before the first glofitamab dose;
(b) the obinutuzumab is administered on Day 1 of the first dosing cycle;
(c) the gemcitabine and the oxaliplatin are administered on Day 2 of the first dosing cycle;
(d) gemcitabine and oxaliplatin are administered on Day 1 or 2 of the second dosing cycle; and/or
(e) gemcitabine is administered before oxaliplatin when administered on the same day.

49-57. (canceled)

58. The method of claim 45, wherein:
the obinutuzumab is administered at a dose of 1000 mg on Day 1 of dosing cycle 1;
the glofitamab is administered at a dose of 2.5 mg on Day 8 and 10 mg on Day 15 of dosing cycle 1; and at a dose of 30 mg on Day 1 of dosing cycles 2 to 12; and
the gemcitabine is administered at a dose of 1000 mg/m² and the oxaliplatin is administered at a dose of 100 mg/m² on Day 2 of dosing cycle 1 and on Day 1 or 2 of dosing cycles 2 to 8, wherein the gemcitabine is administered before the oxaliplatin when administered on the same day.

59. (canceled)

60. The method of claim 1, wherein;
(a) the DLBCL is a DLBCL not otherwise specified (DLBCL NOS);
(b) the patient is not a candidate for hematopoietic stem cell transplantation (HSCT); or
(c) the patient has a relapsed or refractory DLBCL NOS and is not a candidate for HSCT.

61-70. (canceled)

71. The method of claim 44, wherein the patient receives corticosteroid prophylaxis prior to and after administration of glofitamab.

72. The method of claim 71, wherein:
(a) the corticosteroid prophylaxis comprises prednisolone and methylprednisolone, and/or dexamethasone;
(b) the corticosteroid prophylaxis is administered one day prior to, on the same day as, and/or one day after administration of glofitamab;
(c) the corticosteroid prophylaxis is administered before the first dose (C1D1) of glofitamab;
(d) the corticosteroid prophylaxis is administered before the second dose (C1D2) of glofitamab;
(e) the corticosteroid prophylaxis is administered before the third dose (C2D1) of glofitamab;
(f) the corticosteroid prophylaxis is administered before any subsequent dose of glofitamab if the patient has experienced CRS with any previous 30 mg dose of glofitamab;
(g) the incidence of CRS in a plurality of patients is reduced compared to treatment wherein corticosteroid prophylaxis consists of one dose of a corticosteroid on the same day as glofitamab administration; and/or
(h) the patient does not need to be hospitalized after treatment with glofitamab.

73. (canceled)

74. The method of claim 72, wherein:
(a) the corticosteroid prophylaxis comprises 20 mg dexamethasone; and/or
(b) the corticosteroid prophylaxis is administered about 24 hours prior to the glofitamab administration, about 30-90 or about 60 minutes prior to the glofitamab administration, and about 24 hours after the glofitamab administration.

75-94. (canceled)

95. A method of reducing the incidence of CRS events or the likelihood of a CRS event in a CD20-positive B cell proliferative disorder patient population treated with glofitamab, comprising administering to the patients in the patient population glofitamab and dexamethasone in a dosing regimen comprising at least a first dosing cycle and a second dosing cycle, wherein:
(1) the first dosing cycle comprises a first dose (C1D1) of 2.5 mg of glofitamab and a second dose (C1D2) of 10 mg of glofitamab; and
(2) the second dosing cycle comprises a single dose (C2D1) of 30 mg of glofitamab, and
wherein dexamethasone is administered one day prior to administration of glofitamab, on the day of the administration of glofitamab, and one day after administration of glofitamab.

96. (canceled)

97. The method of claim 95, wherein;
(a) dexamethasone is administered at a dose of 20 mg;
(b) the dexamethasone is administered about 24 hours prior to administration of glofitamab, about 30-90 or about 60 minutes prior to the glofitamab administration, and about 24 hours after administration of glofitamab;
(c) the dexamethasone is administered for the first dose (C1D1) of glofitamab and the second dose (C1D2) of glofitamab;
(d) dexamethasone is administered orally;
(e) dexamethasone is administered for the third dose (C2D1) of glofitamab if the patient has experienced CRS with first and or second dose of glofitamab;
(f) dexamethasone is administered before any subsequent dose of glofitamab if the patient has experienced CRS with any previous 30 mg dose of glofitamab;
(g) the treatment does not cause Grade 3 or higher CRS (as defined by the American Society for Transplantation and Cellular Therapy, 2019; ASTCT);
(h) the rate of the cytokine release syndrome of any Grade (as defined by the ASTCT) is below 40%;
(i) the rate of the cytokine release syndrome of a Grade of 3 or greater (as defined by the ASTCT) is below 1%;
(i) the patient does not need to be hospitalized for the first two cycles of treatment with glofitamab; and/or
(k) the method further comprises administering gemcitabine and oxaliplatin.

98-112. (canceled)

113. The method of claim 24, wherein the method comprises a first and a second dosing cycle or 12 dosing cycles, wherein:
the first dosing cycle comprises a first dose (C1D1) of about 2.5 mg of the glofitamab and a second dose (C1D2) of about 10 mg the glofitamab, and
the second dosing cycle comprises a single dose (C2D1) of about 30 mg of the glofitamab.

114. The method of claim 113, wherein:
(a) the C1D1 and the C1D2 of the glofitamab are administered to the patient on Days 8 and 15, respectively, of the first dosing cycle;
(b) the C2D1 of the glofitamab is administered to the patient on Day 1 of the second dosing cycle;
(c) the first dosing cycle comprises a dose of 1000 mg of obinutuzumab;
(d) the first and the second dosing cycle comprise a dose of 1000 mg/m² of the gemcitabine and a dose of 100 mg/m² of the oxaliplatin; and/or
(e) the first and second dosing cycles are each 21-day dosing cycles or the dosing cycles are each 21-day dosing cycles.

115. The method of claim 114, wherein:
(a) 1000 mg of the obinutuzumab is administered about 7 days before the first glofitamab dose;
(b) the obinutuzumab is administered on Day 1 of the first dosing cycle;
(c) the gemcitabine and the oxaliplatin are administered on Day 2 of the first dosing cycle;
(d) gemcitabine and oxaliplatin are administered on Day 1 or 2 of the second dosing cycle; and/or
(e) gemcitabine is administered before oxaliplatin when administered on the same day.

116. The method of claim 114, wherein:
the obinutuzumab is administered at a dose of 1000 mg on Day 1 of dosing cycle 1;
the glofitamab is administered at a dose of 2.5 mg on Day 8 and 10 mg on Day 15 of dosing cycle 1; and at a dose of 30 mg on Day 1 of dosing cycles 2 to 12; and
the gemcitabine is administered at a dose of 1000 mg/m² and the oxaliplatin is administered at a dose of 100 mg/m² on Day 2 of dosing cycle 1 and on Day 1 or 2 of dosing cycles 2 to 8, wherein the gemcitabine is administered before the oxaliplatin when administered on the same day.

117. The method of claim 24, wherein:
(a) the DLBCL is a DLBCL NOS;
(b) the patient is not a candidate for HSCT; or
(c) the patient has a relapsed or refractory DLBCL NOS and is not a candidate for HSCT.

118. The method of claim 113, wherein the patient receives corticosteroid prophylaxis prior to and after administration of glofitamab.

119. The method of claim 118, wherein:
(a) the corticosteroid prophylaxis comprises prednisolone and methylprednisolone, and/or dexamethasone;
(b) the corticosteroid prophylaxis is administered one day prior to, on the same day as, and/or one day after administration of glofitamab;
(c) the corticosteroid prophylaxis is administered before the first dose (C1D1) of glofitamab;
(d) the corticosteroid prophylaxis is administered before the second dose (C1D2) of glofitamab;
(e) the corticosteroid prophylaxis is administered before the third dose (C2D1) of glofitamab;
(f) the corticosteroid prophylaxis is administered before any subsequent dose of glofitamab if the patient has experienced CRS with any previous 30 mg dose of glofitamab;
(g) the incidence of CRS in a plurality of patients is reduced compared to treatment wherein corticosteroid prophylaxis consists of one dose of a corticosteroid on the same day as glofitamab administration; and/or
(h) the patient does not need to be hospitalized after treatment with glofitamab.

120. The method of claim 119, wherein:
(a) the corticosteroid prophylaxis comprises 20 mg dexamethasone; and/or
(b) the corticosteroid prophylaxis is administered about 24 hours prior to the glofitamab administration, about 30-90 or about 60 minutes prior to the glofitamab administration, and about 24 hours after the glofitamab administration.


SEQUENCE LISTING (59 sequences)

SEQ ID NO: 1 - protein, 5 aa, synthetic construct
YSWIN

SEQ ID NO: 2 - protein, 17 aa, synthetic construct
RIFPGDGDTDYNGKFKG

SEQ ID NO: 3 - protein, 10 aa, synthetic construct
NVFDGYWLVY

SEQ ID NO: 4 - protein, 16 aa, synthetic construct
RSSKSLLHSNGITYLY

SEQ ID NO: 5 - protein, 7 aa, synthetic construct
QMSNLVS

SEQ ID NO: 6 - protein, 9 aa, synthetic construct
AQNLELPYT

SEQ ID NO: 7 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 9 - protein, 5 aa, synthetic construct
TYAMN

SEQ ID NO: 10 - protein, 19 aa, synthetic construct
RIRSKYNNYATYYADSVKG

SEQ ID NO: 11 - protein, 14 aa, synthetic construct
HGNFGNSYVSWFAY

SEQ ID NO: 12 - protein, 14 aa, synthetic construct
GSSTGAVTTSNYAN

SEQ ID NO: 13 - protein, 7 aa, synthetic construct
GTNKRAP

SEQ ID NO: 14 - protein, 9 aa, synthetic construct
ALWYSNLWV

SEQ ID NO: 15 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 16 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 17 - protein, 672 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 18 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
GGGGS

SEQ ID NO: 22 - protein, 5 aa, synthetic construct
SGGGG

SEQ ID NO: 23 - protein, 9 aa, synthetic construct
GGGGSGGGG

SEQ ID NO: 24 - protein, 20 aa, synthetic construct
GGGGSGGGGSGGGGSGGGGS

SEQ ID NO: 25 - protein, 12 aa, synthetic construct
GGGSGGGSGGGS

SEQ ID NO: 26 - protein, 16 aa, synthetic construct
GGGSGGGSGGGSGGGS

SEQ ID NO: 27 - protein, 20 aa, synthetic construct
GGGSGGGSGGGSGGGSGGGS

SEQ ID NO: 28 - protein, 24 aa, synthetic construct
GGGSGGGSGGGSGGGSGGGSGGGS

SEQ ID NO: 29 - protein, 10 aa, synthetic construct
GGGGSGGGGS

SEQ ID NO: 30 - protein, 15 aa, synthetic construct
GGGGSGGGGSGGGGS

SEQ ID NO: 31 - protein, 20 aa, synthetic construct
GGGGSGGGGSGGGGSGGGGS

SEQ ID NO: 32 - protein, 25 aa, synthetic construct
GGGGSGGGGSGGGGSGGGGSGGGGS

SEQ ID NO: 33 - protein, 13 aa, synthetic construct
GGGSGGGSGGGSG

SEQ ID NO: 34 - protein, 17 aa, synthetic construct
GGGSGGGSGGGSGGGSG

SEQ ID NO: 35 - protein, 21 aa, synthetic construct
GGGSGGGSGGGSGGGSGGGSG

SEQ ID NO: 36 - protein, 25 aa, synthetic construct
GGGSGGGSGGGSGGGSGGGSGGGSG

SEQ ID NO: 37 - protein, 11 aa, synthetic construct
GGGGSGGGGSG

SEQ ID NO: 38 - protein, 16 aa, synthetic construct
GGGGSGGGGSGGGGSG

SEQ ID NO: 39 - protein, 21 aa, synthetic construct
GGGGSGGGGSGGGGSGGGGSG

SEQ ID NO: 40 - protein, 26 aa, synthetic construct
GGGGSGGGGSGGGGSGGGGSGGGGSG

SEQ ID NO: 41 - protein, 14 aa, synthetic construct
GGGSGGGSGGGSGG

SEQ ID NO: 42 - protein, 18 aa, synthetic construct
GGGSGGGSGGGSGGGSGG

SEQ ID NO: 43 - protein, 22 aa, synthetic construct
GGGSGGGSGGGSGGGSGGGSGG

SEQ ID NO: 44 - protein, 26 aa, synthetic construct
GGGSGGGSGGGSGGGSGGGSGGGSGG

SEQ ID NO: 45 - protein, 12 aa, synthetic construct
GGGGSGGGGSGG

SEQ ID NO: 46 - protein, 17 aa, synthetic construct
GGGGSGGGGSGGGGSGG

SEQ ID NO: 47 - protein, 22 aa, synthetic construct
GGGGSGGGGSGGGGSGGGGSGG

SEQ ID NO: 48 - protein, 27 aa, synthetic construct
GGGGSGGGGSGGGGSGGGGSGGGGSGG

SEQ ID NO: 49 - protein, 15 aa, synthetic construct
GGGSGGGSGGGSGGG

SEQ ID NO: 50 - protein, 19 aa, synthetic construct
GGGSGGGSGGGSGGGSGGG

SEQ ID NO: 51 - protein, 23 aa, synthetic construct
GGGSGGGSGGGSGGGSGGGSGGG

SEQ ID NO: 52 - protein, 27 aa, synthetic construct
GGGSGGGSGGGSGGGSGGGSGGGSGGG

SEQ ID NO: 53 - protein, 13 aa, synthetic construct
GGGGSGGGGSGGG

SEQ ID NO: 54 - protein, 18 aa, synthetic construct
GGGGSGGGGSGGGGSGGG

SEQ ID NO: 55 - protein, 23 aa, synthetic construct
GGGGSGGGGSGGGGSGGGGSGGG

SEQ ID NO: 56 - protein, 28 aa, synthetic construct
GGGGSGGGGSGGGGSGGGGSGGGGSGGG

SEQ ID NO: 57 - protein, 11 aa, synthetic construct
DGGGGSGGGGS

SEQ ID NO: 58 - protein, 674 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 449 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK